SEQUENCE LISTING (67 sequences)

SEQ ID NO: 1 - skipped

SEQ ID NO: 2 - RNA, 57 nt, Artificial Sequence
  Synthetic
gggaaauaagagagaaaagaagaguaagaagaaauauaagaccccggcgccgccacc

SEQ ID NO: 3 - skipped

SEQ ID NO: 4 - RNA, 119 nt, Artificial Sequence
  Synthetic
ugauaauaggcuggagccucgguggccuagcuucuugccccuugggccuccccccagccccuccuccccuuccugcacccguacccccguggucuuugaauaaagucugagugggcggc

SEQ ID NO: 5-17 - skipped (13 entries)

SEQ ID NO: 18 - RNA, 1748 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 19 - RNA, 1572 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 20 - protein, 524 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKSGGGSILAIYSTVASSLVLLVSLGAISF

SEQ ID NO: 21 - RNA, 1748 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccuacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 22 - RNA, 1572 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccuacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 23 - protein, 524 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKSGGGSILAIYSTVASSLVLLVSLGAISF

SEQ ID NO: 24 - RNA, 1739 nt, Artificial Sequence
  Synthetic
gggaaauaagagagaaaagaagaguaagaagaaauauaagaccccggcgccgccaccauguucguguuccuggugcugcugccccuggugagcagccagugcgugaacuuuaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgccaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcggggccugccccagggcuucucagcccuggagccccugguggaccugcccaucggcaucaacaucacccgguuccagacccugcacaucagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccuacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 25 - RNA, 1563 nt, Artificial Sequence
  Synthetic
auguucguguuccuggugcugcugccccuggugagcagccagugcgugaacuuuaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgccaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcggggccugccccagggcuucucagcccuggagccccugguggaccugcccaucggcaucaacaucacccgguuccagacccugcacaucagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccuacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 26 - protein, 521 aa, Artificial Sequence
  Synthetic
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKSGGGSILAIYSTVASSLVLLVSLGAISF

SEQ ID NO: 27 - RNA, 1739 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccaucagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccuacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 28 - RNA, 1563 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccaucagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccuacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 29 - protein, 521 aa, Artificial Sequence
  Synthetic
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKSGGGSILAIYSTVASSLVLLVSLGAISF

SEQ ID NO: 30 - RNA, 1730 nt, Artificial Sequence
  Synthetic
gggaaauaagagagaaaagaagaguaagaagaaauauaagaccccggcgccgccaccauguucguguuccuggugcugcugccccuggugagcagccagugcgugaacuuuaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccaucagcggcaccaacggcaccaagcgguucgccaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcggggccugccccagggcuucucagcccuggagccccugguggaccugcccaucggcaucaacaucacccgguuccagacccugcacaucagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccuacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 31 - RNA, 1554 nt, Artificial Sequence
  Synthetic
auguucguguuccuggugcugcugccccuggugagcagccagugcgugaacuuuaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccaucagcggcaccaacggcaccaagcgguucgccaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcggggccugccccagggcuucucagcccuggagccccugguggaccugcccaucggcaucaacaucacccgguuccagacccugcacaucagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcgugaagggcuucaacugcuacuucccucugcagagcuacggcuuccagcccaccuacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 32 - protein, 518 aa, Artificial Sequence
  Synthetic
MFVFLVLLPLVSSQCVNFTTRTQLPPAYTNSFTRGVYYPDKVFRSSVLHSTQDLFLPFFSNVTWFHAISGTNGTKRFANPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKSGGGSILAIYSTVASSLVLLVSLGAISF

SEQ ID NO: 33 - RNA, 47 nt, Artificial Sequence
  Synthetic
gggaaauaagagagaaaagaagaguaagaagaaauauaagagccacc

SEQ ID NO: 34 - RNA, 119 nt, Artificial Sequence
  Synthetic
ugauaauaggcuggagccucgguggccaugcuucuugccccuugggccuccccccagccccuccuccccuuccugcacccguacccccguggucuuugaauaaagucugagugggcggc

SEQ ID NO: 35 - protein, 10 aa, Artificial Sequence
  Synthetic
TQTNSPRRAR

SEQ ID NO: 36 - protein, 1273 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 37 - RNA, 9 nt, Artificial Sequence
  Synthetic
ccrccaugg

SEQ ID NO: 38 - RNA, 11 nt, Artificial Sequence
  Synthetic
gggauccuacc

SEQ ID NO: 39 - RNA, 3995 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacaagguggaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacaccugauaauaggcuggagccucgguggccuagcuucuugccccuugggccuccccccagccccuccuccccuuccugcacccguacccccguggucuuugaauaaagucugagugggcggc

SEQ ID NO: 40 - RNA, 3819 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaccgagaagagcaacaucauccggggcuggaucuucggcaccacccuggacagcaagacccagagccugcugaucgugaauaacgccaccaacguggugaucaaggugugcgaguuccaguucugcaacgaccccuuccugggcguguacuaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccguggagaagggcaucuaccagaccagcaacuuccgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuaccuguaccggcuguuccggaagagcaaccugaagcccuucgagcgggacaucagcaccgagaucuaccaagccggcuccaccccuugcaacggcguggagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcuucggcggcgugagcgugaucaccccaggcaccaacaccagcaaccagguggccgugcuguaccaggacgugaacugcaccgaggugcccguggccauccacgccgaccagcugacacccaccuggcgggucuacagcaccggcagcaacguguuccagacccgggccgguugccugaucggcgccgagcacgugaacaacagcuacgagugcgacauccccaucggcgccggcaucugugccagcuaccagacccagaccaauucaccccggagggcaaggagcguggccagccagagcaucaucgccuacaccaugagccugggcgccgagaacagcguggccuacagcaacaacagcaucgccauccccaccaacuucaccaucagcgugaccaccgagauucugcccgugagcaugaccaagaccagcguggacugcaccauguacaucugcggcgacagcaccgagugcagcaaccugcugcugcaguacggcagcuucugcacccagcugaaccgggcccugaccggcaucgccguggagcaggacaagaacacccaggagguguucgcccaggugaagcagaucuacaagaccccucccaucaaggacuucggcggcuucaacuucagccagauccugcccgaccccagcaagcccagcaagcggagcuucaucgaggaccugcuguucaacaaggugacccuagccgacgccggcuucaucaagcaguacggcgacugccucggcgacauagccgcccgggaccugaucugcgcccagaaguucaacggccugaccgugcugccuccccugcugaccgacgagaugaucgcccaguacaccagcgcccuguuagccggaaccaucaccagcggcuggacuuucggcgcuggagccgcucugcagauccccuucgccaugcagauggccuaccgguucaacggcaucggcgugacccagaacgugcuguacgagaaccagaagcugaucgccaaccaguucaacagcgccaucggcaagauccaggacagccugagcagcaccgcuagcgcccugggcaagcugcaggacguggugaaccagaacgcccaggcccugaacacccuggugaagcagcugagcagcaacuucggcgccaucagcagcgugcugaacgacauccugagccggcuggacaagguggaggccgaggugcagaucgaccggcugaucacuggccggcugcagagccugcagaccuacgugacccagcagcugauccgggccgccgagauucgggccagcgccaaccuggccgccaccaagaugagcgagugcgugcugggccagagcaagcggguggacuucugcggcaagggcuaccaccugaugagcuuuccccagagcgcaccccacggagugguguuccugcacgugaccuacgugcccgcccaggagaagaacuucaccaccgccccagccaucugccacgacggcaaggcccacuuuccccgggagggcguguucgugagcaacggcacccacugguucgugacccagcggaacuucuacgagccccagaucaucaccaccgacaacaccuucgugagcggcaacugcgacguggugaucggcaucgugaacaacaccguguacgauccccugcagcccgagcuggacagcuucaaggaggagcuggacaaguacuucaagaaucacaccagccccgacguggaccugggcgacaucagcggcaucaacgccagcguggugaacauccagaaggagaucgaucggcugaacgagguggccaagaaccugaacgagagccugaucgaccugcaggagcugggcaaguacgagcaguacaucaaguggcccugguacaucuggcugggcuucaucgccggccugaucgccaucgugauggugaccaucaugcugugcugcaugaccagcugcugcagcugccugaagggcuguugcagcugcggcagcugcugcaaguucgacgaggacgacagcgagcccgugcugaagggcgugaagcugcacuacacc

SEQ ID NO: 41 - RNA, 1748 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaauaacgccaccaacguggugaucaaggugugcgaguuccaguucugcaacgaccccuuccuggacguguacuaccacaagaacaacaagagcuggaugaagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuacagauaccggcuguuccggaagagcaaccugaagcccuucgagcgggacaucagcaccgagaucuaccaagccggcuccaccccuugcaacggcgugcagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 42 - RNA, 1572 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaauaacgccaccaacguggugaucaaggugugcgaguuccaguucugcaacgaccccuuccuggacguguacuaccacaagaacaacaagagcuggaugaagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuacagauaccggcuguuccggaagagcaaccugaagcccuucgagcgggacaucagcaccgagaucuaccaagccggcuccaccccuugcaacggcgugcagggcuucaacugcuacuucccucugcagagcuacggcuuccagcccaccaacggcgugggcuaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 43 - protein, 524 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDGGGSGGGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKSGGGSILAIYSTVASSLVLLVSLGAISF

SEQ ID NO: 44 - RNA, 2024 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaauaacgccaccaacguggugaucaaggugugcgaguuccaguucugcaacgaccccuuccuggacguguacuaccacaagaacaacaagagcuggaugaagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccggaggcggaucgggaggcggacagcgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuacagauaccggcuguuccggaagagcaaccugaagcccuucgagcgggacaucagcaccgagaucuaccaagccggcuccaccccuugcaacggcgugcagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcucuggcggaggcagcauccuggccaucuacagcaccguggccagcagccuggugcuccuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 45 - RNA, 1848 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgccauccacgugagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaauaacgccaccaacguggugaucaaggugugcgaguuccaguucugcaacgaccccuuccuggacguguacuaccacaagaacaacaagagcuggaugaagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaaccuggugcgggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacugcgcccuggacccucugagcgagaccaagugcacccugaagagcuucaccggaggcggaucgggaggcggacagcgggugcagcccaccgagagcaucgugcgguuccccaacaucaccaaccugugccccuucggcgagguguucaacgccacccgguucgccagcguguacgccuggaaccggaagcggaucagcaacugcguggccgacuacagcgugcuguacaacagcgccagcuucagcaccuucaagugcuacggcgugagccccaccaagcugaacgaccugugcuucaccaacguguacgccgacagcuucgugauccguggcgacgaggugcggcagaucgcacccggccagacaggcaagaucgccgacuacaacuacaagcugcccgacgacuucaccggcugcgugaucgccuggaacagcaacaaccucgacagcaaggugggcggcaacuacaacuacagauaccggcuguuccggaagagcaaccugaagcccuucgagcgggacaucagcaccgagaucuaccaagccggcuccaccccuugcaacggcgugcagggcuucaacugcuacuucccucugcagagcuacggcuuccagcccaccaacggcgugggcuaccagcccuaccgggugguggugcugagcuucgagcugcugcacgccccagccaccguguguggccccaagaagagcaccaaccuggugaagaacaagugcgugaacuucaacuucaacggccuuaccggcaccggcgugcugaccgagagcaacaagaaauuccugcccuuucagcaguucggccgggacaucgccgacaccaccgacgcugugcgggauccccagacccuggagauccuggacaucaccccuugcagcucuggcggaggcagcauccuggccaucuacagcaccguggccagcagccuggugcuccuggugagccugggcgccaucagcuuc

SEQ ID NO: 46 - protein, 616 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTGGGSGGGQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSSGGGSILAIYSTVASSLVLLVSLGAISF

SEQ ID NO: 47-54 - skipped (8 entries)

SEQ ID NO: 55 - RNA, 1739 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgugaucagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaauaacgccaccaacguggugaucaaggugugcgaguuccaguucugcaacgaccccuuccuggaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaucgugcgggagcccgaggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucgacgagguguucaacgccacccgguucgccagcguguacgccuggaaccggaagcggaucagcaacugcguggccgacuacagcgugcuguacaaccuggcccccuucuucaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaagcucgacagcaaggugagcggcaacuacaacuaccuguaccggcuguuccggaagagcaaccugaagcccuucgagcgggacaucagcaccgagaucuaccaagccggcaacaagccuugcaacggcguggccggcuucaacugcuacuucccucugcggagcuacagcuuccggcccaccuacggcgugggccaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 56 - RNA, 1563 nt, Artificial Sequence
  Synthetic
auguucguguuccuggugcugcugccccuggugagcagccagugcgugaaccugaccacccggacccagcugccaccagccuacaccaacagcuucacccggggcgucuacuaccccgacaagguguuccggagcagcguccugcacagcacccaggaccuguuccugcccuucuucagcaacgugaccugguuccacgugaucagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaauaacgccaccaacguggugaucaaggugugcgaguuccaguucugcaacgaccccuuccuggaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaucgugcgggagcccgaggaucugccccagggcuucucagcccuggagccccugguggaccugcccaucggcaucaacaucacccgguuccagacccugcuggcccugcaccggagcuaccugaccccaggcgacagcagcagcggguggacagcaggcgcggcugcuuacuacgugggcuaccugcagccccggaccuuccugcugaaguacaacgagaacggcaccaucaccgacgccguggacggaggcggaucgggaggcggacccaacaucaccaaccugugccccuucgacgagguguucaacgccacccgguucgccagcguguacgccuggaaccggaagcggaucagcaacugcguggccgacuacagcgugcuguacaaccuggcccccuucuucaccuucaagugcuacggcgugagccccaccaagcugaacgaccugugcuucaccaacguguacgccgacagcuucgugauccguggcgacgaggugcggcagaucgcacccggccagacaggcaacaucgccgacuacaacuacaagcugcccgacgacuucaccggcugcgugaucgccuggaacagcaacaagcucgacagcaaggugagcggcaacuacaacuaccuguaccggcuguuccggaagagcaaccugaagcccuucgagcgggacaucagcaccgagaucuaccaagccggcaacaagccuugcaacggcguggccggcuucaacugcuacuucccucugcggagcuacagcuuccggcccaccuacggcgugggccaccagcccuaccgggugguggugcugagcuucgagcugcugcacgccccagccaccguguguggccccaagucuggcggaggcagcauccuggccaucuacagcaccguggccagcagccuggugcugcuggugagccugggcgccaucagcuuc

SEQ ID NO: 57 - protein, 521 aa, Artificial Sequence
  Synthetic
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDGGGSGGGPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKSGGGSILAIYSTVASSLVLLVSLGAISF

SEQ ID NO: 58 - RNA, 1739 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccuccugccuacaccaacagcuucacccggggcguguacuaccccgacaagguguuccggagcagcgugcugcacagcacccaggaccuguuccugcccuucuucagcaacgugaccugguuccacgugaucagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaacaacgccaccaacguggugaucaaggugugcgaguuccaguucugcaacgaccccuuccuggaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccuaucaucgugcgggagcccgaggaccugccccagggcuucagcgcccuggagccccugguggaccugcccaucggcaucaacaucacccgguuccagacccugcuggcccugcaccggagcuaccugaccccuggcgacagcagcagcggcuggaccgccggcgccgccgccuacuacgugggcuaccugcagccccggaccuuccugcugaaguacaacgagaacggcaccaucaccgacgccguggacggcggcggcagcggcggcggccccaacaucaccaaccugugccccuucgacgagguguucaacgccacccgguucgccagcguguacgccuggaaccggaagcggaucagcaacugcguggccgacuacagcgugcuguacaaccuggcccccuucuucaccuucaagugcuacggcgugagccccaccaagcugaacgaccugugcuucaccaacguguacgccgacagcuucgugauccggggcgacgaggugcggcagaucgcccccggccagaccggcaacaucgccgacuacaacuacaagcugcccgacgacuucaccggcugcgugaucgccuggaacagcaacaagcuggacagcaaggugagcggcaacuacaacuaccuguaccggcuguuccggaagagcaaccugaagcccuucgagcgggacaucagcaccgagaucuaccaggccggcaacaagcccugcaacggcguggccggcuucaacugcuacuucccucugcggagcuacagcuuccggcccaccuacggcgugggccaccagcccuaccgggugguggugcugagcuucgagcugcugcacgcccccgccaccgugugcggccccaagagcggcggcggcagcauccuggccaucuacagcaccguggccagcagccuggugcugcuggugagccugggcgccaucagcuucugauaauaggcuggagccucgguggccuagcuucuugccccuugggccuccccccagccccuccuccccuuccugcacccguacccccguggucuuugaauaaagucugagugggcggc

SEQ ID NO: 59 - RNA, 1563 nt, Artificial Sequence
  Synthetic
auguucguguuccuggugcugcugccccuggugagcagccagugcgugaaccugaccacccggacccagcugccuccugccuacaccaacagcuucacccggggcguguacuaccccgacaagguguuccggagcagcgugcugcacagcacccaggaccuguuccugcccuucuucagcaacgugaccugguuccacgugaucagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaacaacgccaccaacguggugaucaaggugugcgaguuccaguucugcaacgaccccuuccuggaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccuaucaucgugcgggagcccgaggaccugccccagggcuucagcgcccuggagccccugguggaccugcccaucggcaucaacaucacccgguuccagacccugcuggcccugcaccggagcuaccugaccccuggcgacagcagcagcggcuggaccgccggcgccgccgccuacuacgugggcuaccugcagccccggaccuuccugcugaaguacaacgagaacggcaccaucaccgacgccguggacggcggcggcagcggcggcggccccaacaucaccaaccugugccccuucgacgagguguucaacgccacccgguucgccagcguguacgccuggaaccggaagcggaucagcaacugcguggccgacuacagcgugcuguacaaccuggcccccuucuucaccuucaagugcuacggcgugagccccaccaagcugaacgaccugugcuucaccaacguguacgccgacagcuucgugauccggggcgacgaggugcggcagaucgcccccggccagaccggcaacaucgccgacuacaacuacaagcugcccgacgacuucaccggcugcgugaucgccuggaacagcaacaagcuggacagcaaggugagcggcaacuacaacuaccuguaccggcuguuccggaagagcaaccugaagcccuucgagcgggacaucagcaccgagaucuaccaggccggcaacaagcccugcaacggcguggccggcuucaacugcuacuucccucugcggagcuacagcuuccggcccaccuacggcgugggccaccagcccuaccgggugguggugcugagcuucgagcugcugcacgcccccgccaccgugugcggccccaagagcggcggcggcagcauccuggccaucuacagcaccguggccagcagccuggugcugcuggugagccugggcgccaucagcuuc

SEQ ID NO: 60 - protein, 521 aa, Artificial Sequence; identical to SEQ ID NO: 57
  Synthetic

SEQ ID NO: 61 - RNA, 1739 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcguguacuaccccgacaagguguuccggagcagcgugcugcacagcacccaggaccuguuccugcccuucuucagcaacgugaccugguuccacgugaucagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaacaacgccaccaacguggugaucaaggugugcgaguuccaguucugcaacgaccccuuccuggaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaucgugcgggagcccgaggaccugccccagggcuucagcgcccuggagccccugguggaccugcccaucggcaucaacaucacccgguuccagacccugcuggcccugcaccggagcuaccugaccccaggcgacagcagcagcggcuggaccgccggcgccgccgccuacuacgugggcuaccugcagccccggaccuuccugcugaaguacaacgagaacggcaccaucaccgacgccguggacggcggcggcagcggcggcggccccaacaucaccaaccugugccccuucgacgagguguucaacgccacccgguucgccagcguguacgccuggaaccggaagcggaucagcaacugcguggccgacuacagcgugcuguacaaccuggccccuuucuucaccuucaagugcuacggcgugagccccaccaagcugaacgaccugugcuucaccaacguguacgccgacagcuucgugauccggggcgacgaggugcggcagaucgccccaggccagaccggcaacaucgccgacuacaacuacaagcugcccgacgacuucaccggcugcgugaucgccuggaacagcaacaagcuggacagcaaggugagcggcaacuacaacuaccuguaccggcuguuccggaagagcaaccugaagcccuucgagcgggacaucagcaccgagaucuaccaggccggcaacaagcccugcaacggcguggccggcuucaacugcuacuucccucugcggagcuacagcuuccggcccaccuacggcgugggccaccagcccuaccgggugguggugcugagcuucgagcugcugcacgccccagccaccgugugcggccccaagagcggcggcggcagcauccuggccaucuacagcaccguggccagcagccuggugcugcuggugagccugggcgccaucagcuuuugauaauaggcuggagccucgguggccuagcuucuugccccuugggccuccccccagccccuccuccccuuccugcacccguacccccguggucuuugaauaaagucugagugggcggc

SEQ ID NO: 62 - RNA, 1563 nt, Artificial Sequence
  Synthetic
auguucguguuccuggugcugcugccccuggugagcagccagugcgugaaccugaccacccggacccagcugccaccagccuacaccaacagcuucacccggggcguguacuaccccgacaagguguuccggagcagcgugcugcacagcacccaggaccuguuccugcccuucuucagcaacgugaccugguuccacgugaucagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaacaacgccaccaacguggugaucaaggugugcgaguuccaguucugcaacgaccccuuccuggaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaucgugcgggagcccgaggaccugccccagggcuucagcgcccuggagccccugguggaccugcccaucggcaucaacaucacccgguuccagacccugcuggcccugcaccggagcuaccugaccccaggcgacagcagcagcggcuggaccgccggcgccgccgccuacuacgugggcuaccugcagccccggaccuuccugcugaaguacaacgagaacggcaccaucaccgacgccguggacggcggcggcagcggcggcggccccaacaucaccaaccugugccccuucgacgagguguucaacgccacccgguucgccagcguguacgccuggaaccggaagcggaucagcaacugcguggccgacuacagcgugcuguacaaccuggccccuuucuucaccuucaagugcuacggcgugagccccaccaagcugaacgaccugugcuucaccaacguguacgccgacagcuucgugauccggggcgacgaggugcggcagaucgccccaggccagaccggcaacaucgccgacuacaacuacaagcugcccgacgacuucaccggcugcgugaucgccuggaacagcaacaagcuggacagcaaggugagcggcaacuacaacuaccuguaccggcuguuccggaagagcaaccugaagcccuucgagcgggacaucagcaccgagaucuaccaggccggcaacaagcccugcaacggcguggccggcuucaacugcuacuucccucugcggagcuacagcuuccggcccaccuacggcgugggccaccagcccuaccgggugguggugcugagcuucgagcugcugcacgccccagccaccgugugcggccccaagagcggcggcggcagcauccuggccaucuacagcaccguggccagcagccuggugcugcuggugagccugggcgccaucagcuuu

SEQ ID NO: 63 - protein, 521 aa, Artificial Sequence; identical to SEQ ID NO: 57
  Synthetic

SEQ ID NO: 64 - RNA, 1742 nt, Artificial Sequence
  Synthetic
gggaaauaagagagaaaagaagaguaagaagaaauauaagaccccggcgccgccaccauguucguguuccuggugcugcugccccuggugagcagccagugcgugaaccugaccacccggacccagcugccaccagccuacaccaacagcuucacccggggcguguacuaccccgacaagguguuccggagcagcgugcugcacagcacccaggaccuguuccugcccuucuucagcaacgugaccugguuccacgugaucagcggcaccaacggcaccaagcgguucgacaaccccgugcugcccuucaacgacggcguguacuucgccagcaucgagaagagcaacaucauccggggcuggaucuucggcaccacccuggacagcaagacccagagccugcugaucgugaacaacgccaccaacguggugaucaaggugugcgaguuccaguucugcaacgaccccuuccuggaccacaagaacaacaagagcuggauggagagcgaguuccggguguacagcagcgccaacaacugcaccuucgaguacgugagccagcccuuccugauggaccuggagggcaagcagggcaacuucaagaaccugcgggaguucguguucaagaacaucgacggcuacuucaagaucuacagcaagcacaccccaaucaucgugcgggagcccgaggaccugccccagggcuucagcgcccuggagccccugguggaccugcccaucggcaucaacaucacccgguuccagacccugcuggcccugcaccggagcuaccugaccccaggcgacagcagcagcggcuggaccgccggcgccgccgccuacuacgugggcuaccugcagccccggaccuuccugcugaaguacaacgagaacggcaccaucaccgacgccguggacggcggcggcagcggcggcggccccaacaucaccaaccugugccccuucgacgagguguucaacgccacccgguucgccagcguguacgccuggaaccggaagcggaucagcaacugcguggccgacuacagcgugcuguacaaccuggccccuuucuucaccuucaagugcuacggcgugagccccaccaagcugaacgaccugugcuucaccaacguguacgccgacagcuucgugauccggggcgacgaggugcggcagaucgccccaggccagaccggcaacaucgccgacuacaacuacaagcugcccgacgacuucaccggcugcgugaucgccuggaacagcaacaagcuggacagcaaggugagcggcaacuacaacuaccuguaccggcuguuccggaagagcaaccugaagcccuucgagcgggacaucagcaccgagaucuaccaggccggcaacaagcccugcaacggcguggccggcuucaacugcuacuucccucugcggagcuacagcuuccggcccaccuacggcgugggccaccagcccuaccgggugguggugcugagcuucgagcugcugcacgccccagccaccgugugcggccccaagagcggcggcggcagcauccuggccaucuacagcaccguggccagcagccuggugcugcuggugagccugggcgccaucagcuuuugauaauaggcuggagccucgguggccuagcuucuugccccuugggccuccccccagccccuccuccccuuccugcaggagugagagagaaguggucuuugaauaaagucugagugggcggc

SEQ ID NO: 65 - RNA, 122 nt, Artificial Sequence
  Synthetic
ugauaauaggcuggagccucgguggccuagcuucuugccccuugggccuccccccagccccuccuccccuuccugcaggagugagagagaaguggucuuugaauaaagucugagugggcggc

SEQ ID NO: 66 - protein, 5 aa, Artificial Sequence
  Synthetic
KPSKR

SEQ ID NO: 67 - protein, 4 aa, Artificial Sequence
  Synthetic
GGGS

(19) United States
(12) Patent Application Publication
(10) Pub. No.: US 2024/0100151 A1
(43) Pub. Date: Mar. 28, 2024

(54) VARIANT STRAIN-BASED CORONAVIRUS VACCINES

(71) Applicant: ModernaTX, Inc., Cambridge, MA (US)

(72) Inventors: Andrea Carfi, Cambridge, MA (US); Guillaume Stewart-Jones, Cambridge, MA (US); Hamilton Bennett, Cambridge, MA (US); Kai Wu, Cambridge, MA (US); Darin Edwards, Cambridge, MA (US); Gwo-Yu Chuang, Cambridge, MA (US); David Reid, Cambridge, MA (US)

(73) Assignee: Moderna TX, Inc., Cambridge, MA (US)

(21) Appl. No.: 18/272,512

(22) PCT Filed: Jan. 14, 2022
(86) PCT No.: PCT/US2022/012607

§ 371 Date: Jul. 14, 2023

Related U.S. Application Data
(60) Provisional application No. 63/284,565, filed on Nov. 30, 2021.
(60) Provisional application No. 63/283,795, filed on Nov. 29, 2021.
(60) Provisional application No. 63/241,944, filed on Sep. 8, 2021.
(60) Provisional application No. 63/222,930, filed on Jul. 16, 2021.
(60) Provisional application No. 63/193,558, filed on May 26, 2021.
(60) Provisional application No. 63/173,972, filed on Apr. 12, 2021.
(60) Provisional application No. 63/161,439, filed on Mar. 15, 2021.
(60) Provisional application No. 63/140,921, filed on Jan. 24, 2021.
(60) Provisional application No. 63/138,228, filed on Jan. 15, 2021.

(51) Int. Cl. A61K 39/215; A61K 9/127; A61P 31/14

(52) U.S. Cl. CPC A61K 39/215; A61K 9/1272; A61P 31/14; A61K 2039/53; C12N 2770/20034

(57) ABSTRACT

The disclosure provides coronavirus mRNA vaccines, including vaccines directed against one or more variant strains of SARS-CoV-2, as well as methods of using the vaccines.

### RELATED APPLICATIONS

This application claims priority under 35 U.S.C. § 119(e) to U.S. Provisional Patent Application No. 63/138,228, filed Jan. 15, 2021, U.S. Provisional Patent Application No. 63/140,921, filed Jan. 24, 2021, U.S. Provisional Patent Application No. 63/161,439, filed Mar. 15, 2021, U.S. Provisional Patent Application No. 63/173,972, filed Apr. 12, 2021, U.S. Provisional Patent Application No. 63/193,558, filed May 26, 2021, U.S. Provisional Patent Application No. 63/222,930, filed Jul. 16, 2021, U.S. Provisional Patent Application No. 63/241,944, filed Sep. 8, 2021, U.S. Provisional Patent Application No. 63/283,795, filed Nov. 29, 2021, and U.S. Provisional Patent Application No. 63/284,565, filed Nov. 30, 2021, each of which are hereby incorporated by reference in their entireties.

### BACKGROUND

Human coronaviruses are highly contagious enveloped, positive single-stranded RNA viruses of the Coronaviridae family. Two sub-families of Coronaviridae are known to cause human disease. The most important being the β-coronaviruses (betacoronaviruses). Several previously known β-coronaviruses are common etiological agents of mild to moderate upper respiratory tract infections. However, in recent years there have been successive outbreaks of more lethal coronaviruses, such as SARS and MERS. The most recent novel coronavirus was initially identified from the Chinese city of Wuhan in December 2019 and has been associated with a high mortality rate. This recently identified coronavirus, referred to as Severe Acute Respiratory Syndrome Coronavirus 2 (SARS-CoV-2) (formerly referred to as a “2019 novel coronavirus,” or a “2019-nCoV”) has rapidly infected millions of people and caused a global pandemic. The pandemic disease that the SARS-CoV-2 virus causes has been named by World Health Organization (WHO) as COVID-19 (Coronavirus Disease 2019). The first genome sequence of a SARS-CoV-2 isolate (Wuhan-Hu-1; USA-WA1/2020 isolate) was released by investigators from the Chinese CDC in Beijing on Jan. 10, 2020 at Virological, a UK-based discussion forum for analysis and interpretation of virus molecular evolution and epidemiology. The sequence was then deposited in GenBank on Jan. 12, 2020, having Genbank Accession number MN908947.1. Subsequently, a number of SARS-CoV-2 strain variants have been identified, some of which are more infectious than the SARS-CoV-2 isolate.

As of the time of worldwide emergency use authorization of the authorized SARS-CoV-2 nucleic acid-based vaccines, there is not yet a strategy for combatting the recently-discovered and later-emerging SARS-CoV-2 variants of concern (VOC). The continuing health problems and mortality associated with coronavirus infections, particularly the SARS-CoV-2 pandemic, are of tremendous concern internationally. The public health crisis caused by SARS-CoV-2 and its variants reinforces the importance of rapidly developing effective and safe vaccine candidates against these viruses.

The emergence of SARS-CoV-2 variants with substitutions in the receptor binding domain (RBD) and N-terminal domain (NTD) of the viral S protein has raised concerns among scientists and health officials. The entry of coronavirus into host cells is mediated by interaction between the RBD of the viral S protein and host angiotensin-converting enzyme 2 (ACE2). Vaccine development has focused on inducing antibody responses against this region of SARS-CoV-2 S protein. More recently, a neutralization “supersite” has also been identified in the NTD. A significant decrease in vaccine efficacy has been correlated with amino acid substitutions in the RBD (eg, K417N, E484K, and N501Y) and NTD (eg, L18F, D80A, D215G, and 4242-244) of the S protein. Some of the most recently circulating isolates containing these substitutions from the United Kingdom (B.1.1.7, Alpha), Republic of South Africa (B.1.351, Beta), Brazil (P.1 lineage, Gamma), New York (B.1.526, Iota), and California (B.1.427/B.1.429 or CAL.20C lineage, Epsilon), have shown a reduction in neutralization from convalescent serum in pseudovirus neutralization (PsVN) assays and resistance to certain monoclonal antibodies. In particular, mutations in the NTD subdomain, and specifically the neutralization supersite, are most extensive in the B.1.351 lineage virus. See McCallum, M. et al. N-terminal domain antigenic mapping reveals a site of vulnerability for SARS-CoV-2. Cell, doi:10.1016/j.cell.2021.03.028 (2021).

Using 2 orthogonal vesicular stomatitis virus (VSV) and lentivirus PsVN assays expressing S variants of 20E (EU1), 20A.EU2, D614G-N439K, mink cluster 5, B.1.1.7, P.1, B.1.427/B.1.429, B.1.1.7+E484K, and B.1.351, the assessment of the neutralizing capacity of sera from Phase 1 participants and non-human primates (NHPs) that received 2 doses of mRNA-1273 was reported. See Wu, K. et al. Serum Neutralizing Activity Elicited by mRNA-1273 Vaccine. N Engl J Med, doi:10.1056/NEJMc2102179 (2021). Subsequent studies demonstrated reduced neutralization titers against the full B.1.351 variant following mRNA-1273 vaccination, although levels are still significant and expected to be protective. Despite this prediction of continued efficacy of mRNA-1273 against this key variant of concern, the duration of vaccine mediated protection is still unknown.

There remains a need for development and evaluation of further COVID-19 vaccines against SARS-CoV-2 variants encoding the prefusion stabilized S protein of SARS-CoV-2 that incorporates key mutations present in the variants, including L18F, D80A, D215G, L242-244del, R246I, K417N, E484K, N501Y, D614G, A701V, A67V, Δ69-70, T95I, G142D/Δ143-145, Δ211/L212I, ins214EPE, G339D, S371L, S373P, S375F, N440K, G446S, S477N, T478K, E484A, Q493K, G496S, Q498R, Y505H, T547K, H655Y, N679K, P681H, N764K, D796Y, N856K, Q954H, N969K, L981F, and any combination thereof. Additional vaccines are necessary to expand the breadth of coverage to multiple circulating variants as well as the ancestral wild-type virus that is still circulating globally.

### SUMMARY

A SARS-CoV-2 vaccine, mRNA-1273 (developed by Moderna Therapeutics), has been shown to elicit high viral neutralizing titers in Phase 1 trial human participants (Jackson et al, 2020; Anderson et al, 2020) and is highly efficacious in prevention of symptomatic COVID-19 disease and severe disease (Baden et al., 2020). However, the recent emergence of SARS-CoV-2 variants in the United Kingdom (B.1.1.7 lineage; alpha variant) and in South Africa (B.1.351 lineage; beta variant) have raised concerns due to their increased rates of transmission as well as their potential to circumvent immunity elicited by natural infection or vaccination (Volz et al., 2021; Tegally et al., 2020; Wibmer et al., 2021; Wang et al., 2021; Collier et al., 2021).

First detected in September 2020 in South England, the SARS-CoV-2 B.1.1.7 variant (alpha variant) has spread at a rapid rate and is associated with increased transmission and higher viral burden (Rambaut et al., 2020). This variant has seventeen mutations in the viral genome. Among them, eight mutations are located in the spike (S) protein, including 69-70 del, Y144 del, N501Y, A570D, P681H, T716I, S982A and D1118H. Two key features of this variant, the 69-70 deletion and the N501Y mutation in S protein, have generated concern among scientists and policy makers in the UK based on increased transmission and potentially increased mortality, resulting in further shutdowns. The 69-70 deletion is associated with reduced sensitivity to neutralization by SARS-CoV-2 human convalescent serum samples (Kemp et al, 2021). N501 is one of the six key amino acids interacting with ACE-2 receptor (Starr et al. 2020), and the tyrosine substitution has been shown to have increased binding affinity to the ACE-2 receptor (Chan et al., 2020).

The B.1.351 variant (beta variant) emerged in South Africa over the past few months, and, similar to the B.1.1.7 variant, increased rates of transmission and higher viral burden after infection have been reported (Tegally et al., 2020). The mutations located in the S protein are more extensive than the B.1.1.7 variant with changes of L18F, D80A, D215G, L242-244del, R246I, K417N, E484K, N501Y, D614G, and A701V, with three of these mutations located in the RBD (K417N, E484K, N501Y). B.1.351 shares key mutations in the RBD with a reported variant in Brazil (Tegally et al., 2020; Naveca et al., 2021). As the RBD is the predominant target for neutralizing antibodies, these mutations could impact the effectiveness of monoclonal antibodies already approved and in advanced development as well as of polyclonal antibody elicited by infection or vaccination in neutralizing the virus (Greaney et al., 2021, Wibmer et al, 2021).

Recent data have suggested that the key mutation present in the B.1.351 variant, E484K, confers resistance to SARS-CoV-2 neutralizing antibodies, potentially limiting the therapeutic effectiveness of monoclonal antibody therapies (Wang et al., 2021; Greaney et al., 2020; Weisblum et al., 2020; Liu et al., 2020; Wibmer et al., 2021). Moreover, the E484K mutation was shown to reduce neutralization against a panel of convalescent sera (Weisblum et al., 2020; Liu et al., 2020; Wibmer et al., 2021). In terms of vaccination, it is clear that mRNA-1273 induces significantly higher neutralizing titers than convalescent sera against the USA-WA1/2020 isolate (Jackson et al, 2020). A recent study using a recombinant VSV PsVN assay showed that sera of mRNA-1273 vaccinated participants had reduced neutralizing titers against E484K or K417N/E484K/N50Y combination (Wang et al, 2021), however there has been no assessment of sera from mRNA-1273 clinical trial participants against the full constellation of S mutations found in the B.1.1.7 or B.1.351 variants.

Neutralization of sera from mRNA-1273 vaccinated Phase 1 clinical trial participants against recombinant VSV-based SARS-CoV-2 PsVN assay with S protein from the USA-WA1/2020 isolate, D614G variant, the B.1.1.7 and B.1.351 variants, and variants that have previously emerged (20E, 20A.EU2, D614G-N439K, and mink cluster 5 variant) was examined (data discussed in the Examples). The effect of both single mutations and combinations of mutations present in the RBD region of the S protein was assessed. In addition, orthogonal assessments in VSV and pseudotyped lentiviral neutralization assays were performed on sera from NHPs that received the mRNA-1273 vaccine at two different dose levels, as this has been a useful pre-clinical model for vaccine induced immunogenicity and protection. Using both of these assays provided confirmatory data on pseudovirus neutralization. Overall, this comprehensive pseudovirus neutralization analysis in humans and non-human primates that received mRNA-1273 provides a critical demonstration necessary to elucidate how vaccines may be impacted by SARS-CoV-2 variants.

The invention pertains, inter alia, to vaccines comprising a nucleic acid encoding a SARS-CoV-2 antigen, which varies by at least one amino acid mutation from the SARS-CoV-2 spike antigen having an amino acid sequence of SEQ ID NO: 36 wherein the mutation is an amino acid substitution, deletion or insertion, wherein if the mutation is an insertion it is not a 2P mutation or is in addition to a 2P mutation. Such a vaccine, optionally referred to herein as a variant vaccine, can be administered to seropositive or seronegative subjects. For example, a subject may be naïve and not have antibodies that react with SARS-CoV-2 or may have preexisting antibodies to SARS-CoV-2 because they have previously had an infection with SARS-CoV-2 or may have previously been administered a dose of a vaccine (e.g., an mRNA vaccine) that induces antibodies against SARS-CoV-2. A variant vaccine may be the only vaccine comprising a nucleic acid encoding a SARS-CoV-2 antigen that a subject receives. Alternatively a variant vaccine may be administered in combination with other vaccines comprising a nucleic acid encoding a SARS-CoV-2 antigen, as a prime and/or boost dose.

Thus, the disclosure, in some aspects provides a method comprising administering to a subject a vaccine, e.g., an effective dose thereof, comprising a nucleic acid encoding a SARS-CoV-2 spike antigen, optionally a 2P stabilized spike antigen of a second circulating SARS-CoV-2 virus, wherein the subject has previously been administered a first vaccine comprising a nucleic acid encoding a first SARS-CoV-2 2P stabilized spike antigen of a first circulating SARS-CoV-2 virus, and wherein each of the first and second 2P stabilized spike antigens are administered in an effective amount to induce an immune response specific for the first antigen and the second antigen, wherein the second circulating SARS-CoV-2 virus has a spike protein having an amino acid sequence with at least one amino acid mutation with respect to a spike protein amino acid sequence of the first circulating SARS-CoV-2 virus, and wherein the mutation is an amino acid substitution, deletion or insertion.

In some aspects, the disclosure provides a method comprising administering to a subject a first vaccine, e.g., an effective dose thereof, comprising a nucleic acid encoding a first SARS-CoV-2 2P stabilized spike antigen and administering to the subject a second vaccine comprising a nucleic acid encoding a second SARS-CoV-2 2P spike antigen, optionally a 2P stabilized spike antigen, wherein each of the nucleic acids encoding the first and second stabilized spike antigens are administered in an effective amount to induce an immune response specific for the respective encoded antigens, wherein the second encoded SARS-CoV-2 spike antigen has an amino acid sequence with at least one amino acid mutation with respect to the first encoded spike protein amino acid sequence, and wherein the mutation is an amino acid substitution, deletion, or insertion.

In another aspect, the disclosure provides a method comprising administering to a subject a first vaccine, e.g., an effective dose thereof, comprising a nucleic acid encoding a first SARS-CoV-2 2P stabilized spike antigen and administering to the subject a second vaccine comprising a nucleic acid encoding a second SARS-CoV-2 2P spike antigen, optionally a 2P stabilized spike antigen, wherein each of the nucleic acids encoding the first and second stabilized spike antigens are administered in an effective amount to induce an immune response specific for the respective encoded antigens, wherein the second encoded SARS-CoV-2 spike antigen has an amino acid sequence with at least one amino acid mutation with respect to the first encoded spike protein amino acid sequence, wherein the mutation is an amino acid substitution, deletion, or insertion, and wherein the first encoded SARS-CoV-2 spike antigen is of a first circulating SARS-CoV-2 virus and wherein the second encoded SARS-CoV-2 spike antigen is of a second circulating SARS-CoV-2 virus.

Provided herein, in some aspects, are compositions (e.g., vaccines) that comprise one or more messenger ribonucleic acid (mRNA) that encode(s) highly immunogenic antigen(s) capable of eliciting potent neutralizing antibody responses against coronavirus antigens such as SARS-CoV-2 variant antigens.

The disclosure provides, in some aspects, a nucleic acid (e.g., messenger ribonucleic acid (mRNA)) encoding a SARS-CoV-2 antigen of a second circulating SARS-CoV-2 virus strain, wherein the antigen has an amino acid sequence with at least one amino acid mutation with respect to an antigen of a first circulating SARS-CoV-2 virus strain, wherein the mutation is an amino acid substitution, deletion or insertion, wherein the antigen is not a full length stabilized spike protein having a 2P mutation and wherein the mRNA is in a lipid nanoparticle.

In some embodiments, the first and second virus strains are in circulation for at least a portion of 1 year. In some embodiments, the first and second virus strains are in circulation during the same pandemic or endemic.

In some embodiments, the SARS-CoV-2 antigen has a second amino acid mutation, deletion, or both amino acid mutation and deletion with respect to the antigen of the first circulating SARS-CoV-2 virus strain and the second mutation or deletion corresponds to a third virus strain in circulation.

In some embodiments, the lipid nanoparticle comprises an ionizable amino lipid, a sterol, a neutral lipid, and a polyethylene glycol (PEG)-modified lipid. In some embodiments, the lipid nanoparticle comprises 40-55 mol % ionizable amino lipid, 30-45 mol % sterol, 5-15 mol % neutral lipid, and 1-5 mol % PEG-modified lipid.

In some embodiments, the SARS-CoV-2 antigen is a receptor binding domain (RBD) of spike protein. In some embodiments, the SARS-CoV-2 antigen is an N-terminal domain (NTD). In some embodiments, the SARS-CoV-2 antigen is a combination of an RBD and NTD. In some embodiments, the SARS-CoV-2 antigen is a combination of an RBD and NTD and transmembrane domain joined by linkers.

In some embodiments, the SARS-CoV-2 antigen is an NTD, RBD, and influenza hemagglutinin transmembrane (HATM) domain joined by linkers (NTD-RBD-HATM). In some embodiments, the SARS-CoV-2 antigen is an NTD-RBD fusion.

In some embodiments, the SARS-CoV-2 antigen is an S1 protein subunit. In some embodiments, the SARS-CoV-2 antigen is an S1 protein subunit.

In some embodiments, the amino acid mutation, deletion, or both amino acid mutation and deletion in the antigen is associated with a structure or function of the virus that is different in quantity or kind from a corresponding structure or function in the first virus. In some embodiments, the function is ACE2 receptor binding, virus transmissibility, viral uptake, viral pathogenesis, altered furin cleavage, or viral replication.

In some embodiments, the mRNA encodes a protein having at least 95% sequence identity to a protein of any one of SEQ ID NOs: 20, 23, 26, 29, 32, 57, 60, and 63. In some embodiments, the mRNA has at least 95% sequence identity to an RNA of any one of SEQ ID NOs: 21, 24, 27, 30, 55, 58, 61, and 64.

In some aspects, the disclosure provides a composition comprising a first messenger ribonucleic acid (mRNA) encoding a first SARS-CoV-2 antigen of a first SARS-CoV-2 virus and a second mRNA encoding a second SARS-CoV-2 antigen of a second SARS-CoV-2 virus, wherein the second SARS-CoV-2 virus has an amino acid sequence with at least one amino acid mutation, deletion, or both amino acid mutation and deletion with respect to an amino acid sequence of the first SARS-CoV-2 virus, wherein each of the first and second antigens are not full length stabilized spike proteins having a 2P mutation.

In some embodiments, the composition further comprises a third messenger ribonucleic acid (mRNA) encoding a third SARS-CoV-2 antigen of a third SARS-CoV-2 virus, wherein the third SARS-CoV-2 antigen has an amino acid sequence with at least one amino acid mutation, deletion, or both amino acid mutation and deletion with respect to an amino acid sequence of the first and second SARS-CoV-2 antigens.

In some embodiments, the composition further comprises a fourth messenger ribonucleic acid (mRNA) encoding a fourth SARS-CoV-2 antigen of a fourth SARS-CoV-2 virus, wherein the fourth SARS-CoV-2 antigen has an amino acid sequence with at least one amino acid mutation, deletion, or both amino acid mutation and deletion with respect to an amino acid sequence of the first, second, and third SARS-CoV-2 antigens.

In some embodiments, the composition further comprises a fifth messenger ribonucleic acid (mRNA) encoding a fifth SARS-CoV-2 antigen of a fifth SARS-CoV-2 virus, wherein the fifth SARS-CoV-2 antigen has an amino acid sequence with at least one amino acid mutation, deletion, or both amino acid mutation and deletion with respect to an amino acid sequence of the first, second, third and fourth SARS-CoV-2 antigens.

In some embodiments, the composition further comprises a sixth messenger ribonucleic acid (mRNA) encoding a sixth SARS-CoV-2 antigen of a sixth SARS-CoV-2 virus, wherein the sixth SARS-CoV-2 antigen has an amino acid sequence with at least one amino acid mutation, deletion, or both amino acid mutation and deletion with respect to an amino acid sequence of the first, second, third, fourth and fifth SARS-CoV-2 antigens.

In some embodiments, the first antigen and second antigen, and optionally the third, fourth, fifth and sixth antigen are each antigens of the spike protein. In some embodiments, the first antigen and second antigen, and optionally the third, fourth, fifth and sixth antigen are each antigens of the receptor binding domain of the spike protein.

In some embodiments, the first antigen and second antigen, and optionally the third, fourth, fifth and sixth antigen, each share at least 98% amino acid sequence identity with one another.

In some embodiments, the first SARS-CoV-2 virus is a first circulating SARS-CoV-2 virus strain, and the second SARS-CoV-2 virus is a second circulating SARS-CoV-2 virus strain, and wherein the first and second virus strains are spreading in the population for at least a portion of 1 year.

In some embodiments, the mRNAs are present at about a 1:1 ratio relative to each other mRNA in the composition. In other embodiments the vaccines are separate vaccines that are not co-formulated.

In some embodiments, the mRNAs are in a lipid nanoparticle and wherein the lipid nanoparticle comprises an ionizable amino lipid, a sterol, a neutral lipid, and a polyethylene glycol (PEG)-modified lipid. In some embodiments, the lipid nanoparticle comprises 40-55 mol % ionizable amino lipid, 30-45 mol % sterol, 5-15 mol % neutral lipid, and 1-5 mol % PEG-modified lipid. In some embodiments, the lipid nanoparticle comprises 40-50 mol % ionizable amino lipid, 35-45 mol % sterol, 10-15 mol % neutral lipid, and 2-4 mol % PEG-modified lipid. In some embodiments, the lipid nanoparticle comprises 45 mol %, 46 mol %, 47 mol %, 48 mol %, 49 mol %, or 50 mol % ionizable amino lipid.

In some embodiments, the ionizable amino lipid has the structure of Compound 1:

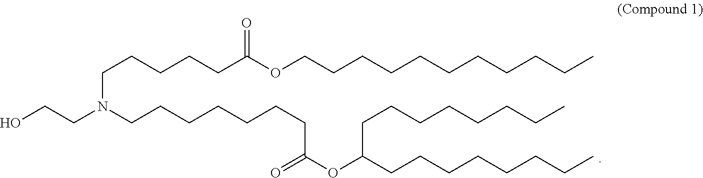 [SMILES: CCCCCCCCCCCOC(=O)CCCCCN(CCO)CCCCCCCC(=O)OC(CCCCCCCC)CCCCCCCC]

In some embodiments, the sterol is cholesterol. In some embodiments, the neutral lipid is 1,2 distearoyl-sn-glycero-3-phosphocholine (DSPC). In some embodiments, the PEG-modified lipid is 1,2 dimyristoyl-sn-glycerol, methoxypolyethyleneglycol (PEG2000 DMG).

The disclosure provides, in some aspects, an mRNA encoding a protein having at least 90% or 95% sequence identity to a protein of any one of SEQ ID NOs: 20, 23, 26, 29, 32, 57, 60, and 63.

In some aspects, the disclosure provides an mRNA having at least 90% or 95% sequence identity to an RNA of any one of SEQ ID NOs: 21, 24, 27, 30, 55, 58, 61, and 64.

In another aspect, the disclosure provides an mRNA having at least 98% sequence identity to an RNA of any one of SEQ ID NOs: 21, 24, 27, 30, 55, 58, 61, and 64.

In some aspects, the disclosure provides an mRNA comprising an RNA of any one of SEQ ID NOs: 21, 24, 27, 30, 55, 58, 61, and 64.

In some aspects, the disclosure provides a method comprising: administering to a subject a first effective dose of a first SARS-CoV-2 antigen of a first circulating SARS-CoV-2 virus and administering to the subject a second effective dose of a nucleic acid encoding a second SARS-CoV-2 antigen of a second circulating SARS-CoV-2 virus, wherein the first and second antigens are administered in an effective amount to induce an immune response specific for the first antigen and the second antigen, wherein the second SARS-CoV-2 virus has an amino acid sequence with at least one amino acid mutation, deletion, or both amino acid mutation and deletion with respect to an amino acid sequence of the first SARS-CoV-2 virus, and wherein each of the first and second antigens are not full length stabilized spike proteins having a 2P mutation.

In some embodiments, the second circulating SARS-CoV-2 virus is an immunodominant emerging strain detected during a period when the first circulating SARS-CoV-2 virus is present in a subject population.

In some embodiments, the second circulating SARS-CoV-2 virus and the first circulating SARS-CoV-2 virus are detectable in a subject population within at least one year. In some embodiments, the second circulating SARS-CoV-2 virus and the first circulating SARS-CoV-2 virus are detectable in a subject population during a same season. In some embodiments, the second circulating SARS-CoV-2 virus and the first circulating SARS-CoV-2 virus are detectable in a subject population during a same pandemic or endemic.

In some embodiments, the first SARS-CoV-2 antigen is a first nucleic acid encoding the first SARS-CoV-2 antigen. In some embodiments, the first nucleic acid is a DNA or a messenger RNA (mRNA). In some embodiments, the second vaccine further comprises the first nucleic acid encoding a first SARS-CoV-2 antigen. In some embodiments, the first nucleic acid encoding a first SARS-CoV-2 antigen and the second nucleic acid encoding a second SARS-CoV-2 antigen are present in the second vaccine in a 1:1 ratio.

In some embodiments, the nucleic acid encoding a second SARS-CoV-2 antigen of a second circulating SARS-CoV-2 virus is s second nucleic acid and is a messenger RNA (mRNA).

In some embodiments, the first and/or second spike antigen is a monovalent antigen.

In some embodiments, the first and/or second spike antigen is a multivalent antigen.

In some embodiments, the first encoded antigen is administered to the subject as a first vaccine comprised of one or more prime or priming immunization and the second encoded antigen is administered to the subject as a boost. In some embodiments, the first and second encoded SARS-CoV-2 antigens are present in the boost dose in a 1:1 ratio.

In some embodiments, the second encoded antigen is administered to the subject as first vaccine comprised of one or more prime or priming immunizations and the first encoded antigen is administered to the subject as a boost.

In some embodiments, the first and second encoded antigens are administered to the subject together as a boost.

In some embodiments, the first encoded antigen is administered to the subject as a prime or priming immunization and as a boost to complete a vaccination.

In some embodiments, the first encoded antigen is administered to the subject as a prime or priming immunization and as a boost in an initial vaccination and the second encoded antigen is administered to the subject as a boost more than 3 months after the initial vaccination.

In some embodiments, the boost is a seasonal boost or a pandemic shift boost.

The disclosure provides, in some aspects, a method comprising administering to the subject a second effective dose of a second vaccine comprising a second nucleic acid encoding a second SARS-CoV-2 antigen, wherein the subject has previously been administered a first effective dose of a first vaccine comprising a first nucleic acid encoding a first SARS-CoV-2 antigen, wherein the second vaccine is administered in an effective amount to induce an immune response specific for the second antigen, wherein the second SARS-CoV-2 antigen has an amino acid sequence with at least one mutation, with respect to a corresponding amino acid sequence of the first SARS-CoV-2 antigen, wherein the mutation is an amino acid substitution, deletion or insertion and wherein each of the first and second antigens are not full length stabilized spike proteins having a 2P mutation.

In some embodiments, the first encoded SARS-CoV-2 antigen is of a first circulating SARS-CoV-2 virus and wherein the second encoded SARS-CoV-2 antigen is of a second circulating SARS-CoV-2 virus. In some embodiments, the first encoded SARS-CoV-2 antigen is representative of a first circulating SARS-CoV-2 virus and wherein the second encoded SARS-CoV-2 antigen is representative of a second circulating SARS-CoV-2 virus.

In some embodiments, the first encoded SARS-CoV-2 antigen is representative of a plurality of first circulating SARS-CoV-2 viruses and/or wherein the second encoded SARS-CoV-2 antigen is representative of a second plurality of circulating SARS-CoV-2 viruses.

In some embodiments, the first encoded SARS-CoV-2 antigen is of a first circulating SARS-CoV-2 virus and wherein the second encoded SARS-CoV-2 antigen is of a second circulating SARS-CoV-2 virus.

In some embodiments, the second circulating SARS-CoV-2 virus is an immunodominant emerging strain or variant of concern detected during a period when the first circulating SARS-CoV-2 virus is present in a subject population. In some embodiments, the second circulating SARS-CoV-2 virus and the first circulating SARS-CoV-2 virus are detectable in a subject population within at least one year. In some embodiments, the second circulating SARS-CoV-2 virus and the first circulating SARS-CoV-2 virus are detectable in a subject population during a same season. In some embodiments, the second circulating SARS-CoV-2 virus and the first circulating SARS-CoV-2 virus are detectable in a subject population during a same pandemic period or endemic period.

In some embodiments, the first nucleic acid encoding the first SARS-CoV-2 antigen is a DNA or a messenger RNA (mRNA). In some embodiments, the first nucleic acid encoding a first SARS-CoV-2 antigen is a messenger RNA (mRNA). In some embodiments, the second nucleic acid encoding a second SARS-CoV-2 antigen is a messenger RNA (mRNA). In some embodiments, the second vaccine further comprises the first nucleic acid encoding a first SARS-CoV-2 antigen. In some embodiments, the first nucleic acid encoding a first SARS-CoV-2 antigen and the second nucleic acid encoding a second SARS-CoV-2 antigen are present in the second vaccine in a 1:1 ratio.

In some embodiments, the first and/or second vaccine is a monovalent vaccine. In some embodiments, the first and/or second vaccine is a multivalent vaccine, comprising more than one nucleic acids encoding antigens representative of a plurality of variants of concern.

In some embodiments, the first encoded SARS-CoV-2 antigen is administered to the subject as a first vaccine comprised of one or more prime or priming immunizations and the second encoded SARS-CoV-2 antigen is administered to the subject as a boost in one or more administrations.

In some embodiments, the second encoded SARS-CoV-2 antigen is administered to the subject as first vaccine comprised of one or more prime or priming immunizations and the first encoded SARS-CoV-2 antigen is administered to the subject as in one or more boosts

In some embodiments, the first and second encoded SARS-CoV-2 antigens are administered to the subject together as a boost. In some embodiments, the first and second encoded SARS-CoV-2 antigens are present in the boost dose in a 1:1 ratio.

In some embodiments, the first encoded SARS-CoV-2 antigen is administered to the subject as a prime or priming immunization and as a boost to complete a vaccination and wherein the second encoded SARS-CoV-2 antigen is administered to the subject as a booster regimen at least 3 months after the first vaccination is complete.

In some embodiments, the first encoded SARS-CoV-2 antigen is administered to the subject as a prime or priming immunization and as a boost in an initial vaccination and the second encoded SARS-CoV-2 antigen is administered to the subject as a boost more than 6 months after the initial vaccination.

In some embodiments, the boost is a seasonal boost or a pandemic shift boost to provide protection for a plurality of variants of concern.

In some embodiments, the boost dose is 50 μg.

In some embodiments, the first and/or the second effective dose is 20 μg-50 μg. In some embodiments, the first and/or the second effective dose is 20 μg. In some embodiments, the first and/or the second effective dose is 25 μg. In some embodiments, the first and/or the second effective dose is 30 μg. In some embodiments, the first and/or the second effective dose is 40 μg. In some embodiments, the first and/or the second effective dose is 50 μg. In some embodiments, the first and/or second effective dose is 30 μg. In some embodiments, the first and/or second effective dose is a 10 μg. In some embodiments, the first and/or second effective dose is 10 μg-30 μg. In some embodiments, the first and/or second effective dose is 10 μg-20 μg. In some embodiments, the first and/or second effective dose is at least 10 μg and less than 25 μg. In some embodiments, the first and/or second effective dose is 5 μg-30 μg. In some embodiments, the first and/or second effective dose is at least 5 μg and less than 25 μg.

In some aspects, the disclosure provides a method comprising administering to a subject a vaccine comprising a first nucleic acid encoding a SARS-CoV-2 antigen of a first SARS-CoV-2 virus, wherein the first SARS-CoV-2 virus is an emerging variant of a primary SARS-CoV-2 virus, wherein the antigen has an amino acid sequence with at least one amino acid mutation with respect to a corresponding protein antigen of the primary SARS-CoV-2 virus, and wherein the mutation is an amino acid substitution, deletion or insertion, and wherein the subject is seropositive for a SARS-CoV-2 antigen of the primary SARS-CoV-2 virus.

The disclosure also provides a method comprising administering to a subject a vaccine comprising a first nucleic acid encoding a SARS-CoV-2 antigen of a first SARS-CoV-2 virus, wherein the first SARS-CoV-2 virus is an emerging variant of a primary SARS-CoV-2 virus, wherein the antigen has an amino acid sequence with at least one amino acid mutation with respect to a corresponding protein antigen of the primary SARS-CoV-2 virus, and wherein the mutation is an amino acid substitution, deletion or insertion, and wherein the subject is seronegative for a SARS-CoV-2 antigen of the primary SARS-CoV-2 virus.

In some embodiments, the subject is administered a second dose of the vaccine between 2 weeks and 1 year after the first dose of vaccine is administered. In some embodiments, the subject is administered a second vaccine between 2 weeks and 1 year after the vaccine is administered, wherein the second vaccine comprises a second nucleic acid encoding the corresponding protein antigen of the primary SARS-CoV-2 virus. In some embodiments, the second vaccine comprises a mixture of the first and second nucleic acids, wherein the first nucleic acid and the second nucleic acid are present in the second vaccine at a ratio of 1:1.

In some aspects, the disclosure provides a method comprising administering to a subject a booster vaccine comprising a nucleic acid encoding a first SARS-CoV-2 antigen from a first SARS-CoV-2 virus, wherein the subject has previously been administered at least one prime dose of a first vaccine comprising a first nucleic acid encoding the SARS-CoV-2 antigen of the first the SARS-CoV-2 virus, wherein the booster vaccine is administered in an effective amount to induce a neutralizing immune response against a second SARS-CoV-2 virus, wherein the second SARS-CoV-2 virus comprises a second SARS-CoV-2 antigen, wherein the second SARS-CoV-2 antigen has an amino acid sequence with at least one amino acid mutation with respect to a corresponding protein antigen of the first SARS-CoV-2 virus, wherein the booster vaccine is administered in a dosage of 25-100 μg at least 6 months after a first dose of the first vaccine, and wherein each of the first and second antigens are not full length stabilized spike proteins having a 2P mutation.

In some embodiments, the booster vaccine is administered in a dosage of 50 μg.

In some embodiments, the booster vaccine is administered at least about 6 months after a second dose of the first vaccine. In some embodiments, the booster vaccine is administered 6-12 months after a second dose of the first vaccine. In some embodiments, the booster vaccine is administered at least about 8 months after a second dose of the first vaccine.

In some embodiments, the boost dose is a seasonal boost or a pandemic shift boost to provide a neutralizing immune response against a plurality of variants of concern.

### BRIEF DESCRIPTION OF THE DRAWINGS

FIG. 1 shows neutralization titers of murine serum samples. Serum was from mice immunized with 1 μg of NTD-RBD-HATM. The virus being neutralized contained either the D614G mutation or the mutations associated with the South Africa Variant B.1.351.

FIG. 2 shows neutralization titers of murine serum samples. Serum was from mice administering 1 μg of NTD_ext-RBD_ext-TM. The virus being neutralized contained either the D614G mutation or the mutations associated with the South Africa Variant B.1.351.

FIG. 3 shows neutralization titers of murine serum samples. Serum was from mice administering 1 μg of RBD_WH2020_NatSP_317_516_TM. The virus being neutralized contained either the D614G mutation or the mutations associated with the South Africa Variant B.1.351.

FIGS. 4A-4C show neutralization titers of murine serum samples. Sera was from mice administered 1 μg of NTD-RBD-HATM on days 1 and 22 and then 1 μg of NTD-RBD-HATM comprising the mutations associated with the South Africa variant, B.1.351 on day 213. Samples were taken on day 212 (before administration of the third dose) and day 233 (after administration of the third dose). FIG. 4A shows the neutralizing antibody titer at each time point. The virus being neutralized contained either the D614G mutation or the mutations associated with the South Africa Variant B.1.351. FIG. 4B shows the relative titer change between the two time points and FIG. 4C shows the relative titer change between the different variants at the two time points.

FIGS. 5A-5C show neutralization titers of murine serum samples. Sera was from mice administered 0.1 μg of NTD-RBD-HATM on days 1 and 22 and then 0.1 μg of NTD-RBD-HATM comprising the mutations associated with the South Africa variant, B.1.351 on day 213. Samples were taken on day 212 (before administration of the third dose) and day 233 (after administration of the third dose). FIG. 5A shows the neutralizing antibody titer at each time point. The virus being neutralized contained either the D614G mutation or the mutations associated with the South Africa Variant B.1.351. FIG. 5B shows the relative titer change between the two time points and FIG. 5C shows the relative titer change between the different variants at the two time points.

FIGS. 6A-6D show neutralization titers of murine serum samples. Sera was obtained from mice that were administered 1 μg of NTD-RBD-HATM on day 1 (prime dose) and again on day 22 (booster dose). Mice were administered 1 μg of NTD-RBD-HATM comprising the mutations associated with the South Africa variant, B.1.351, (mRNA-1283.351) on day 213 (3^{rd} dose), and day 234 (4^{th} dose). Samples were taken at day 212 (before administration of the 3^{rd} dose), day 233 (before administration of the 4^{th} dose), and day 248 (14 days after administration of the 4^{th} dose). FIG. 6A shows the neutralizing antibody titer at each time point, based on neutralization of pseudoviruses comprising a SARS-CoV-2 Spike protein with a D614G mutation (left bars) or the mutations associated with the South Africa B.1.351 variant. FIG. 6B shows, for each Spike protein tested in FIG. 6A, the kinetics of neutralization titers from day 212 through day 248. FIG. 6C shows, at each time point, the relative change in neutralization titers towards a B.1.351 Spike protein compared to a Spike protein comprising only a D614G mutation. FIG. 6D shows reference neutralization titers of sera from day 36, two weeks after a second dose, towards D614G Spike protein.

FIGS. 7A-7D show neutralization titers of murine serum samples. Sera was obtained from mice that were administered 0.1 μg of NTD-RBD-HATM on day 1 (prime dose) and again day 22 (booster dose). Mice were administered 0.1 μg of NTD-RBD-HATM comprising the mutations associated with the South Africa variant, B.1.351, (mRNA-1283.351) on day 213 (3^{rd} dose), and day 234 (4^{th} dose). Samples were taken at day 212 (before administration of the 3^{rd} dose with mRNA-1283.351), day 233 (before administration of the 4^{th} dose with mRNA-1283.351), and day 248 (14 days after administration of the 4^{th} dose with mRNA-1283.351). FIG. 7A shows the neutralizing antibody titer at each time point, based on neutralization of pseudoviruses comprising a SARS-CoV-2 Spike protein with a D614G mutation (left bars) or the mutations associated with the South Africa B.1.351 variant. FIG. 7B shows, for each Spike protein tested in FIG. 7A, the kinetics of neutralization titers from day 212 through day 248. FIG. 7C shows, at each time point, the relative change in neutralization titers towards a B.1.351 Spike protein compared to a Spike protein comprising only a D614G mutation. FIG. 7D shows reference neutralization titers of sera from day 36, two weeks after a second dose, towards D614G Spike protein.

FIGS. 8A-8D show protein-specific IgG and neutralization titers of murine serum samples. Sera were collected from mice administered 10, 1, or 0.1 μg NTD-ext-RBD-ext-HATM on day 1 (prime dose), and again on day 22 (2^{nd} dose). Sera were collected at day 36 (14 days after 2^{nd} dose). FIG. 8A shows total IgG specific to SARS-CoV-2 Spike protein, as measured by ELISA. FIG. 8B shows neutralization titers of serum samples towards pseudoviruses containing one of a panel of Spike proteins, including 1) a Spike protein with a D614G mutation, 2) a Spike protein with the mutations associated with the B.1.351 variant. 3) a Spike protein with the mutations associated with the P.1 variant, and 4) a Spike protein with the mutations associated with the B.1.1.7 variant as well as an E484K mutation. FIG. 8C shows the reduction in neutralization titers for sera against each of the viruses tested in FIG. 8B, relative to the baseline of neutralization titers towards D614G Spike protein. FIG. 8D shows reference neutralization titers from mice immunized with control mRNA-1273 towards each pseudovirus and Spike protein tested in FIGS. 8B-8C, as well as a pseudovirus comprising the mutations associated with the B.1.1.7 variant, but not an E484K mutation, of sera elicited by two doses (day 1 and day 14) with 1 μg of an mRNA encoding a 2P-stabilized SARS-CoV-2 Spike protein.

### DETAILED DESCRIPTION

Severe acute respiratory syndrome coronavirus 2 (SARS-CoV-2) is a newly emerging respiratory virus with high morbidity and mortality. SARS-CoV-2 has rapidly spread around the world compared with SARS-CoV, which appeared in 2002, and Middle East respiratory syndrome coronavirus (MERS-CoV), which emerged in 2012. The World Health Organization (WHO) reports that, as of March 2021, the current outbreak of COVID-19 has had over 120 million confirmed cases worldwide with more than 2.65 million deaths. New cases of COVID-19 infection are on the rise and are still increasing rapidly. It is thus crucial that a variety of safe and effective vaccines and drugs be developed to prevent and treat COVID-19 and reduce the serious impact that COVID-19 is having across the world. Vaccines and drugs made using a variety of modalities, and vaccines having improved safety and efficacy, are imperative. There remains a need to accelerate the advanced design and development of vaccines and therapeutic drugs against coronavirus disease and in particular coronavirus 2019 (COVID-19).

On Jan. 7, 2020, severe acute respiratory syndrome coronavirus 2 (SARS-CoV-2) was identified as the etiological agent of a novel pneumonia that emerged in December 2019, in Wuhan City, Hubei province in China (Lu H. et al. (2020) J Med Virol. April; 92(4):401-402.). Soon after, the virus caused an outbreak in China and has spread to the world. According to the analysis of genomic structure of SARS-CoV-2, it belongs to β-coronaviruses (CoVs) (Chan et al. 2020 Emerg Microbes Infect.; 9(1):221-236).

Subsequently, a number of SARS-CoV-2 variant strains have emerged and have predominated in particular initial geographic areas. However, some variants that quickly predominate in one geographic area can spread rapidly around the globe. These variants are known as variants of concern (VOC). Two main variants have been found since the fall of 2020, including one in the United Kingdom (20B/501Y.V1, VOC 202012/01, or B.1.1.7 lineage, or alpha variant) and one in South Africa (20C/501Y.V2 or B.1.351 lineage, or beta variant). The two variants emerged separately from one another, but appear to have improved transmissibility relative to the USA-WA1/2020 isolate. Further, there are concerns that these variants as well as other circulating strains and any future variants may further mutate to avoid neutralization by existing vaccines and therapeutic modalities such as antibodies. In this way, the SARS-CoV-2 variants, and any other emerging mutant SARS-CoV-2 strains, are an international health concern.

The threat of emerging mutant strains of viruses presents a significant challenge to vaccine development. The compositions disclosed herein provide a significant advance in combatting the emerging viral strains that pose a global health concern. Disclosed herein are vaccines and vaccine protocols with broad viral neutralization capabilities that reduce the threat of infection from more than one strain of virus, through single or multiple administrations of the same or different combinations of antigens from different strains. For instance, the vaccination strategies disclosed herein, in some embodiments, comprise “primary series” of vaccinations and subsequent boost(s) of SARS-CoV-2 antigen. The primary series (also referred to herein as initial, original or first vaccine, or vaccination) involves the administration of one or more doses (e.g. two doses) of the SARS-CoV-2 antigen from the originally identified strain of SARS-CoV-2. The primary series of vaccine may be an mRNA vaccine encoding an antigen having a spike protein such as the antigen having an amino acid sequence of SEQ ID NO: 36 or domain or subunit thereof. A subsequent booster or booster series of vaccines is then administered, for instance, shortly after the original vaccine or at a significantly later time in the vaccination protocol (e.g., after neutralizing Ab titers have dropped or after approval of a new strain vaccine).

In aspects disclosed herein, emerging SARS-CoV-2 variant strains are used to design mRNA “boost” as a supplement to prior administered SARS-CoV-2 vaccines and includes traditional boosts, seasonal boosts and pandemic shift boosts. A boost, as used herein refers to any subsequent dose. A traditional boost is a second administration of an antigen administered to a subject following a period of time, such as 21-28 days or even 2 weeks to 6 months. The traditional boost involves the administration of the same antigen representing the same virus strain to the subject in order to generate a robust immune response against that viral strain and optionally other variant strains.

During a pandemic or endemic, emerging viral strains may develop which are not effectively susceptible to neutralization with a vaccine designed against the original strain. In particular, SARS-CoV-2 emerging viral strains appear to arise through radial evolution; that is, with a variety of different mutations, as compared to linear evolution, in which mutations accumulate upon one another as the virus evolves. In such instances, a pandemic shift boost may be used to provide immune protection against emerging viral strains. A pandemic shift boost is a subsequent vaccine which is administered to a subject following a complete course of a first vaccine. The complete course of the first vaccine may comprise one or more administrations of the first vaccine. The pandemic shift boost is comprised of a vaccine that includes an antigen which is derived from a variant viral strain that has emerged during a pandemic or endemic of the viral infection. The pandemic shift boost may be administered at any time following the administration of the first vaccine. The first vaccine may be a vaccine against the originally detected strain of the virus, a combination of the original strain of the virus and variant strain(s) of the virus, or variant strains of the virus, as long as the pandemic shift boost comprises a vaccine against a different variant strain of the virus from the first vaccine.

Additionally variant viral strains of SARS-CoV-2 may emerge at times outside of a pandemic or endemic. These strains may emerge, for instance, seasonally. Such variant strains may be used to design seasonal SARS-CoV2 vaccines which as delivered as a seasonal boost. A seasonal boost is a subsequent dose which is administered to a subject following a complete course of a first vaccine which happens outside of a pandemic or endemic, as variant strains arise. Viral surveillance methods are used in the design of traditional vaccines. However, due to the slow development time of traditional vaccines, the antigen design decisions are often made so far in advance that the vaccine does not match the viral strains circulating when the vaccines are administered. During the development period the viruses may mutate, or other strains may become more prevalent, such that the traditional vaccines become less effective. The traditional vaccines cannot adapt because they are already in production, and it would take additional time to design and manufacture a new vaccine. In contrast, the mRNA vaccines described herein are able to overcome these challenges. They can be produced in a matter of weeks, so that they can be designed against the coronaviruses circulating closer to the inoculation date. For instance, a seasonal or annual coronavirus vaccination program can be developed that rapidly develops a coronavirus vaccine in response to viral strains circulating at the time of vaccination. That is, it is thought that prediction of the viruses closer to a coronavirus season or other outbreak will be more accurate than predictions from several months before the season or the outbreak begins, and therefore the mRNA vaccines described herein will also be more effective because they are designed to target circulating viruses closer to the coronavirus season or scheduled inoculations. Thus, in exemplary aspects, the vaccines of the disclosure may be designed to combat seasonal coronavirus strains, and as such are vaccines for use in an upcoming or forthcoming Northern hemisphere season or Southern hemisphere season. Based on an understanding of circulating coronaviruses at a given point in time, the vaccines are designed to combat such viruses as they are predicted to be those that will be circulating or prevalent in the upcoming or forthcoming virus season. The mRNA vaccines can be designed in a matter of days and a recent vaccine developed by applicant preceded from design to manufactured vaccine in just over 5 weeks. Data can be captured and analyzed as to what viruses are circulating and with what prevalence, much closer to the start of an inoculation program such as seasonal vaccination.

A key protein on the surface of coronavirus, including the SARS-CoV-2 and mutant strains described herein, is the Spike (S) protein such as the spike protein having an amino acid sequence of SEQ ID NO: 36. It has been found that there are particular regions of the Spike protein that relate to the virus' ability to infect cells in vivo. Therefore, in some embodiments, the vaccines used herein are mRNA encoding a spike protein domain, such as a receptor binding domain (RBD), an N-terminal domain (NTD), or a combination of an RBD and NTD. Both the RBD and the NTD have been found to be related to the ability of the vaccine to neutralize the virus. In some embodiments the domain antigens such as RBD comprise a 2P stabilizing mutation. The vaccination protocols described herein also comprise various doses and dosages of mRNA encoding domains, subunits and full length proteins from the original SARS-CoV-2 strain and/or emerging variant SARS-CoV-2 strains. In some embodiments the antigens disclosed herein do not include a 2P stabilized spike protein.

In some embodiments, the vaccine comprises a nucleic acid encoding a Spike protein antigen having at least one RBD mutation as compared to the USA-WA1/2020 isolate reference strain. In some embodiments, the vaccine comprises a nucleic acid encoding a Spike protein antigen comprises at least one RBD mutation selected from the group consisting of K417N, E484K, and N50Y as compared to the USA-WA1/2020 isolate reference strain. In some embodiments, the vaccine comprises a nucleic acid encoding a Spike protein antigen comprising two or all three RBD mutation selected from the group consisting of K417N, E484K, and N50Y as compared to the USA-WA1/2020 isolate reference strain.

A variety of mRNA constructs have been designed and are disclosed herein. When formulated in appropriate delivery vehicles mRNA encoding a spike antigen, domains and/or subunits thereof of emerging variant strains are capable of inducing a strong immune response against SARS-CoV-2, thus producing effective and potent mRNA vaccines/boosters to provide the diversity essential to eradicating the original virus as well as subsequent strains. Intramuscular administration of the mRNA encoding various antigens in an LNP results in delivery of the mRNA to immune tissues and cells of the immune system where it is rapidly translated into proteins antigens. Other immune cells, for example, B cells and T cells, are then able to recognize and mount an immune response against the encoded protein and ultimately create a long-lasting protective response against the coronavirus. Low immunogenicity, a drawback in protein vaccine development due to poor presentation to the immune system or incorrect folding of the antigens, is avoided through the use of the highly effective mRNA vaccines encoding spike protein, subunits and domains thereof disclosed herein.

Due to the constant evolving nature of viruses, scientists continuously monitor the sequences and strains of viruses circulating in humans. These various circulating strains may be used as boost or individual vaccines as disclosed herein, or additionally to design multivalent mRNA vaccines. Viral surveillance can be used to provide annual or seasonal (or other scheduled) information to select the precise virus strains to be used as the basis of mRNA vaccines. Once circulating strains are identified, the composition of a vaccine that targets two or three (or more) most representative virus types in circulation can be developed based on those strains. This exercise of adding antigens from new strains to the vaccine can be repeated on an annual basis or other time frame as required to maintain viral immunity in the population. The mRNA vaccines described herein, in some embodiments, encode multiple antigens from multiple circulating strains in a single lipid nanoparticle (LNP). The mRNA vaccines comprise, in some embodiments, a combination of at least two antigens, each derived from a unique strain of coronavirus.

Thus, the present disclosure provides compositions (e.g., mRNA vaccines) that elicit potent neutralizing antibodies against coronavirus antigens in a subject. Such a composition can be administered to seropositive or seronegative subjects in some embodiments. A seronegative subject may be naïve and not have antibodies that react with SARS-CoV-2. A seropositive subject may have preexisting antibodies to SARS-CoV-2 because they have previously had an infection with SARS-CoV-2 or may have previously been administered a dose of a vaccine (e.g., an mRNA vaccine) that induces antibodies against SARS-CoV-2. In some embodiments, a composition includes mRNA encoding at least one (e.g., one, two, or more) coronavirus antigens, such as SARS-CoV-2 antigens from different SARS-CoV-2 mutant strains (also referred to herein as variants). In some embodiments, the mRNA vaccine comprises multiple mRNAs encoding SARS-CoV-2 antigens from different variants in a single lipid nanoparticle. In some embodiments, the mRNA vaccine comprises an mRNA encoding a SARS-CoV-2 antigen comprising one or more mutations from at least two different SARS-CoV-2 variants (e.g., encoding a combination of the mutations and/or deletions found in the B.1.1.7 and 5021.V2 variants). Table 2, below, presents examples of Spike protein mutations in SARS-CoV-2 variants.

TABLE 2
Spike mutations in SARS-CoV-2 variants

| Variant Name | Amino Acid Changes in Spike |
|---|---|
| 20A.EU1 | A222V-D614G |
| 20A.EU2 | S477N-D614G |
| N439K-D614G | N439K-D614G |
| Mink Cluster 5 Variant | ΔH69ΔV70-Y453F-D614G-I692V-M1229I |
| B.1.1.7 | ΔH69ΔV70-ΔY144-N501Y-A570D- |
| (a.k.a., 20I/501Y.V1, | D614G-P681H-T716I-S982A-D1118H |
| VOC 202012/01) | |
| B.1.1.7-E484K | ΔH69ΔV70-ΔY144-E484K-N501Y- |
| | A570D-D614G-P681H-T716I- |
| | S982A-D1118H |
| B.1.351 | L18F-D80A-D215G-ΔL242ΔA243ΔL244- |
| (a.k.a., 20H/501Y.V2) | R246I-K417N-E484K-N501Y- |
| | D614G-A701V |
| P.1 | L18F-T20N-P26S-D138Y-R190S- |
| | K417T-E484K-N501Y-D614G- |
| | H655Y-T1027I |
| B.1.429 | S13I, W152C, L452R, D614G |
| (a.k.a., CAL.20C) | |
| B.1.617.1 v1 (a.k.a., | T95I-G142D-E154K-L452R-E484Q- |
| India Variant v1) | D614G-P681R-Q1071H |
| B.1.617.1 v2 (a.k.a., | G142D-E154K-L452R-E484Q-D614G- |
| India Variant v2) | P681R-Q1071H-H1101D |
| A.VOI.V2 (a.k.a. | D80Y-ΔY144-ΔI210-D215G-ΔR246- |
| Angola Variant) | ΔS247-ΔY248-L249M-W258L-R346K- |
| | T478R-E484K-H655Y-P681H-Q957H |
| B.1.617.2 (Delta | T19R-G142D-E156G-F157del-R158del- |
| variant; Indian) | L452R-T478K-D614G-P681R-D950N |
| B.1.1.529 | A67V, Δ69-70, T95I, G142D/Δ143-145, |
| (Omicron variant; | Δ211/L212I, ins214EPE, G339D, S371L, |
| Botswana) | S373P, S375F, K417N, N440K, G446S, |
| | S477N, T478K, E484A, Q493K, G496S, |
| | Q498R, N501Y, Y505H, T547K, D614G, |
| | H655Y, N679K, P681H, N764K, D796Y, |
| | N856K, Q954H, N969K, L981F |

At least four groups of SARS-CoV-2 mutants are currently of concern due to increasing prevalence, higher hACE2 binding affinity, or reported escape from mAb and convalescent sera. One exemplary circulating strain (UK) of coronavirus is N501Y-UK, or B.1.1.7 (alpha variant), which has the following mutations: ΔH69-ΔV70-ΔY144-N501Y-A570D-P681H-T716I-S982A-D1118H. This strain has been observed to spread quickly through a region. N501Y causes increased binding affinity to hACE2, making viral uptake more likely. ΔH69-ΔV70 has been shown to have reduced sensitivity to convalescent sera and P681H locates immediately adjacent to furin cleavage site.

Another strain (South Africa), N501Y-SA (B.1.351) (beta variant) with a K417N-E484K-N501Y mutation has also shown fast regional spread and higher viral load in patients. E484K has been shown to have reduced sensitivity to convalescent sera. Both N501Y and E484K are located in the receptor binding domain (RBD) and these mutations increase RBD binding affinity to hACE2.

An additional strain, identified in Japan from four people traveling from Brazil, P.1 (B1.1.248; 20I/501Y.V1) (gamma variant) has emerged. This variant contains 12 mutations in its spike protein, including N501U and E484K. It is thought that the further mutations that may affect its ability to be recognized by antibodies and it is thought to be more transmissible than the wild-type virus (USA-WA1/2020 isolate).

The B.1.429 (also called CAL.20C or 542R.V1) strain (epsilon variant) was found at Cedars-Sinai Medical Center in Los Angeles. The variant contains five mutations: I4205V (ORF1a), D1183Y (ORF1b), and S13I, W152C, and L452R (spike protein) (Zhang et al., medRxiv preprint, Jan. 20, 2021). The L452R mutation is located within the RBD and has been found to be resistant to certain monoclonal antibodies against the spike protein.

An additional subclade of B.1.1.248 has emerged in Amazonas state, Brazil to cause concern over re-infection of people previously infected. The two subclades of B.1.1.28 are designated P.2 (alias of B.1.1.28.2) and P.1 (alias of B.1.1.28.1). To be clear, the variant of concern (VOC) is the subclade designated P.1 (alias of B.1.1.28.1) that has caused a noted re-infection of a woman previously infected and who previously had recovered. The reinfection may be the result of limited or transitory immunity induced in the initial infection or it may reflect a superior ability of the new strain to evade previous immune responses. This new strain contains 12 spike protein mutations including 3 in the RBD (K417T, E484K, N501Y) and one new N-glycosylation site at T20N. The S protein mutations include the following 12 mutations: L18F, T20N, P26S, D138Y, R190S, K417T, E484K, N501Y, D614G, H655Y, T1027I, V1176F (see Naveca et al., SARS-CoV-2 reinfection by the new Variant of Concern (VOC) P.1 in Amazonas, Brazil, 2021). The reinfection caused similar moderate symptoms as the initial infection, but recorded a higher viral load in nasopharyngal and pharyngeal samples. The reinfection may be the result of the E484K mutation in the spike protein and its ability to facilitate evasion of SARS-CoV-2 neutralizing antibodies.

In Germany, a new variant has been detected in 35 out of 73 new patients in Garmisch-Partenkirchen. The variant is currently being sequenced, although at least one point mutation has been detected in the spike protein.

In India, two related variants, both belonging to the B.1.617.1 subclade, have emerged. The genome of one B.1.617.1 variant, referred to as v1, or B.1.617.1 v1 (kappa variant), encodes a Spike protein having the following 8 substitutions: T95I, G142D, E154K, L452R, E484Q, D614G, P681R, and Q1071H. The genome of the other B.1.617.1 variant, referred to as v2, or B.1.617.1 v2, encodes a Spike protein with the following 8 substitutions: G142D, E154K, L452R, E484Q, D614G, P681R, Q1071H, and H1101D.

Also in India, another variant, belonging to the B.1.617.2 subclade, has emerged. The genome of the B.1.617.2 (Delta) variant encodes a Spike protein having the following ten substitutions: T19R, G142D, E156G, F157, R158, L452R, T478K, D614G, P681R, D950N, in addition to two deletions: F157del and R158del.

In Angola, a new variant, referred to as A.VOI.V2, with multiple Spike protein mutations has been detected through genomic surveillance. The genome of the A.VOI.V2 variant encodes a Spike protein having the following 15 mutations, including 10 substitutions and 5 amino acid deletions: D80Y, ΔY144, ΔI210, D215G, ΔR246, ΔS247, ΔY248, L249M, W258L, R346K, T478R, E484K, H655Y, P681H, and Q957H.

A variant of interest (VOI), lambda (C.37) has been investigated. The variant was first documented in Peru and is most frequently found in South America. It has relatively high risk scores, due mainly to its high number of deletions in the N-terminal domain (NTD) and it is possible that its RSYLTPGD246-253N mutation may increase its ability to evade neutralizing antibodies.

Another variant of interest (VOI), mu (B.1621) has been reported and first documented in Colombia. The variant comprises an insertion, 146N, and several amino acid substitutions in the Spike protein (Y144T, Y145S, R346K, E484K, N501Y and P681H).

Two additional related variants, B.1.243 and B.1.243.1 have been found primarily in North America (Arizona). The B.1.243.1 variant has the E484K mutation, in addition to a further Spike protein mutation (V213G), which may render it more resistant to neutralizing antibodies.

A variant of concern, Omicron (B.1.1.529), having multiple Spike protein mutations was detected initially in Botswana. The mutations observed in the variant include those found in the Delta variant that are believed to increase transmissibility and mutations, and those seen in the Beta and Delta variants that are believed to promote immune escape. In particular, the genome of the Omicron variant encodes a Spike protein having the following mutations: A67V, A69-70, T95I, G142D/Δ143-145, Δ211/L212I, ins214EPE, G339D, S371L, S373P, S375F, K417N, N440K, G446S, S477N, T478K, E484A, Q493K, G496S, Q498R, N501Y, Y505H, T547K, D614G, H655Y, N679K, P681H, N764K, D796Y, N856K, Q954H, N969K, and L981F.

These exemplary strains and other newly emerging strains (e.g., additional variants of concern) are candidates for the methods and formulations disclosed herein. mRNA encoding antigens from these and other coronavirus strains have been designed for mRNA vaccines. In some embodiments these variants comprise at least one amino acid substitution in the S1 or S2 domains of a coronavirus strain relative to a parent strain. In some embodiments these variants comprise one or more amino acid deletions in the S1 or S2 domains of a coronavirus strain relative to a parent strain. A parent strain, as used herein, is a reference viral strain. A variant strain has at least 1 structural difference (at least one amino acid substituted, deleted, modified or added) and at least one functional difference (relative to type of function, degree or intensity of function) relative to a parent strain. The terminology parent and variant do not imply that either was in existence prior to one another but rather, serves as a reference for structure/function. Function may imply, for instance, increased transmissibility, increased morbidity, increased mortality, increased risk of “long COVID” (e.g., post-acute sequelae of SARS-CoV-2 infection), ability to evade detection by diagnostic tests, decreased susceptibility to antiviral drugs, decreased susceptibility to neutralizing antibodies, ability to evade natural immunity, ability to infect vaccinated individuals, increased risk of particular conditions (e.g., multisystem inflammatory syndrome), and increased affinity for a particular demographic or clinical groups (e.g., children, immunocompromised individuals), relative to the original strain or other variant of the virus

In some embodiments, the mRNA vaccines described herein may be administered as a prime or priming immunization (e.g., the first administration of a coronavirus vaccine to a subject). In some embodiments, the mRNA vaccines described herein may be administered as a booster, that is, a dose administered after the prime or priming immunization, as described herein. In some embodiments, the booster and the prime or priming immunization comprise the same mRNA or mRNAs. In other embodiments, the booster and the prime or priming immunization comprise different mRNA or mRNAs. In other embodiments multiple mRNA vaccines encoding different antigens (each directed at a strain or multiple strains) may be administered together or in tandem to provide a wide spectrum neutralization platform against multiple coronavirus strains.

### SARS-CoV-2

The present disclosure provides compositions (e.g., mRNA vaccines) and vaccination methods that elicit potent neutralizing antibodies against coronavirus antigens. The genome of SARS-CoV-2 is a single-stranded positive-sense RNA (+ssRNA) with the size of 29.8-30 kb encoding about 9860 amino acids (Chan et al. 2000, supra; Kim et al. 2020 Cell, May 14; 181(4):914-921.e10.). SARS-CoV-2 is a polycistronic mRNA with 5′-cap and 3′-poly-A tail. The SARS-CoV-2 genome is organized into specific genes encoding structural proteins and nonstructural proteins (Nsps). The order of the structural proteins in the genome is 5′-replicase (open reading frame (ORF)1/ab)-structural proteins [Spike (S)-Envelope (E)-Membrane (M)-Nucleocapsid (N)]-3′. The genome of coronaviruses includes a variable number of open reading frames that encode accessory proteins, nonstructural proteins, and structural proteins (Song et al. 2019 Viruses; 11(1):p. 59). Most of the antigenic peptides are located in the structural proteins (Cui et al. 2019 Nat. Rev. Microbiol.; 17(3):181-192). Spike surface glycoprotein (S), a small envelope protein (E), matrix protein (M), and nucleocapsid protein (N) are four main structural proteins. Since S-protein contributes to cell tropism and virus entry and also it is capable to induce neutralizing antibodies (NAb) and protective immunity, it can be considered one of the most important targets in coronavirus vaccine development among all other structural proteins. Moreover, amino acid sequence analysis has shown that S-protein contains conserved regions among the coronaviruses, which may be the basis for universal vaccine development.

### Antigens

The compositions of the invention, e.g., vaccine compositions, feature nucleic acids, in particular, mRNAs, designed to encode an antigen of interest, e.g., an antigen derived from a betacoronavirus structural protein, in particular, antigens derived from SARS-CoV-2 Spike protein. The compositions of the invention, e.g., vaccine compositions, do not comprise antigens per se, but rather comprise nucleic acids, in particular, mRNA(s) that encode antigens or antigenic sequences once delivered to a cell, tissue or subject. Delivery of nucleic acids, in particular mRNA(s) is achieved by formulating said nucleic acids in appropriate carriers or delivery vehicles (e.g., lipid nanoparticles) such that upon administration to cells, tissues or subjects, nucleic acid is taken up by cells which, in turn, express protein(s) encoded by the nucleic acids, e.g., mRNAs.

Antigens, as used herein, are proteins capable of inducing an immune response (e.g., causing an immune system to produce antibodies against the antigens). The vaccines of the present disclosure provide a unique advantage over traditional protein-based vaccination approaches in which protein antigens are purified or produced in vitro, e.g., recombinant protein production technologies. The vaccines of the present disclosure feature mRNA encoding the desired antigens, which when introduced into the body, i.e., administered to a mammalian subject (for example a human) in vivo, cause the cells of the body to express the desired antigens. In order to facilitate delivery of the mRNAs of the present disclosure to the cells of the body, the mRNAs are encapsulated in lipid nanoparticles (LNPs). Upon delivery and uptake by cells of the body, the mRNAs are translated in the cytosol and protein antigens are generated by the host cell machinery. The protein antigens are presented and elicit an adaptive humoral and cellular immune response. Neutralizing antibodies are directed against the expressed protein antigens and hence the protein antigens are considered relevant target antigens for vaccine development. Herein, use of the term “antigen” encompasses immunogenic proteins and immunogenic fragments (an immunogenic fragment that induces (or is capable of inducing) an immune response to a (at least one) SARS-CoV-2 variant), unless otherwise stated. It should be understood that the term “protein” encompasses peptides and the term “antigen” encompasses antigenic fragments. Other molecules may be antigenic such as bacterial polysaccharides or combinations of protein and polysaccharide structures, but for the viral vaccines included herein, viral proteins, fragments of viral proteins and designed and or mutated proteins derived from SARS-CoV-2 are the antigens provided herein.

Many proteins have a quaternary or three-dimensional structure, which consists of more than one polypeptide or several polypeptide chains that associate into an oligomeric molecule. As used herein the term “subunit” refers to a single protein molecule, for example, a polypeptide or polypeptide chain resulting from processing of a nascent protein molecule, which subunit assembles (or “coassembles”) with other protein molecules (e.g., subunits or chains) to form a protein complex. Proteins can have a relatively small number of subunits and therefore be described as “oligomeric” or can consist of a large number of subunits and therefore be described as “multimeric”. The subunits of an oligomeric or multimeric protein may be identical, homologous or totally dissimilar and dedicated to disparate tasks.

Proteins or protein subunits can further comprise domains. As used herein, the term “domain” refers to a distinct functional and/or structural unit within a protein. Typically, a “domain” is responsible for a particular function or interaction, contributing to the overall role of a protein. Domains can exist in a variety of biological contexts. Similar domains (i.e., domains sharing structural, functional and/or sequence homology) can exist within a single protein or can exist within distinct proteins having similar or different functions. A protein domain is often a conserved part of a given protein tertiary structure or sequence that can function and exist independently of the rest of the protein or subunit thereof.

In structural and molecular biology, identical, homologous or similar subunits or domains can help to classify newly identified or novel proteins, as was done immediately upon publication of the SARS-CoV-2 viral genomic sequence.

As used herein, the term antigen is distinct from the term “epitope” which is a substructure of an antigen, e.g., a polypeptide, such as 7-10 amino acids, or carbohydrate structure, which may be recognized by an antigen binding site but is insufficient to induce an immune response. The art describes protein antigens that are delivered to subjects or immune cells in isolated form, e.g., isolated protein, polypeptide or peptide antigens, however, the design, testing, validation, and production of protein antigens can be costly and time-consuming, especially when producing proteins at large scale. By contrast, mRNA technology is amenable to rapid design and testing of mRNA constructs encoding a variety of antigens. Moreover, rapid production of mRNA coupled with formulation in appropriate delivery vehicles (e.g., lipid nanoparticles), can proceed quickly and can rapidly produce mRNA vaccines at large scale. Potential benefit also arises from the fact that antigens encoded by the mRNAs of the invention are expressed by the cells of the subject, e.g., are expressed by the human body, and thus the subject, e.g., the human body, serves as the “factory” to produce the antigens which, in turn, elicits the desired immune response.

The compositions, as provided herein, may include an RNA or multiple RNAs encoding two or more antigens of the same or different viral strains. Also provided herein are combination vaccines that include RNA encoding one or more coronavirus antigens and one or more antigen(s) of a different organism. Thus, the vaccines of the present disclosure may be combination vaccines that target one or more antigens of the same strain/species, or one or more antigens of different strains/species, e.g., antigens which induce immunity to organisms which are found in the same geographic areas where the risk of coronavirus infection is high or organisms to which an individual is likely to be exposed to when exposed to a coronavirus (e.g., COVID-19).

### Encoded Coronavirus Spike (S) Protein Antigens

The envelope spike (S) proteins of known betacoronaviruses determine the virus host tropism and entry into host cells. Coronavirus spike (S) protein is a choice antigen for the vaccine design as it can induce neutralizing antibodies and protective immunity. S protein is critical for SARS-CoV-2 infection. The organization of the S protein is similar among betacoronaviruses, such as SARS-CoV-2, SARS-CoV, MERS-CoV, HKU1-CoV, MHV-CoV and NL63-CoV.

As used herein, the term “Spike protein” refers to a glycoprotein that that forms homotrimers protruding from the envelope (viral surface) of viruses including betacoronaviruses. Trimerized Spike protein facilitates entry of the virion into a host cell by binding to a receptor on the surface of a host cell followed by fusion of the viral and host cell membranes. The S protein is a highly glycosylated and large type I transmembrane fusion protein that is made up of 1,160 to 1,400 amino acids, depending upon the type of virus. Betacoronavirus Spike proteins comprise between about 1100 to 1500 amino acids.

SARS-CoV-2 spike (S) protein is a choice antigen for the vaccine design as it can induce neutralizing antibodies and protective immunity. mRNAs of the invention are designed to produce SARS-CoV-2 Spike proteins (i.e., encode Spike proteins such that Spike protein is expressed when the mRNA is delivered to a cell or tissue, for example a cell or tissue in a subject), as well as antigenic variants thereof. The skilled artisan will understand that, while an essentially full length or complete Spike protein may be necessary for a virus, e.g., a betacoronavirus, to perform its intended function of facilitating virus entry into a host cell, a certain amount of variation in Spike protein structure and/or sequence is tolerated when seeking primarily to elicit an immune response against Spike protein. For example, minor truncation, e.g., of one to a few, possibly up to 5 or up to 10 amino acids from the N- or C-terminus of the encoded Spike protein, e.g., encoded Spike protein antigen, may be tolerated without changing the antigenic properties of the protein. Likewise, variation (e.g., conservative substitution) of one to a few, possibly up to 5 or up to 10 amino acids (or more) of the encoded Spike protein, e.g., encoded Spike protein antigen, may be tolerated without changing the antigenic properties of the protein. In some embodiments, the Spike protein is not a stabilized Spike protein, for example, the Spike protein is stabilized by two proline substitutions (a 2P mutation).

In some embodiments, the Spike protein is from a different virus strain. A strain is a genetic variant of a microorganism (e.g., a virus). New viral strains can be created due to mutation or swapping of genetic components when two or more viruses infect the same cell in nature, for example, by antigenic drift or antigenic shift.

Antigenic drift is a kind of genetic variation in viruses, arising by the accumulation of mutations in the virus genes that code for virus-surface proteins that host antibodies recognize. This results in a new strain of virus particles that is not effectively inhibited by the antibodies that prevented infection by previous strains. This makes it easier for the changed virus to spread throughout a partially immune population.

Antigenic shift is the process by which two or more different strains of a virus, or strains of two or more different viruses, combine to form a new subtype having a mixture of the surface antigens of the two or more original strains. The term is often applied specifically to influenza, as that is the best-known example, but the process is also known to occur with other viruses. Antigenic shift is a specific case of reassortment or viral shift that confers a phenotypic change. Antigenic shift is contrasted with antigenic drift, which is the natural mutation over time of known strains of a virus which may lead to a loss of immunity, or in vaccine mismatch. Antigenic shift is often associated with a major reorganization of viral surface antigens resulting in a reassortment change the virus's phenotype drastically.

A virus strain as used herein is a genetic variant or of a virus that is characterized by a differing isoform of one or more surface proteins of the virus. In the case of SARS-CoV-2, for example, a different amino acid sequence in the SARS-CoV-2 spike protein where the immune response in an individual to the new strain is less effective than to the strain used to immunize or first infect the individual. A new virus strain may arise from natural mutation or a combination of natural mutation and immune selection due to an ongoing immune response in an immunized or previously infected individual. A new virus strain can differ by one, two, three or more amino acid mutations in regions of the spike protein responsible for a viral function such as receptor binding or viral fusion with a target cell. A spike protein from a new strain will typically have more than 80%, 85%, 90%, 95%, 96%, 97%, 98%, 99%, 99.3%, 99.5% or 99.8% sequence identity at the amino acid level with a parental strain. A spike protein from a new strain may differ from the parental strain by as much as 80%, 85%, 90%, 95%, 98%, 99% identity at the amino acid level.

A natural virus strain is a variant of a given virus that is recognizable because it possesses some “unique phenotypic characteristics” that remain stable (e.g., stable and heritable biological, serological, and/or molecular characters) under natural conditions. Such “unique phenotypic characteristics” are biological properties different from the compared reference virus, such as unique antigenic properties, host range (e.g., infecting a different kind of host), symptoms of disease caused by the strain, different type of disease caused by the strain (e.g., transmitted by different means), etc. A “unique phenotypic characteristic” can be detected clinically (e.g., clinical manifestations detected in a host infected with the strain) or within a comparative animal experiment in which a researcher skilled in the art of virology can distinguish between the reference control virus-infected animal and the animal infected with the alleged new strain, without knowing which animal received which virus and without having any information about the differences between the two viruses. Importantly, a virus variant with a simple difference in genome sequence is not a separate strain if there is no recognizable distinct viral phenotype. The extent of genomic sequence variation is irrelevant for the classification of a variant as a strain since a distinct phenotype sometimes arises from few mutations.

As an example, in some embodiments, the mRNA encodes an antigen from at least one virus strain variant or comprises mutations from at least one virus strain that is not wild-type SARS-CoV-2. In some embodiments, the vaccine comprises mRNA encoding a Spike protein associated with the B.1.1.7 lineage (UK) variant (20B/501Y.V1 VOC 202012/01). The B.1.1.7 lineage variant has a mutation in the receptor binding domain (RBD) of the Spike protein at position 501, where amino acid asparagine (N) has been replaced with tyrosine (Y); an N501Y mutation. Further, the variant has a 69/70 deletion, which occurs spontaneously numerous times, leading to conformation changes in the Spike protein, a P681H mutation near the S1/S2 furin cleavage site, and a ORF8 stop codon (Q27 stop) caused by a mutation in ORF8. The 501.V2 (South Africa, SA) variant comprises multiple mutations in the Spike protein, including N501Y, and E484K, but does not have a deletion at 69/70. The E484K mutation is considered to be an “escape” mutation relative to at least one form of monoclonal antibody against SARS-CoV-2, such that it may change the antigenicity of the virus. Other mutations that have been discovered include the D614G mutation, which is thought to increase the transmission rate of the virus, and the N543Y mutation (emerged from mink farms in the Netherlands and Denmark). In some embodiments, the Spike protein comprises mutations from more than one variant (e.g., a combination of mutations found in the B.1.1.7 and 502Y.V2 variants).

In exemplary embodiments, a Spike protein, e.g., an encoded Spike protein antigen, has the amino acid sequence set forth in any one of SEQ ID NOs: 20, 23, 26, 29, 32, 57, 60, and 63. In other embodiments, a Spike protein, e.g., an encoded Spike protein antigen, has no greater than 100, no greater than 90, no greater than 80, no greater than 70, no greater than 60, no greater than 50, no greater than 40, no greater than 30, no greater than 20, no greater than 10, or no greater than 5 amino acid substitutions and/or deletions as compared to (when aligned with) a Spike protein having the amino acid sequence as set forth in any one of SEQ ID NOs: 20, 23, 26, 29, 32, 57, 60, and 63. Where minor variations are made in encoded Spike protein sequences, the variant preferably has the same activity as the reference Spike protein sequence and/or has the same immune specificity as the reference Spike protein, as determined for example, in immunoassays (e.g., enzyme-linked immunosorbent assays (ELISA assays).

S proteins of coronaviruses can be divided into two important functional subunits, of which include the N-terminal S1 subunit, which forms of the globular head of the S protein, and the C-terminal S2 region that forms the stalk of the protein and is directly embedded into the viral envelope. Upon interaction with a potential host cell, the S1 subunit will recognize and bind to receptors on the host cell, specifically angiotensin-converting enzyme 2 (ACE2) receptors, whereas the S2 subunit, which is the most conserved component of the S protein, will be responsible for fusing the envelope of the virus with the host cell membrane. (See e.g., Shang et al., PLoS Pathog. 2020 March; 16(3):e1008392.). Each monomer of trimeric S protein trimer contains the two subunits, S1 and S2, mediating attachment and membrane fusion, respectively. As part of the infection process in vivo, the two subunits are separated from each other by an enzymatic cleavage process. S protein is first cleaved by furin-mediated cleavage at the S1/S2 site in infected cells, In vivo, a subsequent serine protease-mediated cleavage event occurs at the S2′ site within S1. In SARS-CoV2, the S1/S2 cleavage site is at amino acids 676-TQTNSPRRAR/SVA-688 (referencing SEQ ID NO: 35). The S2′ cleavage site is at amino acids 811-KPSKR/SFI-818 (SEQ ID NO: 66).

As used herein, for example in the context of designing SARS-CoV-2 S protein antigens encoded by the nucleic acids, e.g., mRNAs, of the invention, the term “S1 subunit” (e.g., S1 subunit antigen) refers to the N-terminal subunit of the Spike protein beginning at the S protein N-terminus and ending at the S1/S2 cleavage site whereas the term “S2 subunit” (e.g., S2 subunit antigen) refers to the C-terminal subunit of the Spike protein beginning at the S1/S2 cleavage site and ending at the C-terminus of the Spike protein. As described supra, the skilled artisan will understand that, while an essentially full length or complete Spike protein S1 or S2 subunit may be necessary for receptor binding or membrane fusion, respectively, a certain amount of variation in S1 or S2 structure and/or sequence is tolerated when seeking primarily to elicit an immune response against Spike protein subunits. For example, minor truncation, e.g., of one to a few, possibly up to 4, 5, 6, 7, 8, 9 or 10 amino acids from the N- or C-terminus of the encoded subunit, e.g., encoded S1 or S2 protein antigens, may be tolerated without changing the antigenic properties of the protein. Likewise, variation (e.g., conservative substitution) of one to a few, possibly up to 4, 5, 6, 7, 8, 9 or 10 amino acids (or more) of the encoded Spike protein subunits, e.g., encoded S1 or S2 protein antigen, may be tolerated without changing the antigenic properties of the protein(s). In exemplary embodiments, a Spike protein, e.g., an encoded Spike protein antigen, has the amino acid sequence set forth in any one of SEQ ID NOs: 20, 23, 26, 29, or 32.

The S1 and S2 subunits of the SARS-CoV-2 Spike protein further include domains readily discernable by structure and function, which in turn can be featured in designing antigens to be encoded by the nucleic acid vaccines, in particular, mRNA vaccines of the invention. Within the S1 subunit, domains include the N-terminal domain (NTD) and the receptor-binding domain (RBD), said RBD domain further including a receptor-binding motif (RBM) Within the S2 subunit, domains include fusion peptide (FP), heptad repeat 1 (HR1), heptad repeat 2 (HR2), transmembrane domain (TM), and cytoplasm domain, also known as cytoplasmic tail (CT) (Lu R. et al., supra; Wan et al., J. Virol. March 2020, 94 (7) e00127-20). The HR1 and HR2 domains can be referred to as the “fusion core region” of SARS-CoV-2 (Xia et al., 2020 Cell Mol Immunol. January; 17(1):1-12.). The S1 subunit includes an N terminal domain (NTD), a linker region, a receptor binding domain (RBD), a first subdomain (SD1), and a second subdomain (SD2). The S2 subunit includes, inter alia, a first heptad repeat (HR1), a second heptad repeat (HR2), a transmembrane domain (TM), and a cytoplasmic tail. The NTD and RBD of S1 are good antigens for the vaccine design approach of the invention as these domains have been shown to be the targets of neutralizing antibodies in betacoronavirus-infected individuals. As used herein, for example, in the context of an antigen design (said antigen encoded by an mRNA of the invention and to be expressed, for example, from and mRNA vaccine of the invention), the term “N-terminal domain” or “NTD” refers to a domain within the SARS-CoV-2 S1 subunit comprising approximately 290 amino acids in length, having identity to amino acids 1-290 of the S1 subunit of the Spike protein having the amino acid sequence set forth as SEQ ID NO: 36. As used herein, for example, in the context of an antigen design (said antigen encoded by an mRNA of the invention and to be expressed, for example, from and mRNA vaccine of the invention), the term “receptor binding domain” or “RBD” refers to a domain within the S1 subunit of SARS-CoV-2 comprising approximately 175-225 amino acids in length, having identity to amino acids 316-517 of the S1 subunit of the Spike protein having the amino acid sequence set forth as SEQ ID NO: 36. As used herein, the term “receptor binding motif” refers to the portion of the RBD that directly contacts the ACE2 receptor. Expressed RBDs are predicted to specifically bind to angiotensin-converting enzyme 2 (ACE2) as its receptor and/or specifically react with RBD-binding and/or neutralizing antibodies, e.g., CR3022. In some embodiments these antigens include a stabilizing 2P mutation.

The compositions provided herein include mRNA that may encode any one or more full-length or partial (truncated or other deletion of sequence) S protein subunit (e.g., S1 or S2 subunit), one or more domain or combination of domains of an S protein subunit (e.g., NTD, RBD, or NTD-RBD fusions, with or without an SD1 and/or SD2), or chimeras of full-length or partial and S2 protein subunits. Other S protein subunit and/or domain configurations are contemplated herein.

In some embodiments, the mRNA vaccine encodes an antigen having at least one of the following mutations relative to wild-type SARS-CoV-2 S protein (SEQ ID NO: 36): E484K, D614G, K417N, N501Y, L18F, D80A, D215G, R246I, A701V, A570D, P861H, T716I, S982A, D1118H. In some embodiments, the mRNA encodes an antigen having 2, 3, 4, 5, 6, 7, 8, 9, 10, 11, 12, 13, or all 14 of the mutations listed. In some embodiments, the mRNA encodes an antigen that has one or more deletions relative to the wild-type SARS-CoV-2 S protein sequence (SEQ ID NO: 36). Exemplary deletions include, but are not limited to, positions, 69, 70, 144, and 242-244. In some embodiments, the mRNA encodes an antigen having 1, 2, 3, 4, 5, or 6 deletions. In some embodiments, the mRNA encoding an antigen has 1, 2, 3, 4, 5, or 6 deletions, and 1, 2, 3, 4, 5, 6, 7, 8, 9, 10, 11, 12, 13, or 14 mutations or any combination thereof.

In some embodiments, the mRNA vaccine comprises 1, 2, 3, 4, 5, or 6 mRNAs encoding different antigens, wherein each antigen comprises at least one mutation and/or at least one deletion. In some embodiments, the mRNA vaccine further comprises an mRNA encoding a wild-type SARS-CoV-2 S protein antigen or the antigenic fragment thereof. The mRNA vaccine, in some embodiments, is in a lipid nanoparticle (that is, the lipid nanoparticle comprises 1, 2, 3, 4, 5, or 6 mRNAs encoding different antigens).

In some aspects, compositions of the disclosure comprise at least: a first mRNA encoding a first SARS-CoV-2 antigen of a first SARS-CoV-2 virus and a second mRNA encoding a second SARS-CoV-2 antigen of a second SARS-CoV-2 virus. In some embodiments, the second SARS-CoV-2 antigen has an amino acid sequence with at least one amino acid mutation, deletion, or both amino acid mutation and deletion with respect to an amino acid sequence of the first SARS-CoV-2 antigen, wherein each of the first and second antigens are not full length stabilized spike proteins having a 2P mutation. In some embodiments, the first SARS-CoV-2 virus is a first circulating SARS-CoV-2 virus. In some embodiments, the second SARS-CoV-2 virus is a second circulating SARS-CoV-2 virus. “Circulating viruses”, as used herein, refers to viruses that have been in circulation, for example, spreading in a population, for 1 month, 2 months, 3 months, 4 months, 5 months, 6 months, 7 months, 8 months, 9 months, 10 months, 11 months, a portion of a year, 1 year, 1.5 years, 2 years, 3 years, or longer. As used herein, “population” refers to a group of subjects. In some embodiments, the population is the worldwide population. In some embodiments, the population is geographically limited (e.g., an African population, a European population) or regionally limited (e.g., Southern hemisphere population).

In some embodiments, the composition further comprises a third mRNA encoding a third SARS-CoV-2 antigen of a third SARS-CoV-2 virus, wherein the third SARS-CoV-2 antigen has an amino acid sequence with at least one amino acid mutation with respect to an amino acid sequence of the first and/or second SARS-CoV-2 antigens.

In some embodiments, the composition further comprises a fourth mRNA encoding a fourth SARS-CoV-2 antigen of a fourth SARS-CoV-2 virus, wherein the fourth SARS-CoV-2 antigen has an amino acid sequence with at least one amino acid mutation with respect to an amino acid sequence of the first, second, and/or third SARS-CoV-2 antigens.

In some embodiments, the composition further comprises a fifth mRNA encoding a fifth SARS-CoV-2 antigen of a fifth SARS-CoV-2 virus, wherein the fifth SARS-CoV-2 antigen has an amino acid sequence with at least one amino acid mutation with respect to an amino acid sequence of the first, second, third and/or fourth SARS-CoV-2 antigens.

In some embodiments, the composition further comprises a sixth mRNA encoding a sixth SARS-CoV-2 antigen of a sixth SARS-CoV-2 virus, wherein the sixth SARS-CoV-2 antigen has an amino acid sequence with at least one amino acid mutation with respect to an amino acid sequence of the first, second, third, fourth and/or fifth SARS-CoV-2 antigens.

In some embodiments, the first antigen and second antigen, and optionally the third, fourth, fifth and sixth antigen are each antigens that share at least 90%, at least 91%, at least 92%, at least 93%, at least 94%, at least 95%, at least 96%, at least 97, at least 98%, or at least 99% sequence identity with one another.

In some embodiments, the first and second antigens are antigens of the spike protein. In some embodiments, the third, fourth, fifth, and/or sixth antigens are antigens of the spike protein.

In some embodiments, the mRNAs are present in the composition in an equal amount (e.g., a 1:1 weight/weight ratio or a 1:1 molar ratio), for example, a ratio of 1:1 (:1:1:1:1) of mRNA encoding distinct coronavirus antigens. As used herein, a “weight/weight ratio” or wt/wt ratio or wt:wt ratio refers to the ratio between the weights (masses) of the different components. A “molar ratio” refers to the ratio between different components (e.g., the number of mRNA encoding each antigen). In some embodiments, the ratio is 1:1, 1:2, 1:3, 1:4, 2:1, 3:1, or 4:1 (first mRNA:second mRNA). In each embodiment or aspect of the invention, it is understood that the featured vaccines include the mRNAs encapsulated within LNPs. While it is possible to encapsulate each unique mRNA in its own LNP, the mRNA vaccine technology enjoys the significant technological advantage of being able to encapsulate several mRNAs in a single LNP product. In other embodiments the vaccines are separate vaccines that are not co-formulated, but may be admixed separately before administration or simply administered separately.

The mutated SARS-CoV-2 antigen encoded by the second mRNA (and/or third, fourth, fifth, or six mRNA), in some embodiments, is associated with a function of the virus that is different in quantity or kind from a corresponding function in the first virus. Exemplary functions include, but are not limited to, ACE2 receptor binding, virus transmissibility, viral uptake, viral pathogenesis, altered furin cleavage, or viral replication. These functions relate to the pathogenicity of the virus.

Exemplary sequences of the coronavirus antigens and the RNA encoding the coronavirus antigens of the compositions of the present disclosure (e.g., SARS-CoV-2 variant antigens) are provided in Table 1. In some embodiments, the mRNA vaccines comprise a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 18, 21, 24, 27, 30, 55, 58, 61, and 64. In some embodiments, the mRNA vaccines encode a polypeptide that is 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 20, 23, 26, 29, 32, 57, 60, and 63.

In some embodiments, the second or subsequent circulating SARS-CoV-2 virus is an immunodominant antigen from an emerging strain. An immunodominant antigen of an emerging strain is assessed with respect to the strain from which the antigen is derived, relative to a different strain of the virus, such as the original strain or other variant thereof. An immunodominant antigen of the emerging strain induces a stronger immune response against the emerging strain than against the different strain.

In some embodiments, the second or subsequent circulating SARS-CoV-2 virus is an immunodominant antigen from a variant of concern (VOC). An immunodominant antigen of VOC is assessed with respect to the strain from which the antigen is derived, relative to a different strain of the virus, such as the original strain or other variant thereof. In some embodiments, the VOC may exhibit increased transmissibility, increased morbidity, increased mortality, increased risk of “long COVID” (e.g., post-acute sequelae of SARS-CoV-2 infection), ability to evade detection by diagnostic tests, decreased susceptibility to antiviral drugs, decreased susceptibility to neutralizing antibodies, ability to evade natural immunity, ability to infect vaccinated individuals, increased risk of particular conditions (e.g., multisystem inflammatory syndrome), and increased affinity for a particular demographic or clinical groups (e.g., children, immunocompromised individuals), relative to the original strain or other variant of the virus.

### Nucleic Acids

The compositions of the present disclosure comprise a (at least one) messenger RNA (mRNA) having an open reading frame (ORF) encoding a coronavirus antigen. In some embodiments, the mRNA further comprises a 5′ UTR, 3′ UTR, a poly(A) tail and/or a 5′ cap analog.

It should also be understood that the coronavirus vaccine of the present disclosure may include any 5′ untranslated region (UTR) and/or any 3′ UTR. Exemplary UTR sequences include SEQ ID NOs: 2, 4, 33, and 34; however, other UTR sequences may be used or exchanged for any of the UTR sequences described herein. In some embodiments, a 5′ UTR of the present disclosure comprises a sequence selected from SEQ ID NO: 33 (GGGAAAUA AGAGAGAAAAGAAGAGUAAGAAGAAAUAUAAGAGCCACC) and SEQ ID NO: 2 (GGGAAAUAAGAGAGAAAAGAAGAGUAAGAAGAAAUAUAAGACCCCGGCGCCGC CACC). In some embodiments, a 3′ UTR of the present disclosure comprises a sequence selected from SEQ ID NO: 34 (UGAUAAUAGGCUGGAGCCUCGGUGGCCAUGCUU CUUGCCCCUUGGGCCUCCCCCCAGCCCCUCCUCCCCUUCCUGCACCCGUACCCCCG UGGUCUUUGAAUAAAGUCUGAGUGGGCGGC) and SEQ ID NO: 4 (UGAUAA UAGGCUGGAGCCUCGGUGGCCUAGCUUCUUGCCCCUUGGGCCUCCCCCCAGCCCC UCCUCCCCUUCCUGCACCCGUACCCCCGUGGUCUUUGAAUAAAGUCUGAGUGGGC GGC). UTRs may also be omitted from the RNA polynucleotides provided herein.

Nucleic acids comprise a polymer of nucleotides (nucleotide monomers). Thus, nucleic acids are also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents mRNA, the “T”s would be substituted for “U”s. Thus, any of the DNAs disclosed and identified by a particular sequence identification number herein also disclose the corresponding mRNA sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an RNA polynucleotide of the present disclosure.

### Variants

In some embodiments, the compositions of the present disclosure include RNA that encodes a SARS-CoV-2 antigen variant. Antigen variants or other polypeptide variants refers to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Examples of SARS-CoV-2 antigen variants are provided in Table 1. Ordinarily, variants possess at least 50% identity to a wild-type, native or reference sequence. In some embodiments, variants share at least 80%, or at least 90% identity with a wild-type, native, or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, a composition comprises an RNA or an RNA ORF that comprises a nucleotide sequence of any one of the sequences provided herein, or comprises a nucleotide sequence at least 90%, at least 95%, at least 96%, at least 97%, at least 98%, or at least 99% identical to a nucleotide sequence of any one of the sequences provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an mRNA vaccine.

As recognized by those skilled in the art, protein fragments, functional protein domains, and homologous proteins are also considered to be within the scope of coronavirus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the coronavirus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition includes an RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a composition includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an mRNA includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, an mRNA includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, an mRNA does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

An mRNA may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an mRNA has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, a composition comprises an mRNA having an ORF that encodes a signal peptide fused to the coronavirus antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than coronavirus antigens in nature) are known in the art and can be tested for desired properties and then incorporated into a nucleic acid of the disclosure.

### Fusion Proteins

In some embodiments, a composition of the present disclosure includes an mRNA encoding an antigenic fusion protein. Thus, the encoded antigen or antigens may include two or more proteins (e.g., protein and/or protein fragment) joined together with or without a linker. Alternatively, the protein to which a protein antigen is fused does not promote a strong immune response to itself, but rather to the coronavirus antigen. Antigenic fusion proteins, in some embodiments, retain the functional property from each original protein.

In some embodiments, a fusion protein comprises a receptor binding domain (RBD) from a SARS-CoV-2 Spike protein.

In some embodiments, a fusion protein comprises an N-terminal domain (NTD) from a SARS-CoV-2 Spike protein

In some embodiments, a fusion protein comprises a transmembrane domain. The transmembrane domain may, in some embodiments, be from a virus that is not SARS-CoV-2. For example, the transmembrane domain may be from an influenza hemagglutinin transmembrane domain, which has been demonstrated to effectively anchor proteins at the cell surface.

### Scaffold Moieties

The mRNA vaccines as provided herein, in some embodiments, encode fusion proteins that comprise coronavirus antigens linked to one another or scaffold moieties. In some embodiments, such scaffold moieties impart desired properties to an antigen encoded by a nucleic acid of the disclosure. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 Å diameter, corresponding to 180 or 240 protomers. In some embodiments, the coronavirus antigen is fused to HBsAG or HBcAG to facilitate self-assembly of nanoparticles displaying the coronavirus antigen.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

In some embodiments, an RNA of the present disclosure encodes a coronavirus antigen fused to a foldon domain. The foldon domain may be, for example, obtained from bacteriophage T4 fibritin (see, e.g., Tao Y, et al. Structure. 1997 Jun. 15; 5(6):789-98).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker. In some embodiments, the linker is a GGGS linker (SEQ ID NO: 67). In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a coronavirus antigen). In some embodiments, a codon optimized sequence shares less than 90% sequence identity to a naturally-occurring or wild-type sequence (e.g., a naturally-occurring or wild-type mRNA sequence encoding a coronavirus antigen). In some embodiments, a codon optimized sequence shares less than 85% sequence identity to a naturally-occurring or wild-type sequence (e.g., a naturally-occurring or wild-type mRNA sequence encoding a coronavirus antigen). In some embodiments, a codon optimized sequence shares less than 80% sequence identity to a naturally-occurring or wild-type sequence (e.g., a naturally-occurring or wild-type mRNA sequence encoding a coronavirus antigen). In some embodiments, a codon optimized sequence shares less than 75% sequence identity to a naturally-occurring or wild-type sequence (e.g., a naturally-occurring or wild-type mRNA sequence encoding a coronavirus antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a coronavirus antigen). In some embodiments, a codon optimized sequence shares between 65% and 75% or about 80% sequence identity to a naturally-occurring or wild-type sequence (e.g., a naturally-occurring or wild-type mRNA sequence encoding a coronavirus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a coronavirus antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, an mRNA is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

The compositions of the present disclosure comprise, in some embodiments, an RNA having an open reading frame encoding a coronavirus antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (el w), 5-methoxy-uridine (mo5U), 5-methyl-cytidine (m5C), and/or pseudouridine (w). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (m1ψ) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (ψ) substitutions at one or more or all uridine positions of the nucleic acid.

In some embodiments, a mRNA of the disclosure comprises pseudouridine (ψ) substitutions at one or more or all uridine positions of the nucleic acid and 5-methyl cytidine substitutions at one or more or all cytidine positions of the nucleic acid.

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 37), where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 38) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (1743) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises a sequence selected from SEQ ID NO: 2 and SEQ ID NO: 33.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### Non-Coding Sequences

Aspects of the disclosure relate to multivalent RNA compositions which comprise mRNAs, e.g., 2-15 mRNA polynucleotides each comprising a distinct open reading frame (ORF) encoding a coronavirus virus antigenic polypeptide, wherein each mRNA polynucleotide comprises one or more non-coding sequences in an untranslated region (UTR) having unique identifier sequences (non-coding sequences). In some embodiments the non-coding sequence is a unique non-coding sequence. In some embodiments, each mRNA in a multivalent vaccine composition comprises its own unique non-coding sequence. As used herein, “non-coding sequence” refers to a sequence of a biological molecule (e.g., nucleic acid, protein, etc.) that when combined with the sequence another biological molecule serves to identify the other biological molecule. Typically, a non-coding sequence is a heterologous sequence that is incorporated within or appended to a sequence of a target biological molecule and utilized as a reference in order to identify a target molecule of interest. In some embodiments, a non-coding sequence is a sequence of a nucleic acid (e.g., a heterologous or synthetic nucleic acid) that is incorporated within or appended to a target nucleic acid and utilized as a reference in order to identify the target nucleic acid. In some embodiments, a non-coding sequence is of the formula (N)n. In some embodiments, n is an integer in the range of 5 to 20, 5 to 10, 10 to 20, 7 to 20, or 7 to 30. In some embodiments, n is 2, 3, 4, 5, 6, 7, 8, 9, 10, 11, 12, 13, 14, 15, 16, 17, 18, 19, 20, 21, 22, 23, 24, 25, 26, 27, 28, 29, 30 or more. In some embodiments, each N is a nucleotide that is independently selected from A, G, T, U, and C, or analogues thereof. Thus, some embodiments comprise nucleic acids (e.g., mRNAs) that (i) have a target sequence of interest (e.g., a coding sequence (e.g., that encodes an antigen protein or antigenic polypeptide)); and (ii) comprises a unique non-coding sequence.

In some embodiments, one or more in vitro transcribed mRNAs comprise one or more non-coding sequences in an untranslated region (UTR), such as a 5′ UTR or 3′ UTR. Inclusion of a non-coding sequence in the UTR of an mRNA prevents non-coding sequence from being translated into a peptide. In some embodiments, a non-coding sequence is positioned in a 3′ UTR of an mRNA. In some embodiments, the non-coding sequence is positioned upstream of the polyA tail of the mRNA. In some embodiments, the non-coding sequence is positioned downstream of (e.g., after) the polyA tail of the mRNA. In some embodiments, the non-coding sequence is positioned between the last codon of the ORF of the mRNA and the first “A” of the polyA tail of the mRNA. In some embodiments, a polynucleotide non-coding sequence positioned in a UTR comprises between 1 and 10 nucleotides (e.g., 1, 2, 3, 4, 5, 6, 7, 8, 9, or 10 nucleotides).

In some embodiments, UTR comprising a polynucleotide non-coding sequence further comprises one or more (e.g., 1, 2, 3, or more) RNAse cleavage sites, such as RNase H cleavage sites. In some embodiments, each different RNA of a multivalent RNA composition comprises a different (e.g., unique) non-coding sequence. In some embodiments, RNAs of a multivalent RNA composition are detected and/or purified according to the polynucleotide non-coding sequences of the RNAs. In some embodiments, the mRNA non-coding sequences are used to identify the presence of mRNA or determine a relative ratio of different mRNAs in a sample (e.g., a reaction product or a drug product). In some embodiments, the mRNA non-coding sequences are detected using one or more of deep sequencing, PCR, and Sanger sequencing. Exemplary non-coding sequences include: AACGUGAU; AAACAUCG; ATGCCUAA; AGUGGUCA; ACCACUGU; ACAUUGGC; CAGAUCUG; CAUCAAGU; CGCUGAUC; ACAAGCUA; CUGUAGCC; AGUACAAG; AACAACCA; AACCGAGA; AACGCUUA; AAGACGGA; AAGGUACA; ACACAGAA; ACAGCAGA; ACCUCCAA; ACGCUCGA; ACGUAUCA; ACUAUGCA; AGAGUCAA; AGAUCGCA; AGCAGGAA; AGUCACUA; AUCCUGUA; AUUGAGGA; CAACCACA; GACUAGUA; CAAUGGAA; CACUUCGA; CAGCGUUA; CAUACCAA; CCAGUUCA; CCGAAGUA; ACAGUG; CGAUGU; UUAGGC; AUCACG; and UGACCA.

In some embodiments the multivalent RNA composition is produced by a method comprising:
(a) combining a linearized first DNA molecule encoding the first mRNA polynucleotide, a linearized second DNA molecule encoding the second mRNA polynucleotide, and, optionally, a linearized third, fourth, fifth, sixth, seventh, eighth, ninth or tenth DNA molecule encoding the third, fourth, fifth, sixth, seventh, eighth, ninth or tenth mRNA polynucleotide into a single reaction vessel, wherein the first DNA molecule, the second DNA molecule, and the third, fourth, fifth, sixth, seventh, eighth, ninth or tenth DNA molecule are obtained from different sources; and
(b) simultaneously in vitro transcribing the linearized first DNA molecule, the linearized second DNA molecule and the optional linearized third, fourth, fifth, sixth, seventh, eighth, ninth or tenth DNA molecule to obtain a multivalent RNA composition. The different sources may be bacterial cell cultures which may not be co-cultured. In some embodiments the amounts of the first, second and third, fourth, fifth, sixth, seventh, eighth, ninth or tenth DNA molecules present in the reaction mixture prior to the start of the IVT have been normalized.

### In Vitro Transcription of RNA

cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to coronavirus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Nanoparticles (LNPs)

In some embodiments, the mRNA of the disclosure is formulated in a lipid nanoparticle (LNP). It is to be understood that “a lipid nanoparticle,” as used herein refers to a single LNP or a population of LNPs. Lipid nanoparticles typically comprise ionizable amino (cationic) lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises 20-60 mol % ionizable amino lipid. In some embodiments, the lipid nanoparticle comprises 20-55 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 40 mol %, 41 mol %, 42 mol %, 43 mol %, 44 mol % 45 mol %, 46 mol %, 47 mol %, 48 mol %, 49 mol %, 50 mol %, or 60 mol % ionizable amino lipid.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

In some embodiments, the lipid nanoparticle comprises 20-60 mol % ionizable amino lipid, 5-25 mol % non-cationic lipid, 25-55 mol % sterol, and 0.5-15 mol % PEG-modified lipid. In some embodiments, the lipid nanoparticle comprises 40-50 mol % ionizable amino lipid, 5-15 mol % neutral lipid, 20-40 mol % cholesterol, and 0.5-3 mol % PEG-modified lipid. In some embodiments, the lipid nanoparticle comprises 45-50 mol % ionizable amino lipid, 9-13 mol % neutral lipid, 35-45 mol % cholesterol, and 2-3 mol % PEG-modified lipid. In some embodiments, the lipid nanoparticle comprises 48 mol % ionizable amino lipid, 11 mol % neutral lipid, 68.5 mol % cholesterol, and 2.5 mol % PEG-modified lipid.

In some embodiments, an ionizable amino lipid of the disclosure comprises a compound of Formula (I):

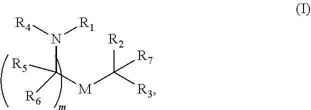

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, a subset of compounds of Formula (I) includes those of Formula (IA):

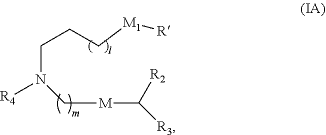

or a salt or isomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

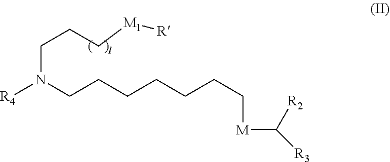

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

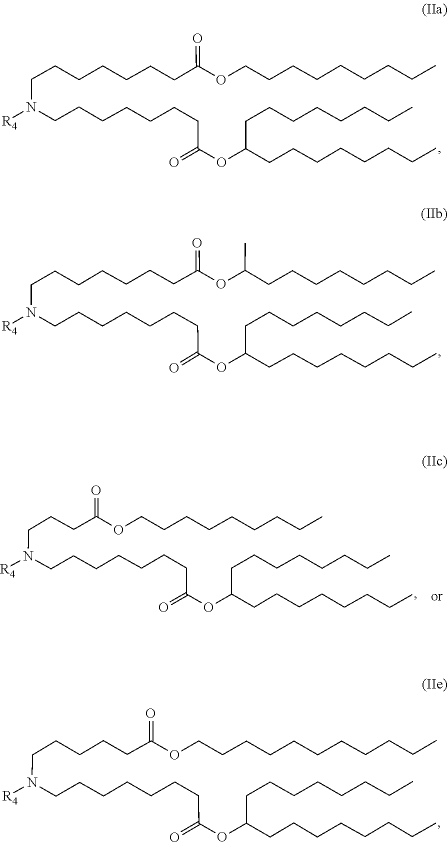

or a salt or isomer thereof, wherein R₄ is as described herein.

In some embodiments, a subset of compounds of Formula (I) includes those of Formula (IId):

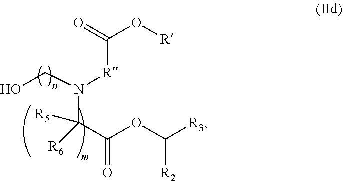

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

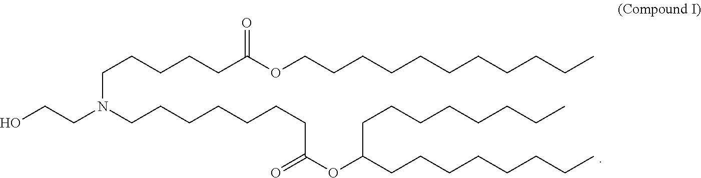 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

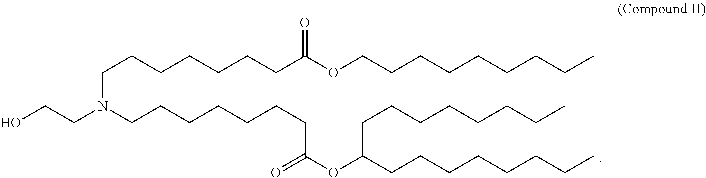 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable amino lipid of Compound 1, wherein the non-cationic lipid is DSPC, the structural lipid that is cholesterol, and the PEG lipid is DMG-PEG (e.g., PEG2000-DMG).

In some embodiments, the lipid nanoparticle comprises 45-55 mole percent (mol %) ionizable amino lipid (e.g., Compound 1). For example, lipid nanoparticle may comprise 45-47, 45-48, 45-49, 45-50, 45-52, 46-48, 46-49, 46-50, 46-52, 46-55, 47-48, 47-49, 47-50, 47-52, 47-55, 48-50, 48-52, 48-55, 49-50, 49-52, 49-55, or 50-55 mol % ionizable amino lipid (e.g., Compound 1). For example, lipid nanoparticle may comprise 45, 46, 47, 48, 49, 50, 51, 52, 53, 54, or 55 mol % ionizable amino lipid.

In some embodiments, the lipid nanoparticle comprises 5-15 mol % non-cationic (neutral) lipid (e.g., DSPC). For example, the lipid nanoparticle may comprise 5-6, 5-7, 5-8, 5-9, 5-10, 5-11, 5-12, 5-13, 5-14, 5-15, 6-7, 6-8, 6-9, 6-10, 6-11, 6-12, 6-13, 6-14, 6-15, 7-8, 7-9, 7-10, 7-11, 7-12, 7-13, 7-14, 7-15, 8-9, 8-10, 8-11, 8-12, 8-13, 8-14, 8-15, 9-10, 9-11, 9-12, 9-13, 9-14, 9-15, 10-11, 10-12, 10-13, 10-14, 10-15, 11-12, 11-13, 11-14, 11-15, 12-13, 12-14, 13-14, 13-15, or 14-15 mol % non-cationic (neutral) lipid (e.g., DSPC). For example, the lipid nanoparticle may comprise 5, 6, 7, 8, 9, 10, 11, 12, 13, 14, or 15 mol % DSPC.

In some embodiments, the lipid nanoparticle comprises 35-40 mol % sterol (e.g., cholesterol). For example, the lipid nanoparticle may comprise 35-36, 35-37, 35-38, 35-39, 35-40, 36-37, 36-38, 36-39, 36-40, 37-38, 37-39, 37-40, 38-39, 38-40, or 39-40 mol % cholesterol. For example, the lipid nanoparticle may comprise 35, 35.5, 36, 36.5, 37, 37.5, 38, 38.5, 39, 39.5, or 40 mol % cholesterol.

In some embodiments, the lipid nanoparticle comprises 1-3 mol % DMG-PEG. For example, the lipid nanoparticle may comprise 1-1.5, 1-2, 1-2.5, 1-3, 1.5-2, 1.5-2.5, 1.5-3, 2-2.5, 2-3, or 2.5-3. mol % DMG-PEG. For example, the lipid nanoparticle may comprise 1, 1.5, 2, 2.5, or 3 mol % DMG-PEG.

In some embodiments, the lipid nanoparticle comprises 50 mol % ionizable amino lipid, 10 mol % DSPC, 38.5 mol % cholesterol, and 1.5 mol % DMG-PEG. In some embodiments, the lipid nanoparticle comprises 48 mol % ionizable amino lipid, 11 mol % DSPC, 38.5 mol % cholesterol, and 2.5 mol % PEG2000-DMG.

In some embodiments, an LNP of the disclosure comprises an N:P ratio of from about 2:1 to about 30:1.

In some embodiments, an LNP of the disclosure comprises an N:P ratio of about 6:1.

In some embodiments, an LNP of the disclosure comprises an N:P ratio of about 3:1.

In some embodiments, an LNP of the disclosure comprises a wt/wt ratio of the ionizable amino lipid component to the RNA of from about 10:1 to about 100:1.

In some embodiments, an LNP of the disclosure comprises a wt/wt ratio of the ionizable amino lipid component to the RNA of about 20:1.

In some embodiments, an LNP of the disclosure comprises a wt/wt ratio of the ionizable amino lipid component to the RNA of about 10:1.

In some embodiments, an LNP of the disclosure has a mean diameter from about 50 nm to about 150 nm.

In some embodiments, an LNP of the disclosure has a mean diameter from about 70 nm to about 120 nm.

### Multivalent Vaccines

The compositions, as provided herein, may include RNA or multiple RNAs encoding two or more antigens of the same or different species. In some embodiments, composition includes an RNA or multiple RNAs encoding two or more coronavirus antigens. In some embodiments, the RNA may encode 1, 2, 3, 4, 5, 6, 7, 8, 9, or 10, or more coronavirus antigens.

In some embodiments, two or more different mRNA encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately. In some embodiments, when the composition comprises two different RNA encoding antigens, the ratio of RNA encoding antigens is 1:1, 1:2, 1:4, 4:1, or 2:1.

### Initial or First Vaccine

In some embodiments the first or initial vaccine is an mRNA vaccine encoding an antigen from a first circulating strain of SARS-CoV-2 virus. For instance, the initial or first vaccine may be an mRNA encoding a spike antigen having an amino acid sequence of SEQ ID NO: 36, domain or subunit thereof. In other embodiments the first vaccine may be any vaccine modality comprising a corresponding antigen from a second or subsequent circulating strain of SARS-CoV-2 virus. As a non-limiting example, the first vaccine composition may be a recombinant vaccine. As used herein, “recombinant vaccine” refers to a vaccine made by genetic engineering, the process and method of manipulating the genetic material of an organism. In most cases, a recombinant vaccine encompasses one or more nucleic acids encoding protein antigens that have either been produced and purified in a heterologous expression system (e.g., bacteria or yeast) or purified from large amounts of the pathogenic organism. Following administration, a vaccinated person produces antibodies to the one or more protein antigens, thus protecting him/her from disease. In some embodiments, the recombinant vaccine is a vectored vaccine. Viral vectored vaccines comprise a polynucleotide sequence not of viral origin (i.e., a polynucleotide heterologous to the virus), that encodes a peptide, polypeptide, or protein capable of eliciting an immune response in a host contacted with the vector. Expression of the polynucleotide results in the generation of a neutralizing antibody response and/or a cell mediated response, e.g., a cytotoxic T cell response. Examples of viral vectored vaccines include, but are not limited to, those developed by Oxford/AstraZeneca (COVID-19 Vaccine AstraZeneca), CanSino Biological Inc./Beijing Institute of Biotechnology, Gamaleya Research Institute, Zydus Cadila, Institut Pasteur/Themis/University of Pittsburgh Center for Vaccine Research, University of Hong Kong, and Altimmune (NasoVAX). In some embodiments, the recombinant vaccine is a nucleic acid-based (e.g., DNA, mRNA) coronavirus vaccine. Exemplary DNA vaccines include those being developed by Inovio Pharmaceuticals (INO-4800), Genexine Consortium (GX-19), OncoSec and the Cancer Institute (CORVax12 and TAVO™), Karolinska Institute/Cobra Biologics, Osaka University/Anges/Takara Bio, and Takis/Applied DNA Sciences/Evvivax. Exemplary mRNA vaccines include those being developed by BioNTech/Pfizer, Imperial College London, Curevac, and Walvax Biotech/People's Liberation Army (PLA) Academy of Military Science.

### Pharmaceutical Formulations

Provided herein are compositions (e.g., pharmaceutical compositions), methods, kits and reagents for prevention or treatment of coronavirus in humans and other mammals, for example. The compositions provided herein can be used as therapeutic or prophylactic agents. They may be used in medicine to prevent and/or treat a coronavirus infection.

In some embodiments, the SARS-CoV-2 vaccine containing RNA as described herein can be administered to a subject (e.g., a mammalian subject, such as a human subject), and the RNA polynucleotides are translated in vivo to produce an antigenic polypeptide (antigen).

An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of a composition provides an induced or boosted immune response as a function of antigen production in the cells of the subject. In some embodiments, an effective amount of the composition containing RNA polynucleotides having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the compositions (comprising polynucleotides and their encoded polypeptides) in accordance with the present disclosure may be used for treatment or prevention of a coronavirus infection. A composition may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, or 6 months, 7 months, 8 months, 9 months, 10 months, 11 months, one year, or more. In some embodiments, the time of administration between the initial administration of the prophylactic composition and the booster is at least 6 months. In exemplary embodiments, the time of administration between the initial administration of the prophylactic composition and the booster may be, but is not limited to, 1 week, 2 weeks, 3 weeks, 1 month, 2 months, 3 months, or 6 months. As is described herein, the booster may comprise the same or different mRNAs as compared to the earlier administration of the prophylactic composition. In some embodiments, the booster may comprise a combination of the same mRNA from the earlier administration of the prophylactic composition and at least one different mRNA. In some embodiments, the ratio of the mRNA from the earlier administration of the prophylactic composition and the at least one different mRNA is 1:1, 1:2, 1:4, 4:1, or 2:1. In one embodiment, the ratio is 1:1. The booster, in some embodiments is monovalent (e.g., the mRNA encodes a single antigen). In some embodiments, the booster is multivalent (e.g., the mRNA encodes more than one antigen).

In some embodiments, the booster dose is 5 μg-30 μg, 5 μg-25 μg, 5 μg-20 μg, 5 μg-15 μg, 5 μg-10 μg, 10 μg-30 μg, 10 μg-25 μg, 10 μg-20 μg, 10 μg-15 μg, 15 μg-30 μg, 15 μg-25 μg, 15 μg-20 μg, 20 μg-30 μg, 25 μg-30 μg, or 25 μg-300 μg. In some embodiments, the booster dose is 10 μg-60 μg, 10 μg-55 μg, 10 μg-50 μg, 10 μg-45 μg, 10 μg-40 μg, 10 μg-35 μg, 10 μg-30 μg, 10 μg-25 μg, 10 μg-20 μg, 15 μg-60 μg, 15 μg-55 μg, 15 μg-50 μg, 15 μg-45 μg, 15 μg-40 μg, 15 μg-35 μg, 15 μg-30 μg, 15 μg-25 μg, 15 μg-20 μg, 20 μg-60 μg, 20 μg-55 μg, 20 μg-50 μg, 20 μg-45 μg, 20 μg-40 μg, 20 μg-35 μg, 20 μg-30 μg, 20 μg-25 μg, 25 μg-60 μg, 25 μg-55 μg, 25 μg-50 μg, 25 μg-45 μg, 25 μg-40 μg, 25 μg-35 μg, 25 μg-30 μg, 30 μg-60 μg, 30 μg-55 μg, 30 μg-50 μg, 30 μg-45 μg, 30 μg-40 μg, 30 μg-35 μg, 35 μg-60 μg, 35 μg-55 μg, 35 μg-50 μg, 35 μg-45 μg, 35 μg-40 μg, 40 μg-60 μg, 40 μg-55 μg, 40 μg-50 μg, 40 μg-45 μg, 45 μg-60 μg, 45 μg-55 μg, 45 μg-50 μg, 50 μg-60 μg, 50 μg-55 μg, or 55 μg-60 μg. In some embodiments, the booster dose is at least 10 μg and less than 25 μg of the composition. In some embodiments, the booster dose is at least 5 μg and less than 25 μg of the composition. For example, the booster dose is 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the booster dose is 50 μg.

In some embodiments, a composition may be administered intramuscularly, intranasally or intradermally, similarly to the administration of inactivated vaccines known in the art.

A composition may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including RNA and/or complexes optionally in combination with one or more pharmaceutically acceptable excipients.

The RNA may be formulated or administered alone or in conjunction with one or more other components. For example, an immunizing composition may comprise other components including, but not limited to, adjuvants.

In some embodiments, an immunizing composition does not include an adjuvant (they are adjuvant free).

An RNA may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, an immunizing composition is administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigens.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an RNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof.

### Dosing/Administration

Provided herein are immunizing compositions (e.g., RNA vaccines), methods, kits and reagents for prevention and/or treatment of coronavirus infection in humans and other mammals. Immunizing compositions can be used as therapeutic or prophylactic agents. In some embodiments, immunizing compositions are used to provide prophylactic protection from coronavirus infection. In some embodiments, immunizing compositions are used to treat a coronavirus infection. In some embodiments, embodiments, immunizing compositions are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, an immunizing composition (e.g., RNA vaccine) is administered to a subject (e.g., a mammalian subject, such as a human subject) in an effective amount to induce an antigen-specific immune response. The RNA encoding the coronavirus spike protein antigen is expressed and translated in vivo to produce the antigen, which then stimulates an immune response in the subject.

Prophylactic protection from a coronavirus can be achieved following administration of an immunizing composition (e.g., an RNA vaccine) of the present disclosure. Immunizing compositions can be administered once, twice, three times, four times or more but it is likely sufficient to administer the vaccine once (optionally followed by a single booster). It is possible, although less desirable, to administer an immunizing composition to an infected individual to achieve a therapeutic response. Dosing may need to be adjusted accordingly.

A method of eliciting an immune response in a subject against a coronavirus antigen (or multiple antigens) is provided in aspects of the present disclosure. In some embodiments, a method involves administering to the subject an immunizing composition comprising a mRNA having an open reading frame encoding a coronavirus antigen, thereby inducing in the subject an immune response specific to the coronavirus antigen, wherein anti-antigen antibody titer in the subject is increased following vaccination relative to anti-antigen antibody titer in a subject vaccinated with a prophylactically effective dose of a traditional vaccine against the antigen. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the coronavirus or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the coronavirus or an unvaccinated subject.

A method of eliciting an immune response in a subject against a coronavirus is provided in other aspects of the disclosure. The method involves administering to the subject a composition comprising an mRNA comprising an open reading frame encoding a coronavirus antigen, thereby inducing in the subject an immune response specific to the coronavirus, wherein the immune response in the subject is equivalent to an immune response in a subject vaccinated with a traditional vaccine against the coronavirus at 2 times to 100 times the dosage level relative to the composition.

In some embodiments, the immune response in the subject is equivalent to an immune response in a subject vaccinated with a traditional vaccine at twice the dosage level relative to a composition of the present disclosure. In some embodiments, the immune response in the subject is equivalent to an immune response in a subject vaccinated with a traditional vaccine at three times the dosage level relative to a composition of the present disclosure. In some embodiments, the immune response in the subject is equivalent to an immune response in a subject vaccinated with a traditional vaccine at 4 times, 5 times, 10 times, 50 times, or 100 times the dosage level relative to a composition of the present disclosure. In some embodiments, the immune response in the subject is equivalent to an immune response in a subject vaccinated with a traditional vaccine at 10 times to 1000 times the dosage level relative to a composition of the present disclosure. In some embodiments, the immune response in the subject is equivalent to an immune response in a subject vaccinated with a traditional vaccine at 100 times to 1000 times the dosage level relative to a composition of the present disclosure.

In other embodiments, the immune response is assessed by determining [protein] antibody titer in the subject. In other embodiments, the ability of serum or antibody from an immunized subject is tested for its ability to neutralize viral uptake or reduce coronavirus transformation of human B lymphocytes. In other embodiments, the ability to promote a robust T cell response(s) is measured using art recognized techniques.

Other aspects the disclosure provide methods of eliciting an immune response in a subject against a coronavirus by administering to the subject composition comprising an mRNA having an open reading frame encoding a coronavirus antigen, thereby inducing in the subject an immune response specific to the coronavirus antigen, wherein the immune response in the subject is induced 2 days to 10 weeks earlier relative to an immune response induced in a subject vaccinated with a prophylactically effective dose of a traditional vaccine against the coronavirus. In some embodiments, the immune response in the subject is induced in a subject vaccinated with a prophylactically effective dose of a traditional vaccine at 2 times to 100 times the dosage level relative to a composition of the present disclosure.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against a coronavirus by administering to the subject an mRNA having an open reading frame encoding a first antigen, wherein the RNA does not include a stabilization element, and wherein an adjuvant is not co-formulated or co-administered with the vaccine.

A composition may be administered by any route that results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. The RNA is typically formulated in dosage unit form for ease of administration and uniformity of dosage. It will be understood, however, that the total daily usage of the RNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the RNA (e.g., an effective dose), as provided herein, may be as low as 20 μg, administered for example as a single dose or as two 10 μg doses (e.g., a first effective vaccine dose and a second effective vaccine dose). In some embodiments, the first effective vaccine dose and the second effective vaccine dose are the same amount. In some embodiments, the first effective vaccine dose and the second effective vaccine dose are different amounts. In some embodiments, the effective amount is a total dose of 5 μg-30 μg, 5 μg-25 μg, 5 μg-20 μg, 5 μg-15 μg, 5 μg-10 μg, 10 μg-30 μg, 10 μg-25 μg, 10 μg-20 μg, 10 μg-15 μg, 15 μg-30 μg, 15 μg-25 μg, 15 μg-20 μg, 20 μg-30 μg, 25 μg-30 μg, or 25 μg-300 μg. In some embodiments, the effective amount is a total dose of 10 μg-60 μg, 10 μg-55 μg, 10 μg-50 μg, 10 μg-45 μg, 10 μg-40 μg, 10 μg-35 μg, 10 μg-30 μg, 10 μg-25 μg, 10 μg-20 μg, 15 μg-60 μg, 15 μg-55 μg, 15 μg-50 μg, 15 μg-45 μg, 15 μg-40 μg, 15 μg-35 μg, 15 μg-30 μg, 15 μg-25 μg, 15 μg-20 μg, 20 μg-60 μg, 20 μg-55 μg, 20 μg-50 μg, 20 μg-45 μg, 20 μg-40 μg, 20 μg-35 μg, 20 μg-30 μg, 20 μg-25 μg, 25 μg-60 μg, 25 μg-55 μg, 25 μg-50 μg, 25 μg-45 μg, 25 μg-40 μg, 25 μg-35 μg, 25 μg-30 μg, 30 μg-60 μg, 30 μg-55 μg, 30 μg-50 μg, 30 μg-45 μg, 30 μg-40 μg, 30 μg-35 μg, 35 μg-60 μg, 35 μg-55 μg, 35 μg-50 μg, 35 μg-45 μg, 35 μg-40 μg, 40 μg-60 μg, 40 μg-55 μg, 40 μg-50 μg, 40 μg-45 μg, 45 μg-60 μg, 45 μg-55 μg, 45 μg-50 μg, 50 μg-60 μg, 50 μg-55 μg, or 55 μg-60 μg. In some embodiments, the effective dose (e.g., effective amount) is at least 10 μg and less than 25 μg of the composition. In some embodiments, the effective dose (e.g., effective amount) is at least 5 μg and less than 25 μg of the composition. For example, the effective amount may be a total dose of 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the effective amount (e.g., effective dose) is a total dose of 10 μg. In some embodiments, the effective amount is a total dose of 20 μg (e.g., two 10 μg doses). In some embodiments, the effective amount is a total dose of 25 μg. In some embodiments, the effective amount is a total dose of 30 μg. In some embodiments, the effective amount is a total dose of 50 μg. In some embodiments, the effective amount is a total dose of 60 μg (e.g., two 30 μg doses). In some embodiments, the effective amount is a total dose of 75 μg. In some embodiments, the effective amount is a total dose of 100 μg. In some embodiments, the effective amount is a total dose of 150 μg. In some embodiments, the effective amount is a total dose of 200 μg. In some embodiments, the effective amount is a total dose of 250 μg. In some embodiments, the effective amount is a total dose of 300 μg. In some embodiments, the composition comprises two or more mRNA polynucleotides and effective amount is a total dose of 20 μg (e.g., 10 μg of a first mRNA and 10 μg of a second mRNA). In some embodiments, the composition comprises two or more mRNA polynucleotides and effective amount is a total dose of 50 μg (e.g., 25 μg of a first mRNA and 25 μg of a second mRNA). In some embodiments, the composition comprises two or more mRNA polynucleotides and effective amount is a total dose of 100 μg (e.g., 50 μg of a first mRNA and 50 μg of a second mRNA).

The RNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the compositions (e.g., RNA vaccines), wherein the RNA is formulated in an effective amount to produce an antigen specific immune response in a subject (e.g., production of antibodies specific to a coronavirus antigen). “An effective amount” is a dose of the RNA effective to produce an antigen-specific immune response. Also provided herein are methods of inducing an antigen-specific immune response in a subject.

As used herein, an immune response to a vaccine or LNP of the present disclosure is the development in a subject of a humoral and/or a cellular immune response to a (one or more) coronavirus protein(s) present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-coronavirus antigen antibody titer produced in a subject administered a composition as provided herein. An antibody titer is a measurement of the amount of antibodies within a subject, for example, antibodies that are specific to a particular antigen or epitope of an antigen. Antibody titer is typically expressed as the inverse of the greatest dilution that provides a positive result. Enzyme-linked immunosorbent assay (ELISA) is a common assay for determining antibody titers, for example.

A variety of serological tests can be used to measure antibody against encoded antigen of interest, for example, SAR-CoV-2 virus or SAR-CoV-2 viral antigen, e.g., SAR-CoV-2 spike or S protein, of domain thereof. These tests include the hemagglutination-inhibition test, complement fixation test, fluorescent antibody test, enzyme-linked immunosorbent assay (ELISA), and plaque reduction neutralization test (PRNT). Each of these tests measures different antibody activities. In exemplary embodiments, A plaque reduction neutralization test, or PRNT (e.g., PRNT50 or PRNT90) is used as a serological correlate of protection. PRNT measures the biological parameter of in vitro virus neutralization and is the most serologically virus-specific test among certain classes of viruses, correlating well to serum levels of protection from virus infection. The basic design of the PRNT allows for virus-antibody interaction to occur in a test tube or microtiter plate, and then measuring antibody effects on viral infectivity by plating the mixture on virus-susceptible cells, preferably cells of mammalian origin. The cells are overlaid with a semi-solid media that restricts spread of progeny virus. Each virus that initiates a productive infection produces a localized area of infection (a plaque), that can be detected in a variety of ways. Plaques are counted and compared back to the starting concentration of virus to determine the percent reduction in total virus infectivity. In PRNT, the serum sample being tested is usually subjected to serial dilutions prior to mixing with a standardized amount of virus. The concentration of virus is held constant such that, when added to susceptible cells and overlaid with semi-solid media, individual plaques can be discerned and counted. In this way, PRNT end-point titers can be calculated for each serum sample at any selected percent reduction of virus activity.

In functional assays intended to assess vaccinal immunogenicity, the serum sample dilution series for antibody titration should ideally start below the “seroprotective” threshold titer. Regarding SARS-CoV-2 neutralizing antibodies, the “seroprotective” threshold titer remains unknown; but a seropositivity threshold of 1:10 can be considered a seroprotection threshold in certain embodiments.

In some embodiments a neutralizing immune response is an immune response that produces a level of antibodies that meet or exceed a seroprotection threshold.

PRNT end-point titers are expressed as the reciprocal of the last serum dilution showing the desired percent reduction in plaque counts. The PRNT titer can be calculated based on a 50% or greater reduction in plaque counts (PRNT50). A PRNT50 titer is preferred over titers using higher cut-offs (e.g., PRNT90) for vaccine sera, providing more accurate results from the linear portion of the titration curve.

There are several ways to calculate PRNT titers. The simplest and most widely used way to calculate titers is to count plaques and report the titer as the reciprocal of the last serum dilution to show >50% reduction of the input plaque count as based on the back-titration of input plaques. Use of curve fitting methods from several serum dilutions may permit calculation of a more precise result. There are a variety of computer analysis programs available for this (e.g., SPSS or GraphPad Prism).

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by a composition (e.g., RNA vaccine).

In some embodiments, an anti-coronavirus antigen antibody titer produced in a subject is increased by at least 1 log relative to a control. For example, anti-coronavirus antigen antibody titer produced in a subject may be increased by at least 1.5, at least 2, at least 2.5, or at least 3 log relative to a control. In some embodiments, the anti-coronavirus antigen antibody titer produced in the subject is increased by 1, 1.5, 2, 2.5 or 3 log relative to a control. In some embodiments, the anti-coronavirus antigen antibody titer produced in the subject is increased by 1-3 log relative to a control. For example, the anti-coronavirus antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-coronavirus antigen antibody titer produced in a subject is increased at least 2 times relative to a control. For example, the anti-coronavirus antigen n antibody titer produced in a subject may be increased at least 3 times, at least 4 times, at least 5 times, at least 6 times, at least 7 times, at least 8 times, at least 9 times, or at least 10 times relative to a control. In some embodiments, the anti-coronavirus antigen antibody titer produced in the subject is increased 2, 3, 4, 5, 6, 7, 8, 9, or 10 times relative to a control. In some embodiments, the anti-coronavirus antigen antibody titer produced in a subject is increased 2-10 times relative to a control. For example, the anti-coronavirus antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to coronavirus. A geometric mean titer (GMT) is the average antibody titer for a group of subjects calculated by multiplying all values and taking the nth root of the number, where n is the number of subjects with available data.

A control, in some embodiments, is an anti-coronavirus antigen antibody titer produced in a subject who has not been administered a composition (e.g., RNA vaccine). In some embodiments, a control is an anti-coronavirus antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of a composition (e.g., RNA vaccine) to be effective is measured in a murine model. For example, a composition may be administered to a murine model and the murine model assayed for induction of neutralizing antibody titers. Viral challenge studies may also be used to assess the efficacy of a vaccine of the present disclosure. For example, a composition may be administered to a murine model, the murine model challenged with virus, and the murine model assayed for survival and/or immune response (e.g., neutralizing antibody response, T cell response (e.g., cytokine response)).

In some embodiments, an effective amount of a composition (e.g., RNA vaccine) is a dose that is reduced compared to the standard of care dose of a recombinant protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified protein vaccine, or a live attenuated or inactivated vaccine, or a VLP vaccine, that a physician/clinician or other medical professional would administer to a subject to treat or prevent coronavirus infection or a related condition, while following the standard of care guideline for treating or preventing coronavirus infection or a related condition.

In some embodiments, the anti-coronavirus antigen antibody titer produced in a subject administered an effective amount of an composition is equivalent to an anti-coronavirus antigen antibody titer produced in a control subject administered a standard of care dose of a recombinant or purified protein vaccine, or a live attenuated or inactivated vaccine, or a VLP vaccine.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, efficacy of the composition (e.g., RNA vaccine) is at least 60% relative to unvaccinated control subjects. For example, efficacy of the composition may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of a composition of the present disclosure is sufficient to provide sterilizing immunity in the subject for at least 1 year. For example, the effective amount of a composition of the present disclosure is sufficient to provide sterilizing immunity in the subject for at least 2 years, at least 3 years, at least 4 years, or at least 5 years. In some embodiments, the effective amount of a composition of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of a composition of the present disclosure is sufficient to produce detectable levels of coronavirus antigen as measured in serum of the subject at 1-72 hours post administration.

Titer. An antibody titer is a measurement of the number of antibodies within a subject, for example, antibodies that are specific to a particular antigen (e.g., an anti-coronavirus antigen). Antibody titer is typically expressed as the inverse of the greatest dilution that provides a positive result. Enzyme-linked immunosorbent assay (ELISA) is a common assay for determining antibody titers, for example.

A neutralizing immune response is an immune response that is a neutralizing antibody response and/or an effective neutralizing T cell response. In some embodiments a neutralizing antibody response produces a level of antibodies that meet or exceed a seroprotection threshold.

An effective T cell response is a response which produces a baseline level of viral activated or viral specific T cells including CD8+ and CD4+ T helper type 1 cells. CD8+ cytotoxic T lymphocytes typically clear the intracellular virus compartment and CD4+ T cells exert various functions in the body such as helping B and other T cells, promoting memory generation and indirect or direct cytotoxic activity. In some embodiments the effective T cells comprises a high proportion of CD8+ T cells and/or CD4+ T cells, relative to a baseline level (in a naïve subject). In some embodiments these T cells are differentiated towards an early-differentiated memory phenotype with co-expression of CD27 and CD28.

In some embodiments, the effective amount of a composition of the present disclosure is sufficient to produce a 1,000-10,000 neutralizing antibody titer produced by neutralizing antibody against the coronavirus antigen as measured in serum of the subject at 1-72 hours post administration. In some embodiments, the effective amount is sufficient to produce a 1,000-5,000 neutralizing antibody titer produced by neutralizing antibody against the coronavirus antigen as measured in serum of the subject at 1-72 hours post administration. In some embodiments, the effective amount is sufficient to produce a 5,000-10,000 neutralizing antibody titer produced by neutralizing antibody against the coronavirus antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-coronavirus antigen antibody titer produced in the subject is increased by at least 1 log relative to a control. For example, an anti-coronavirus antigen antibody titer produced in the subject may be increased by at least 2, 3, 4, 5, 6, 7, 8, 9 or 10 log relative to a control.

In some embodiments, an anti-coronavirus antigen antibody titer produced in the subject is increased at least 2 times relative to a control. For example, an anti-coronavirus antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated viral vaccine, an inactivated viral vaccine, or a protein subunit vaccine.

### Additional Embodiments

1. A nucleic acid encoding a SARS-CoV-2 antigen of a second circulating SARS-CoV-2 virus strain, wherein the antigen has an amino acid sequence with at least one amino acid mutation with respect to an antigen of a first circulating SARS-CoV-2 virus strain, wherein the mutation is an amino acid substitution, deletion or insertion, wherein the antigen is not a full length stabilized spike protein having a 2P mutation and wherein the nucleic acid is in a lipid nanoparticle.

2. The nucleic acid of paragraph 1, wherein the first and second virus strains are in circulation for at least a portion of 1 year.

3. The nucleic acid of paragraph 1, wherein the first and second virus strains are in circulation during the same pandemic period or endemic period.

4. The nucleic acid of any one of paragraphs 1-3, wherein the SARS-CoV-2 antigen has a second amino acid mutation with respect to the antigen of the first circulating SARS-CoV-2 virus strain, wherein the mutation is an amino acid substitution, deletion or insertion, and the second mutation corresponds to a third virus strain in circulation.

5. The nucleic acid of any one of paragraphs 1-4, wherein the lipid nanoparticle comprises an ionizable amino lipid, a sterol, a neutral lipid, and a polyethylene glycol (PEG)-modified lipid.

6. The nucleic acid of paragraph 5, wherein the lipid nanoparticle comprises 40-55 mol % ionizable amino lipid, 30-45 mol % sterol, 5-15 mol % neutral lipid, and 1-5 mol % PEG-modified lipid.

7. The nucleic acid of any one of paragraphs 1-6, wherein the SARS-CoV-2 antigen is a receptor binding domain (RBD) of spike protein.

8. The nucleic acid of any one of paragraphs 1-6, wherein the SARS-CoV-2 antigen is an N-terminal domain (NTD).

9. The nucleic acid of any one of paragraphs 1-6, wherein the SARS-CoV-2 antigen is a combination of an RBD and NTD.

10. The nucleic acid of any one of paragraphs 1-6, wherein the SARS-CoV-2 antigen is an NTD, RBD, and transmembrane domain joined by linkers.

11. The nucleic acid of paragraph 10, wherein the SARS-CoV-2 antigen is an NTD, RBD, and influenza hemagglutinin transmembrane (HATM) domain joined by linkers (NTD-RBD-HATM).

12. The nucleic acid of any one of paragraphs 1-6, wherein the SARS-CoV-2 antigen is an NTD-RBD fusion.

13. The nucleic acid of any one of paragraphs 1-6, wherein the SARS-CoV-2 antigen is an S1 protein subunit.

14. The nucleic acid of any one of paragraphs 1-13, wherein the amino acid mutation in the antigen is associated with a structure or function of the virus that is different in quantity or kind from a corresponding structure or function in the first virus.

15. The nucleic acid of paragraph 14, wherein the function is ACE2 receptor binding, virus transmissibility, viral uptake, viral pathogenesis, altered furin cleavage, or viral replication.

16. The nucleic acid of any one of paragraphs 1-15, wherein the nucleic acid encodes a protein having at least 95% sequence identity to a protein of any one of SEQ ID NOs: 20, 23, 26, 29, 32, 57, 60, and 63.

17. The nucleic acid of any one of paragraphs 1-16, wherein the nucleic acid has at least 95% sequence identity to an RNA of any one of SEQ ID NOs: 21, 24, 27, 30, 55, 58, 61, and 64.

18. The nucleic acid of any one of paragraphs 1-17, wherein the nucleic acid comprises messenger RNA (mRNA).

19. A composition comprising: a first messenger ribonucleic acid (mRNA) encoding a first SARS-CoV-2 antigen of a first SARS-CoV-2 virus and a second mRNA encoding a second SARS-CoV-2 antigen of a second SARS-CoV-2 virus, wherein the second SARS-CoV-2 virus has an amino acid sequence with at least one amino acid mutation with respect to an amino acid sequence of the first SARS-CoV-2 virus, wherein the mutation is an amino acid substitution, deletion or insertion, wherein each of the first and second antigens are not full length stabilized spike proteins having a 2P mutation.

20. The composition of paragraph 19, wherein the first SARS-CoV-2 virus is a first circulating SARS-CoV-2 virus strain, and the second SARS-CoV-2 virus is a second circulating SARS-CoV-2 virus strain, and wherein the first and second virus strains are spreading in the population for at least a portion of 1 year.

21. The composition of any one of paragraphs 19-20, wherein the mRNAs are present at about a 1:1 ratio relative to each other mRNA in the composition.

22. The composition of any one of paragraphs 19-21, wherein the mRNAs are in a lipid nanoparticle and wherein the lipid nanoparticle comprises an ionizable amino lipid, a sterol, a neutral lipid, and a polyethylene glycol (PEG)-modified lipid.

23. The composition of paragraph 22, wherein the lipid nanoparticle comprises 40-55 mol % ionizable amino lipid, 30-45 mol % sterol, 5-15 mol % neutral lipid, and 1-5 mol % PEG-modified lipid.

24. The composition of any one of paragraphs 22-23, wherein the lipid nanoparticle comprises 40-50 mol % ionizable amino lipid, 35-45 mol % sterol, 10-15 mol % neutral lipid, and 2-4 mol % PEG-modified lipid.

25. The composition of any one of paragraphs 22-24, wherein the lipid nanoparticle comprises 45 mol %, 46 mol %, 47 mol %, 48 mol %, 49 mol %, or 50 mol % ionizable amino lipid.

26. The composition of any one of paragraphs 22-25, wherein the ionizable amino lipid has the structure of Compound 1:

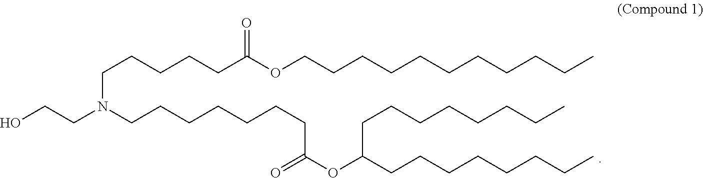 [SMILES: CCCCCCCCCCCOC(=O)CCCCCN(CCO)CCCCCCCC(=O)OC(CCCCCCCC)CCCCCCCC]

27. The composition of any one of paragraphs 22-26, wherein the sterol is cholesterol or a variant thereof.

28. The composition of any one of paragraphs 22-27, wherein the neutral lipid is 1,2 distearoyl-sn-glycero-3-phosphocholine (DSPC).

29. The composition of any one of paragraphs 22-28, wherein the PEG-modified lipid is 1,2 dimyristoyl-sn-glycerol, methoxypolyethyleneglycol (PEG2000 DMG).

30. An mRNA, wherein the mRNA encodes a protein having at least 95% sequence identity to a protein of any one of SEQ ID NOs: 20, 23, 26, 29, 32, 57, 60, and 63.

31. An mRNA, wherein the mRNA has at least 95% sequence identity to an RNA of any one of SEQ ID NOs: 21, 24, 27, 30, 55, 58, 61, and 64.

32. An mRNA, wherein the mRNA has at least 98% sequence identity to an RNA of any one of SEQ ID NOs: 21, 24, 27, 30, 55, 58, 61, and 64.

33. An mRNA, wherein the mRNA comprises an RNA of any one of SEQ ID NOs: 21, 24, 27, 30, 55, 58, 61, and 64.

34. A method comprising: administering to a subject a second effective dose of a second vaccine comprising a second nucleic acid encoding a second SARS-CoV-2 antigen, wherein the subject has previously been administered a first effective dose of a first vaccine comprising a first nucleic acid encoding a first SARS-CoV-2 antigen, wherein the second vaccine is administered in an effective amount to induce an immune response specific for the second antigen, wherein the second SARS-CoV-2 antigen has an amino acid sequence with at least one mutation, with respect to a corresponding amino acid sequence of the first SARS-CoV-2 antigen, wherein the mutation is an amino acid substitution, deletion or insertion and wherein each of the first and second antigens are not full length stabilized spike proteins having a 2P mutation.

35. The method of paragraph 34, wherein the first encoded SARS-CoV-2 antigen is of a first circulating SARS-CoV-2 virus and wherein the second encoded SARS-CoV-2 antigen is of a second circulating SARS-CoV-2 virus.

36. The method of paragraph 34, wherein the first encoded SARS-CoV-2 antigen is representative of a first circulating SARS-CoV-2 virus and wherein the second encoded SARS-CoV-2 antigen is representative of a second circulating SARS-CoV-2 virus.

37. The method of paragraph 34, wherein the first encoded SARS-CoV-2 antigen is representative of a plurality of first circulating SARS-CoV-2 viruses and/or wherein the second encoded SARS-CoV-2 antigen is representative of a second plurality of circulating SARS-CoV-2 viruses.

38. The method of paragraph 34, wherein the first encoded SARS-CoV-2 antigen is of a first circulating SARS-CoV-2 virus and wherein the second encoded SARS-CoV-2 antigen is of a second circulating SARS-CoV-2 virus.

39. The method of any one of paragraphs 35-38, wherein the second circulating SARS-CoV-2 virus is an immunodominant emerging strain or variant of concern detected during a period when the first circulating SARS-CoV-2 virus is present in a subject population.

40. The method of any one of paragraphs 35-39, wherein the second circulating SARS-CoV-2 virus and the first circulating SARS-CoV-2 virus are detectable in a subject population within at least one year.

41. The method of any one of paragraphs 35-39, wherein the second circulating SARS-CoV-2 virus and the first circulating SARS-CoV-2 virus are detectable in a subject population during a same season.

42. The method of any one of paragraphs 35-39, wherein the second circulating SARS-CoV-2 virus and the first circulating SARS-CoV-2 virus are detectable in a subject population during a same pandemic period or endemic period.

43. The method of any one of paragraphs 34-42, wherein the first nucleic acid encoding the first SARS-CoV-2 antigen is a DNA or a messenger RNA (mRNA).

44. The method of any one of paragraphs 34-43, wherein the first nucleic acid encoding a first SARS-CoV-2 antigen is a messenger RNA (mRNA).

45. The method of any one of paragraphs 34-43, wherein the second nucleic acid encoding a second SARS-CoV-2 antigen is a messenger RNA (mRNA).

46. The method of any one of paragraphs 34-45, wherein the second vaccine further comprises the first nucleic acid encoding a first SARS-CoV-2 antigen.

47. The method of paragraph 46, wherein the first nucleic acid encoding a first SARS-CoV-2 antigen and the second nucleic acid encoding a second SARS-CoV-2 antigen are present in the second vaccine in a 1:1 ratio.

48. The method of any one of paragraphs 34-45, wherein the first and/or second vaccine is a monovalent vaccine.

49. The method of any one of paragraphs 34-47 wherein the first and/or second vaccine is a multivalent vaccine, comprising more than one nucleic acids encoding antigens representative of a plurality of variants of concern.

50. The method of any one of paragraphs 34-49, wherein the first encoded SARS-CoV-2 antigen is administered to the subject as a first vaccine comprised of one or more doses and the second encoded SARS-CoV-2 antigen is administered to the subject as a boost in one or more doses.

51. The method of any one of paragraphs 34-49, wherein the second encoded SARS-CoV-2 antigen is administered to the subject as first vaccine comprised of one or more doses and the first encoded SARS-CoV-2 antigen is administered to the subject as a boost in one or more doses.

52. The method of any one of paragraphs 34-49, wherein the first and second encoded SARS-CoV-2 antigens are administered to the subject together as a boost dose, optionally wherein the first and second encoded SARS-CoV-2 antigens are present in the boost dose in a 1:1 ratio.

53. The method of any one of paragraphs 34-49, wherein the first encoded SARS-CoV-2 antigen is administered to the subject as a prime dose and as a boost dose to complete a vaccination and wherein the second encoded SARS-CoV-2 antigen is administered to the subject as a booster regimen at least 3 months after the first vaccination is complete.

54. The method of any one of paragraphs 34-47, wherein the first encoded SARS-CoV-2 antigen is administered to the subject as a prime dose and as a boost dose in an initial vaccination and the second encoded SARS-CoV-2 antigen is administered to the subject as a boost dose more than 6 months after the initial vaccination.

55. The method of any one of paragraphs 48-52, wherein the boost dose is a seasonal boost or a pandemic shift boost to provide protection for a plurality of variants of concern.

56. The method of any one of paragraphs 50-55, wherein the boost dose is 50 μg.

57. The method of any one of paragraphs 34-56, wherein the first and/or second effective dose is 20 μg-50 μg.

58. The method of any one of paragraphs 34-57, wherein the first and/or second effective dose is 20 μg.

59. The method of any one of paragraphs 34-57, wherein the first and/or second effective dose is 25 μg.

60. The method of any one of paragraphs 34-57, wherein the first and/or second effective dose is 30 μg.

61. The method of any one of paragraphs 34-57, wherein the first and/or second effective dose is 50 μg.

62. The method of any one of paragraphs 34-57, wherein the first and/or second effective dose is 30 μg.

63. The method of any one of paragraphs 34-56, wherein the first and/or second effective dose is a 10 μg.

64. The method of any one of paragraphs 34-56, wherein the first and/or second effective dose is 10 μg-30 μg.

65. The method of any one of paragraphs 34-56, wherein the first and/or second effective dose is 10 μg-20 μg.

66. The method of any one of paragraphs 34-56, wherein the first and/or second effective dose is at least 10 μg and less than 25 μg.

67. The method of any one of paragraphs 34-56, wherein the first and/or second effective dose is 5 μg-30 μg.

68. The method of any one of paragraphs 34-56, wherein the first and/or second effective dose is at least 5 μg and less than 25 μg.

69. A method comprising administering to a subject a vaccine comprising a first nucleic acid encoding a SARS-CoV-2 antigen of a first SARS-CoV-2 virus, wherein the first SARS-CoV-2 virus is an emerging variant of a primary SARS-CoV-2 virus, wherein the antigen has an amino acid sequence with at least one amino acid mutation with respect to a corresponding protein antigen of the primary SARS-CoV-2 virus, and wherein the mutation is an amino acid substitution, deletion or insertion, and wherein the subject is seropositive for a SARS-CoV-2 antigen of the primary SARS-CoV-2 virus.

70. A method comprising administering to a subject a vaccine comprising a first nucleic acid encoding a SARS-CoV-2 antigen of a first SARS-CoV-2 virus, wherein the first SARS-CoV-2 virus is an emerging variant of a primary SARS-CoV-2 virus, wherein the antigen has an amino acid sequence with at least one amino acid mutation with respect to a corresponding protein antigen of the primary SARS-CoV-2 virus, and wherein the mutation is an amino acid substitution, deletion or insertion, and wherein the subject is seronegative for a SARS-CoV-2 antigen of the primary SARS-CoV-2 virus.

71. The method of paragraph 69 or 70 wherein the subject is administered a second dose of the vaccine between 2 weeks and 1 year after the first dose of vaccine is administered.

72. The method of paragraph 69 or 70 wherein the subject is administered a second vaccine between 2 weeks and 1 year after the vaccine is administered, wherein the second vaccine comprises a second nucleic acid encoding the corresponding protein antigen of the primary SARS-CoV-2 virus.

73. The method of paragraph 72, wherein the second vaccine comprises a mixture of the first and second nucleic acids, wherein the first nucleic acid and the second nucleic acid are present in the second vaccine at a ratio of 1:1.

74. A method comprising administering to a subject a booster vaccine comprising a nucleic acid encoding a first SARS-CoV-2 antigen from a first SARS-CoV-2 virus, wherein the subject has previously been administered at least one prime dose of a first vaccine comprising a first nucleic acid encoding the SARS-CoV-2 antigen of the first the SARS-CoV-2 virus, wherein the booster vaccine is administered in an effective amount to induce a neutralizing immune response against a second SARS-CoV-2 virus, wherein the second SARS-CoV-2 virus comprises a second SARS-CoV-2 antigen, wherein the second SARS-CoV-2 antigen has an amino acid sequence with at least one amino acid mutation with respect to a corresponding protein antigen of the first SARS-CoV-2 virus, wherein the booster vaccine is administered in a dosage of 25-100 μg at least 6 months after a first dose of the first vaccine, and wherein each of the first and second antigens are not full length stabilized spike proteins having a 2P mutation.

75. The method of paragraph 74, wherein the booster vaccine is administered in a dosage of 50 μg.

76. The method of paragraph 74 or 75, wherein the booster vaccine is administered at least about 6 months after a second dose of the first vaccine.

77. The method of paragraph 74 or 75, wherein the booster vaccine is administered 6-12 months after a second dose of the first vaccine.

78. The method of paragraph 74 or 75, wherein the booster vaccine is administered at least about 8 months after a second dose of the first vaccine.

79. The method of any one of paragraphs 74-78, wherein the boost dose is a seasonal boost or a pandemic shift boost to provide a neutralizing immune response against a plurality of variants of concern.

80. The nucleic acid of any one of paragraphs 1-18, the composition of any one of paragraphs 19-29, the mRNA of any one of paragraphs 30-33, or the method of any one of paragraphs 34-79, wherein at least one mRNA further comprises one or more non-coding sequences in an untranslated region (UTR), optionally a 5′ UTR or 3′ UTR.

81. The nucleic acid, composition, mRNA, or method of paragraph 80, wherein all of the mRNAs further comprise one or more non-coding sequences in an UTR, optionally a 5′ UTR or 3′ UTR.

82. The method or composition of paragraph 56, wherein the non-coding sequence is positioned in a 3′ UTR of an mRNA, upstream of the polyA tail of the mRNA.

83. The nucleic acid, composition, mRNA, or method of paragraph 81, wherein the non-coding sequence is positioned in a 3′ UTR of an mRNA, downstream of the polyA tail of the mRNA.

84. The nucleic acid, composition, mRNA, or method of paragraph 81, wherein the non-coding sequence is positioned in a 3′ UTR of an mRNA between the last codon of the ORF of the mRNA and the first “A” of the polyA tail of the mRNA.

85. The nucleic acid, composition, mRNA, or method of paragraph 81, wherein the non-coding sequence comprises between 1 and 10 nucleotides.

86. The nucleic acid, composition, mRNA, or method of any one of paragraphs 80-85, wherein the non-coding sequence comprises one or more RNAse cleavage sites.

87. The method or composition of paragraph 86, wherein the RNAse cleavage site comprises an RNase H cleavage site.

### EXAMPLES

### Example 1. mRNA Vaccine Induces Human Neutralizing Antibodies Against Spike Mutants from Global SARS-CoV-2 Variants

The mRNA vaccines used in the present study encode proteins containing the coronavirus SARS-CoV-2 Spike protein N-terminal domain (NTD), receptor-binding domain (RBD), and influenza hemagglutinin transmembrane (HATM) domain joined by linkers (NTD-RBD-HATM).

Both the NTD and RBD of coronavirus S proteins are known to be sites for binding of antibodies that manifest neutralizing virus activity. RBD in the case of SARS-CoV-2 is the receptor binding site of the spike protein, that binds the angiotensin-converting enzyme 2 (ACE2) as a receptor to gain entry into cells. The amino (N) terminal domain, NTD, the function of which is not thoroughly understood, seems to have a role in binding sugar moieties and in facilitating the conformational transition of the spike protein from prefusion to a post fusion conformation. See Zhou H, Chen Y, Zhang S, et al. Nat Commun. 2019; 10(1): 3068. More recently, a neutralization “supersite” has also been identified in the NTD. See McCallum, M. et al. N-terminal domain antigenic mapping reveals a site of vulnerability for SARS-CoV-2. Cell, doi:10.1016/j.cell.2021.03.028 (2021).

In experiments where a lipid nanoparticle (LNP) formulation is used, the formulation includes 0.5-15% PEG-modified lipid, 5-25% non-cationic lipid, 25-55% sterol, and 20-60% ionizable amino lipid. The PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is be cholesterol, and the ionizable amino lipid has the structure of Compound 1, for example.

### Neutralization Studies

In this study, neutralization activity of sera from clinical trial participants immunized with mRNA vaccines encoding proteins containing the N-terminal domain (NTD), receptor-binding domain (RBD), and influenza hemagglutinin transmembrane (HATM) domain joined by linkers (NTD-RBD-HATM) (“mRNA vaccine”) against recombinant vesicular stomatitis virus (VSV)-based SARS-CoV-2 in a pseudovirus neutralization (PsVN) assay is assessed with Spike protein from the USA-WA1/2020 isolate, D614G variant, the B.1.1.7 and B.1.1.351 variants, and variants that previously emerged (20E.EU1, 20A.EU2, D614G-N439K, and mink cluster 5 variant). The effect of both single mutations and combinations of mutations and deletions present in the receptor binding domain (RBD) region of the S protein is examined. Orthogonal assessments in VSV and pseudotyped lentiviral neutralization assays are also performed on sera from non-human primates (NHPs) that received the mRNA vaccine at two different doses.

To assess the ability of the mRNA vaccine to elicit potently neutralizing antibodies against the new SARS-CoV-2 variants, non-human primates (NHPs) are administered 30 μg of the mRNA vaccine twice over 3-4 weeks, and their sera is collected. Similarly, human participants are administered two 100 μg doses of the mRNA vaccine in a prime-boost regimen, and their sera is collected. The collected sera are analyzed for its neutralization properties. Neutralization activity is measured with SARS-CoV-2 full-length Spike pseudotyped recombinant VSV-AG-firefly luciferase virus, and antibody levels are measured in pseudovirus neutralization assays against homotypic SARS-CoV-2 D614, which contains the Spike protein of the USA-WA1/2020 isolate (D614), the D614G version, or Spike protein from 20A.EU1, 20A.EU2 and mink cluster 5 variants (Table 2).

NHPs are vaccinated on day 1 and 29 with 30 or 100 μg mRNA vaccines and analyzed. Neutralizing antibody responses are measured using both the lentiviral and the VSV-based PsVN assay. Pseudoviruses in both assays incorporate a full-length Spike protein, conferring the USA-WA1/2020 (D614), G614, isolated, partial, or complete set of mutations that are present in the B.1.1.7 and B.1.351 lineages.

The human sera collected as described above is further analyzed with respect to neutralization against B.1.1.7 and B.1.351.

### Methods

Animal Studies. Rhesus macaques (NHPs) are immunized with 10 or 30 μg mRNA encoding Spike protein with two proline substitutions (“mRNA vaccine”) on a prime-boost schedule, and sera is collected 4 weeks after the boosting dose (day 56).

Human Trial. Humans are immunized with 100 μg mRNA encoding proteins containing the N-terminal domain (NTD), receptor-binding domain (RBD), and influenza hemagglutinin transmembrane (HATM) domain joined by linkers (NTD-RBD-HATM) (“mRNA vaccine”) on a prime-boost schedule and sera is collected 1 week post the boost (day 36).

Lentiviral-based Pseudovirus Neutralization. To produce SARS-CoV-2 pseudotyped lentivirus, a codon-optimized CMV/R-SARS-CoV-2 S (USA-WA1/2020 isolate, GenBank: MN908947.3) plasmid is constructed and subsequently modified via site-directed mutagenesis to contain the D614G mutation. Additional Spike mutations are implemented into the D614G backbone (i.e. N501Y, E484K, N439K, and other combinations akin to those of the B.1.1.7 and B.1.351 variants; Table 3). Pseudoviruses are produced by the co-transfection of plasmids encoding a luciferase reporter, lentivirus backbone, and the SARS-CoV-2 S genes into HEK293T/17 cells (ATCC CRL-11268) as previously described (Wang et al. Evaluation of candidate vaccine approaches for MERS-CoV. Nat Commun 6, 7712). Additionally, a human transmembrane protease serine 2 (TMPRSS2) plasmid is co-transfected to produce pseudovirus (Böttcher, E. et al. Proteolytic activation of influenza viruses by serine proteases TMPRSS2 and HAT from human airway epithelium. J. Virol. 80, 9896-9898 (2006)). Neutralizing antibody responses in sera are assessed by pseudoneutralization assays as previously described (Jackson et al., 2020). Briefly, heat-inactivated serum is serially diluted in duplicate, mixed with pseudovirus, and incubated at 37° C. and 5% CO2 for roughly 45 minutes. 293T-hACE2.mF cells are diluted to a concentration of 7.5×10 4 cells/mL in DMEM (Gibco) supplemented with 10% Fetal Bovine Serum (FBS) and 1% Penicillin/Streptomycin, and added to the sera-pseudovirus mixture. Seventy-two hours later, cells are lysed and luciferase activity (in relative light units (RLU)) is measured. Percent neutralization is normalized considering uninfected cells as 100% neutralization and cells infected with pseudovirus alone as 0% neutralization. IC₅₀ titers are determined using a log(agonist) vs. normalized-response (variable slope) nonlinear regression model in Prism v8 (GraphPad).

Recombinant VS V-based Pseudovirus Neutralization. Codon-optimized full-length Spike protein of the USA-WA1/2020 isolate (D614), D614G, or the indicated Spike variants listed in Tables 2 and 3 are cloned into pCAGGS vector. To make SARS-CoV-2 full-length spike pseudotyped recombinant VSV-AG-firefly luciferase virus, BHK-21/WI-2 cells (Kerafast, EH1011) are transfected with the spike expression plasmid and subsequently infected with VSVΔG-firefly-luciferase as previously described (Whitt, 2010, Journal of Virological Methods 169, 365-374). For the neutralization assay, serially diluted serum samples are mixed with pseudovirus and incubated at 37° C. for 45 minutes. The virus-serum mix is subsequently used to infect A549-hACE2-TMPRSS2 cells for 18 hours at 37° C. before adding ONE-Glo reagent (Promega E6120) for measurement of luciferase signal (relative luminescence unit; RLU). The percentage of neutralization is calculated based on RLU of the virus only control, and subsequently analyzed using four-parameter logistic curve (Prism 8).

### Example 2. Immunization with Two Doses of mRNA Encoding a Variant Spike Protein to Generate Neutralizing Antibodies to the Variant Spike Protein

A composition comprising mRNA encoding a variant Spike protein (NTD-RBD-HATM) formulated in an LNP (as described in Example 1) is administered to subjects, such as mice, hamsters, rhesus macaques, or humans. After a period of time, such as three weeks, subjects are vaccinated with the same composition, as a booster dose. The variant Spike proteins encoded by the mRNAs are described in Table 3. Spike protein-specific neutralization titers are quantified by a VSVΔG-based SARS-CoV-2 pseudovirus neutralization assay in which the VSVΔG-based SARS-CoV-2 pseudovirus expresses the same variant Spike protein encoded by the mRNA in the composition.

After a period of time, such as three weeks after the booster dose, protection from infection is evaluated by introducing SARS-CoV-2 expressing wild-type or variant Spike protein to the nasal passages of a subject. Subjects are then monitored over the course of a period of time, such as two weeks, to quantify the amount of SARS-CoV-2 RNA or protein present in the nasal passages over the duration of the monitoring period following viral challenge.

TABLE 3
Variant Spike proteins encoded by mRNAs described in Examples 2-5.

| Variant Spike Protein | Mutations relative to wild-type Spike protein |
|---|---|
| NTD-RBD-HATM 1 | E484K |
| NTD-RBD-HATM 2 | K417N E484K N501Y |
| NTD-RBD-HATM 3 | L18F D80A D215G L242 Δ244 R246I K417N |
| | E484K N501Y |
| NTD-RBD-HATM 4 | ΔH69 ΔV70 ΔY144 N501Y |
| NTD-RBD-HATM 5 | L18F ΔH69 ΔV70 D80A ΔY144 D215G L242 |
| | Δ244 R246I K417N E484K N501Y |

### Example 3. Immunization with a Prime Dose of mRNA Encoding Wild-Type Spike Protein and a Booster Dose of mRNA Encoding a Variant Spike Protein to Generate Neutralizing Antibodies to the Variant Spike Protein

A composition comprising mRNA encoding a Spike protein antigen fusion (NTD-RBD-HATM) formulated in an LNP (as described in Example 1) is administered to subjects, such as mice, hamsters, rhesus macaques, or humans. After a period of time, such as three weeks, subjects are vaccinated with a composition comprising mRNA encoding a variant Spike protein (NTD-RBD-HATM) formulated in an LNP, as a booster dose. The variant Spike proteins encoded by the mRNAs are described in Table 3. Spike protein-specific titers are quantified by a VSVΔG-based SARS-CoV-2 pseudovirus neutralization assay in which the VSVΔG-based SARS-CoV-2 pseudovirus expresses the same variant Spike protein encoded by the mRNA in the composition used in the booster dose.

After a period of time, such as three weeks after the booster dose, protection from infection is evaluated by introducing SARS-CoV-2 expressing wild-type or variant Spike protein to the nasal passages of a subject. Subjects are then monitored over the course of a period of time, such as two weeks, to quantify the amount of SARS-CoV-2 RNA or protein present in the nasal passages over the duration of the monitoring period following viral challenge.

### Example 4. Immunization with a Prime Dose of mRNA Encoding Wild-Type Spike Protein and a Booster Dose of Multiple mRNAs Encoding Variant Spike Proteins to Generate Neutralizing Antibodies to the Variant Spike Proteins

A composition comprising mRNA encoding a Spike protein antigen fusion (NTD-RBD-HATM) formulated in an LNP (as described in Example 1) is administered to subjects, such as mice, hamsters, rhesus macaques, or humans. After a period of time, such as three weeks, subjects are vaccinated with a composition comprising multiple mRNAs, each encoding a different variant Spike protein antigen fusion (NTD-RBD-HATM), formulated in an LNP, as a booster dose. The variant Spike proteins antigen fusions are encoded by the mRNAs are described in Table 3. Spike protein-specific titers are quantified by a VSVΔG-based SARS-CoV-2 pseudovirus neutralization assay in which the VSVΔG-based SARS-CoV-2 pseudovirus expresses wild-type sequences in the Spike protein antigen fusion proteins or a variant Spike protein antigen fusion encoded by the composition in the booster dose.

After a period of time, such as three weeks after the booster dose, protection from infection is evaluated by introducing SARS-CoV-2 expressing wild-type or variant Spike protein to the nasal passages of a subject. Subjects are then monitored over the course of a period of time, such as two weeks, to quantify the amount of SARS-CoV-2 RNA or protein present in the nasal passages over the duration of the monitoring period following viral challenge.

### Example 5. Immunization with Two Doses of Multiple mRNAs Encoding Variant Spike Proteins to Generate Neutralizing Antibodies to the Variant Spike Proteins

A composition comprising multiple mRNAs, each encoding a different variant Spike protein antigen fusion (NTD-RBD-HATM), formulated in an LNP (as described in Example 1) is administered to subjects, such as mice, hamsters, rhesus macaques, or humans. The variant Spike protein antigen fusion encoded by the mRNAs are described in Table 3. After a period of time, such as three weeks, subjects are vaccinated with the same composition, as a first booster dose. Spike protein-specific titers are quantified by a VSVΔG-based SARS-CoV-2 pseudovirus neutralization assay in which the VSVΔG-based SARS-CoV-2 pseudovirus expresses wild-type Spike protein or a variant Spike protein encoded by the composition.

After a period of time, such as three weeks after the first booster dose, protection from infection is evaluated by introducing SARS-CoV-2 expressing wild-type or variant Spike protein to the nasal passages of a subject. Subjects are then monitored over the course of a period of time, such as two weeks, to quantify the amount of SARS-CoV-2 RNA or protein present in the nasal passages over the duration of the monitoring period following viral challenge. In certain populations, a third dose may be administered as another booster dose.

### Example 6: Coronavirus Strain Challenge

The instant study is designed to test the efficacy in hamsters, mice and/or rabbits of candidate coronavirus vaccines comprising an mRNA as disclosed herein encoding a coronavirus antigen (e.g., the spike (S) protein, the S1 subunit (S1) of the spike protein, or the S2 subunit (S2) of the spike protein, a domain etc.), such as a SARS-CoV-2 antigen, against a lethal challenge with a coronavirus. Animals are challenged with a lethal dose (10×LD90; ˜100 plaque-forming units; PFU) of coronavirus.

The animals used are ˜6-8 week old animals in groups of ˜10. Animals are vaccinated on weeks 0 and 3 via an IM, ID or IV route of administration. Candidate vaccines are chemically modified or unmodified. Animal serum is tested for microneutralization. Animals are then challenged with ˜1 LD90 of coronavirus on week ˜6-8 via an IN, IM, ID or IV route of administration. Endpoint is day ˜13-15 post infection, death or euthanasia. Animals displaying severe illness as determined by >30% weight loss, extreme lethargy or paralysis are euthanized. Body temperature and weight are assessed and recorded daily.

### Example 7—In Vivo Expression of SARS-CoV-2 mRNA Vaccine Constructs

BALB/c mice, 6-8 weeks of age, are administered either 2 μg or 10 μg of a COVID-19 construct or Tris buffer (as a control) intramuscularly in each hind leg. The constructs comprise any of the mRNA encoding antigens disclosed herein in cationic lipid nanoparticles, 10.7 mM sodium acetate, 8.7% sucrose, 20 mM Tris (pH 7.5). One day later, spleens and lymph nodes are collected to detect protein expression using flow cytometry.

### Example 8—SARS-CoV-2 mRNA Protects Humanized Mice from Lethal Challenge

Humanized DPP4 288/330^{+/+} mice are immunized at weeks 0 and 3 weeks with 0.01, 0.1, or 1 μg of SARS-CoV-2 mRNA encoding antigens. Mock-immunized mice are immunized with PBS. Four weeks post-boost, mice are challenged with a lethal dose of mouse-adapted SARS-CoV. Following challenge, mice are monitored for weight loss and signs of viral infection. At days 3 and 5 post-challenge, lungs from 5 mice/group are harvested for analysis of viral titers and hemorrhage.

### Example 9—Immunogenicity of SARS-CoV-2 mRNA Encoding the B.1.351 Spike Protein Antigen (1^{st} and 2^{nd} Booster Doses)

BALB/c mice were immunized at days 1 and 22 with 0.1 μg or 1 μg of an mRNA encoding a Spike protein antigen fusion (NTD-RBD-HATM) (n=8/group). The mice were subsequently administered a first booster dose (dose 3; 0.1 μg or 1 μg) on day 213 and a second booster dose (dose 4; 0.1 μg or 1 μg) on day 234. The first and second booster doses comprise mRNA encoding the Spike protein antigen fusion having the following mutations relative to the wild-type SARS-CoV-2 virus: L18F, ΔH69, ΔV70, D80A, ΔY144, D215G, L242, A244, R246I, K417N, E484K, and N501Y). In each vaccine, the mRNA was formulated in lipid nanoparticles (LNPs) including 0.5-15% PEG-modified lipid, 5-25% non-cationic lipid, 25-55% sterol, and 20-60% ionizable amino lipid. The PEG-modified lipid was 1,2 dimyristoyl-sn-glycerol, methoxypolyethyleneglycol (PEG2000 DMG), the non-cationic lipid was 1,2 distearoyl-sn-glycero-3-phosphocholine (DSPC), the sterol was cholesterol, and the ionizable amino lipid had the structure of Compound 1, for example.

Blood was collected on days 36, 212, and 233, and analyzed by pseudovirus neutralization assays as described herein. Briefly, pseudoviruses were constructed with Wuhan-Hu-1 Spike with mutation D614G (comparator variant, known as D614G) alone or combined with the additional mutations found in B.1.351 (L18F, D80A, D215G, A242-244, R246I, K417N, E484K, N501Y, A701V). Neutralization assays were performed using a validated lentivirus-based Spike-pseudotyped virus assay in 293T cells stably transduced to overexpress ACE24.

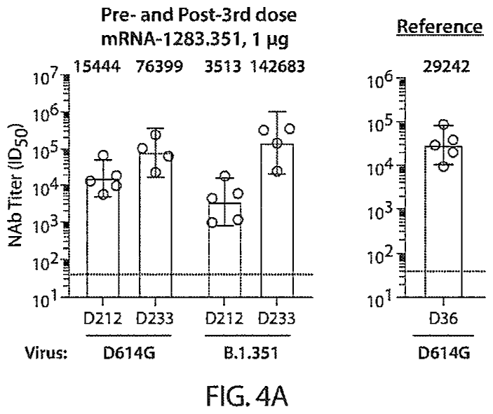

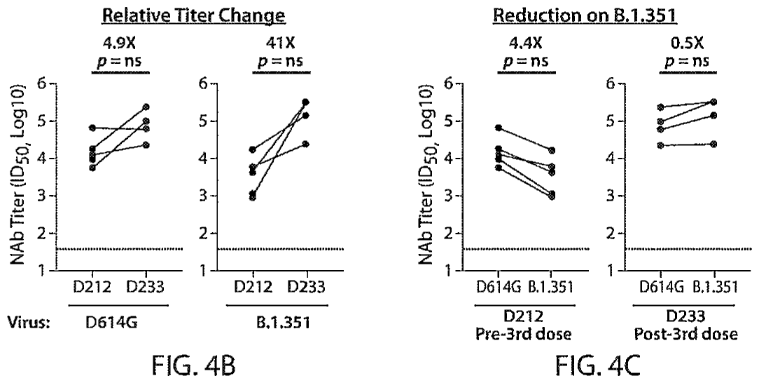

FIG. 4A-4C show neutralizing antibody titers in mice on day 212 (before the third dose, 1 μg) and day 233 (after the third dose). As shown in FIG. 4A, the neutralizing antibody titer was increased from day 212 to day 233 against the D614 variant Spike protein and more dramatically against the B.1.351 variant Spike protein. In both instances, the antibody titers on day 233 dropped about twofold over the six-month period between day 36 and day 212. For example, the neutralizing antibody titer (ID₅₀) against D614G was 29242 at day 36 and 15,444 at day 212 (data not shown). As shown in FIG. 4B, the neutralization titer on day 233 was 4.9× higher than on day 212 against the D614G variant Spike protein, and 41× higher against the B.1.351 variant Spike protein. FIG. 4C shows the D614G variant Spike protein elicited neutralization titers 4.4× higher than those against the B.1.351 variant Spike protein; however, after the third dose (day 233), the neutralization titers elicited against the D614G variant Spike protein were 0.5× those elicited by the B.1.351 variant Spike protein.

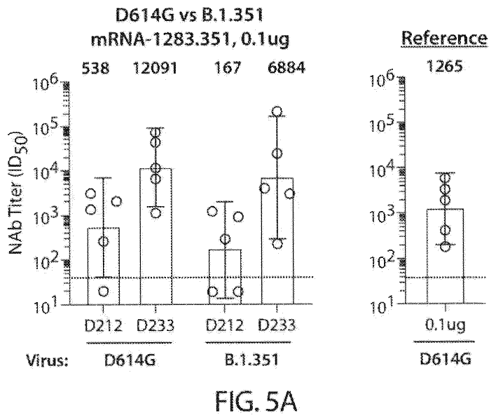

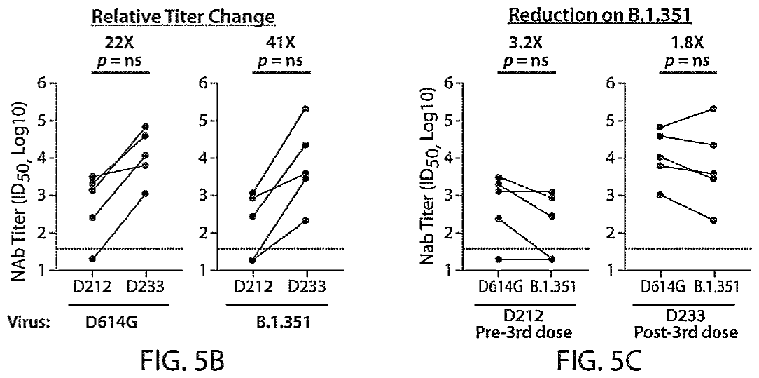

FIG. 5A-5C show neutralizing antibody titers in mice on day 212 (before the third dose, 0.1 μg) and day 233 (after the third dose). As shown in FIG. 5A, the neutralizing antibody titer was increased from day 212 to day 233 against the D614 variant Spike protein and against the B.1.351 variant Spike protein. In both instances, the antibody titers on day 233 were greater than those measured at day 36 (14 days after the second dose). As shown in FIG. 5B, the neutralization titer on day 233 was 22× higher than on day 212 against the D614G variant Spike protein, and 41× higher against the B.1.351 variant Spike protein. FIG. 5C shows the D614G variant Spike protein elicited neutralization titers 3.2× higher than those against the B.1.351 variant Spike protein; however, after the third dose (day 233), the neutralization titers elicited against the D614G variant Spike protein were 1.8× higher than those elicited by the B.1.351 variant Spike protein.

### Example 10. Immunization with Two Doses of Multiple mRNAs Encoding Variant Spike Proteins to Generate Neutralizing Antibodies to the Variant Spike Proteins

BALB/c mice, 6-8 weeks of age, are administered either 1 μg or 10 μg of mRNA encoding a SARS-CoV-2 antigen or PBS (as a control) intramuscularly in one hind leg (formulated as a 50 μL dose) on day 1 and day 22. In each vaccine, the mRNA is formulated in lipid nanoparticles (LNPs) including 0.5-15% PEG-modified lipid, 5-25% non-cationic lipid, 25-55% sterol, and 20-60% ionizable amino lipid. The PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol, and the ionizable amino lipid has the structure of Compound 1, for example. The groups tested are shown in Table 4.

TABLE 4
Experimental Groups for Example 10 and 12.

| N= | mRNA | Dose |
|---|---|---|
| 10 | PBS | |
| 10 | NTD-RBD-HATM | 10 |
| 10 | | 1 |
| 10 | NTD-RBD-HATM_E484K | 10 |
| 10 | | 1 |
| 10 | NTD-RBD-HATM_K417N_E484K_N501Y | 10 |
| 10 | | 1 |
| 10 | NTD-RBD- | 10 |
| 10 | HATM_L18F_D80A_D215G_L242_244del_R246I_K417N_E484K_N501Y | 1 |
| 10 | NTD-RBD-HATM_H69del_V70del_Y144del_N501Y | 10 |
| 10 | | 1 |
| 10 | 1:1 mix of NTD-RBD-HATM & NTD-RBD- | 10 (5 + 5) |
| 10 | HATM_K417N_E484K_N501Y | 1(0.5 + 0.5) |
| 10 | 1:1 mix of NTD-RBD-HATM & NTD-RBD-HATM_(RSA) | 10 (5 + 5) |
| 10 | L18F_D80A_D215G_L242_244del_R246I_K417N_E484K_N501Y | 1 (0.5 + 0.5) |

Blood samples are taken from the mice on day 14, day 21, and day 36 and analyzed by ELISA and neutralization assays as described herein.

### Example 11: In Vitro Neutralization Screening

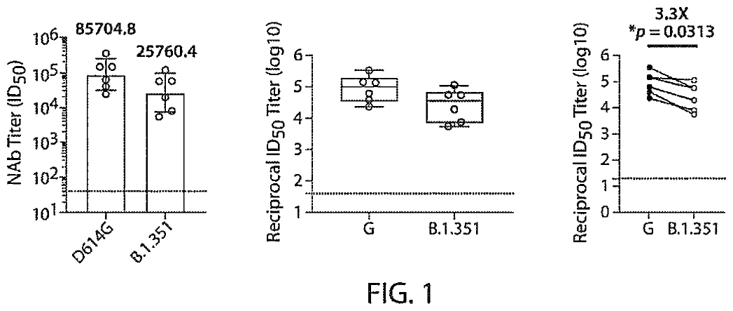

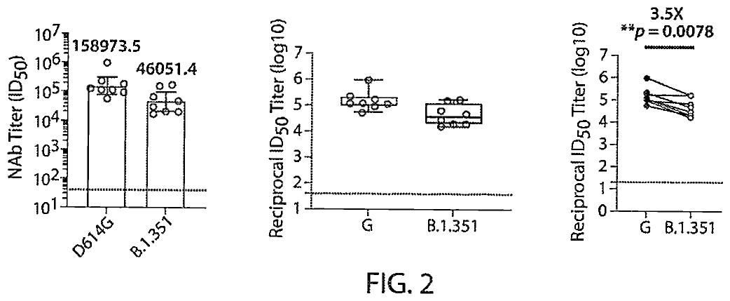

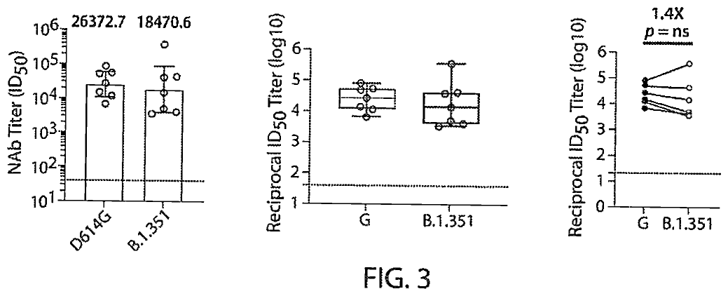

Codon-optimized full-length spike protein of the D614G variant or the spike protein of the B.1.351 variant were cloned into a pCAGGS vector. For the neutralization assay, serially diluted serum samples were mixed with pseudovirus and incubated at 37° C. for 45 minutes. The serum samples were from mice that had been administered 1 μg of NTD-RBD-HATM, NTD_ext-RBD_ext-TM or RBD_WH2020_NatSP_317_516_TM (RBD-HATM). The virus-serum mix was subsequently used to infect A549-hACE2-TMPRSS2 cells for 18 hours at 37° C. before measuring neutralizing antibody titers. The results are shown in FIGS. 1-3 and demonstrate that NTD-RBD-HATM had a 3.3-fold decrease in neutralization titer against the B.1.351 variant compared to the titer resulting from the D614G pseudovirus, NTD_ext-RBD_ext-TM resulted in a 3.5-fold decrease in neutralization titer between the D614 variant and the B.1.351 variant, and RBD_WH2020_NatSP_317_516_TM did not have a significant decrease in neutralization titer against the B.1.351 variant compared to the titer resulting from the D614G pseudovirus.

### Example 12: Immunization with Four Doses of mRNAs Encoding Variant Spike Proteins to Generate Neutralizing Antibodies to Variant Spike Proteins

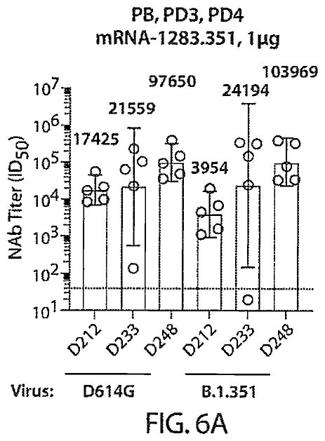

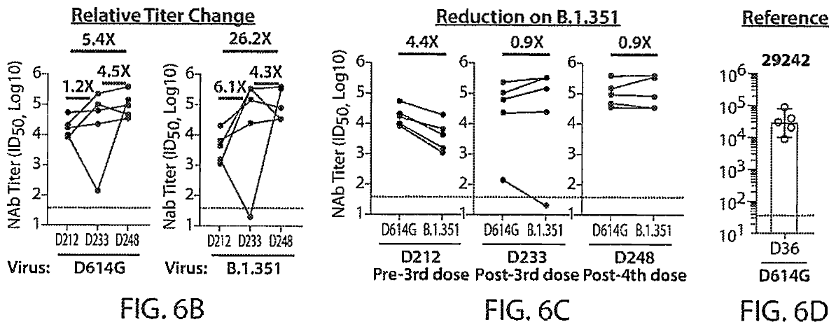

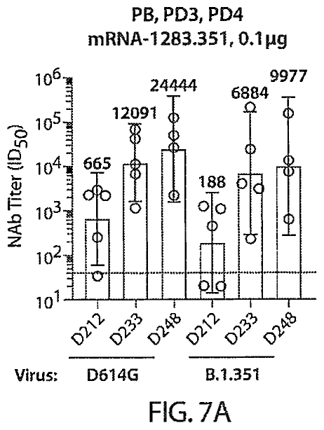

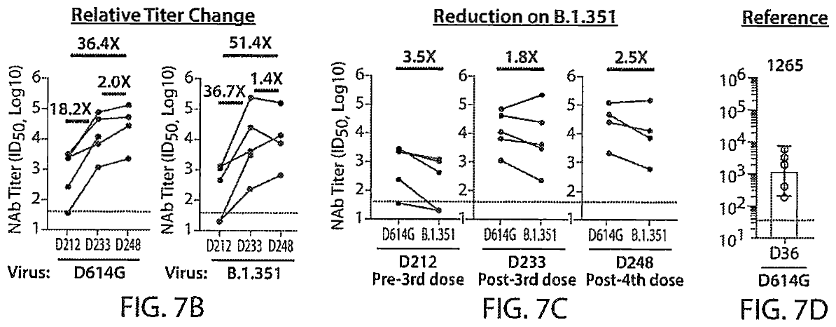

BALB/c mice, 6-8 weeks of age, were administered either 1 μg (FIGS. 6A-6D) or 0.1 μg (FIGS. 7A-7D) of mRNA encoding a SARS-CoV-2 antigen, specifically NTD-RBD-HATM, or PBS (as a control) intramuscularly in one hind leg (formulated as a 50 μL dose) on day 1 (1^{st} dose, prime) and day 22 (2^{nd} dose, booster). At days 213 (3^{rd} dose) and 234 (4^{th} dose), mice were administered either 1 μg (FIGS. 6A-6D) or 0.1 μg (FIGS. 7A-7D) of mRNA encoding a variant SARS-CoV-2 antigen (mRNA-1283.351), NTD-RBD-HATM_L18F_D80A_D215G_L242_244del_R246I_K417N_E484K_N501Y, which contained the mutations associated with the B.1.351 variant. In each vaccine, the mRNA was formulated in lipid nanoparticles (LNPs) including 0.5-15% PEG-modified lipid, 5-25% non-cationic lipid, 25-55% sterol, and 20-60% ionizable amino lipid. The PEG-modified lipid was 1,2 dimyristoyl-sn-glycerol, methoxypolyethyleneglycol (PEG2000 DMG), was non-cationic lipid is 1,2 distearoyl-sn-glycero-3-phosphocholine (DSPC), the sterol was cholesterol, and the ionizable amino lipid had the structure of Compound 1, for example. Sera were collected from mice on days 212 (prior to 3^{rd} dose), 233 (21 days after 3^{rd} dose, prior to 4^{th} dose), and 248 (14 days after 4^{th} dose), and analyzed for neutralization titers against VSV-based pseudoviruses expressing a SARS-CoV-2 Spike protein comprising either 1) a D614G mutation relative to the sequence of the WH2020 full-length Spike protein, or 2) mutations associated with the B.1.351 variant.

For mice administered 1 μg doses of mRNA in each vaccine, the results of these neutralization assays are shown in FIGS. 6A-6D. Each of the 3^{rd} and 4^{th} doses increased mean neutralization titers of sera against the D614G and B.1.351 Spike proteins (FIG. 6A). Neutralization titers towards pseudoviruses carrying each Spike protein increased over time, with this increase being more pronounced in neutralization titers towards the B.1.351 Spike protein (FIG. 6B). Prior to the 3^{rd} dose, sera were approximately 4.4 times as effective at neutralizing the D614G Spike protein than the B.1.351 Spike protein (FIG. 6C). However, after each of the 3^{rd} and 4^{th} doses, neutralization titers were approximately equivalent between both Spike proteins, with sera neutralizing the D614G Spike protein approximately 90% as well as the B.1.351 Spike protein (FIG. 6C). Additionally, neutralization titers towards either Spike protein after the 4^{th} dose were over 3-fold greater than reference neutralization titers towards the D614G Spike protein at day 36, 14 days after the 2^{nd} dose (FIGS. 6A, 6D). These results indicate that the administration of mRNAs encoding variant Spike proteins effectively elicit neutralizing antibodies towards the variant Spike protein, and boost the antibody response to Spike proteins encoded by previously administered initial mRNAs.

For mice administered 0.1 μg doses of mRNA in each vaccine, the results of these neutralization assays are shown in FIGS. 7A-7D. Each of the 3^{rd} and 4^{th} doses increased mean neutralization titers of sera against the D614G and B.1.351 Spike proteins (FIG. 7A). Neutralization titers towards each Spike protein increased over time, with this increase being more pronounced in neutralization titers towards pseudoviruses carrying the B.1.351 Spike protein (FIG. 7B). Prior to the 3^{rd} dose, sera were approximately 3.5 times as effective at neutralizing the D614G Spike protein than the B.1.351 Spike protein (FIG. 7C). However, after each of the 3^{rd} and 4^{th} doses, neutralization titers were greater towards pseudoviruses carrying the B.1.351 Spike protein, with sera neutralizing pseudoviruses carrying the B.1.351 Spike protein approximately 1.8 times as well as the D614G Spike protein after the 3^{rd} dose, and 2.5 times as well after the 4^{th} dose (FIG. 7C). Additionally, neutralization titers towards pseudoviruses carrying either Spike protein after the 4^{th} dose were over 8-fold greater than reference neutralization titers towards the D614G Spike protein at day 36, 14 days after the 2n d dose (FIGS. 7A, 7D). These results indicate that the administration of mRNAs encoding variant Spike proteins effectively elicit neutralizing antibodies towards pseudoviruses carrying the variant Spike protein, and boost the antibody response specific to Spike proteins encoded by previously administered mRNAs.

### Example 13: Immunization with Two Doses of mRNA Encoding a NTD-ext-RBD-ext-TM

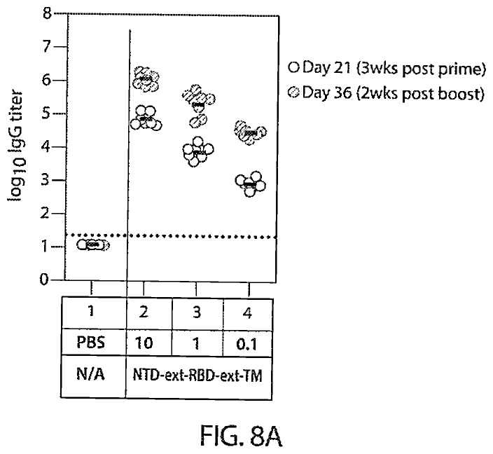

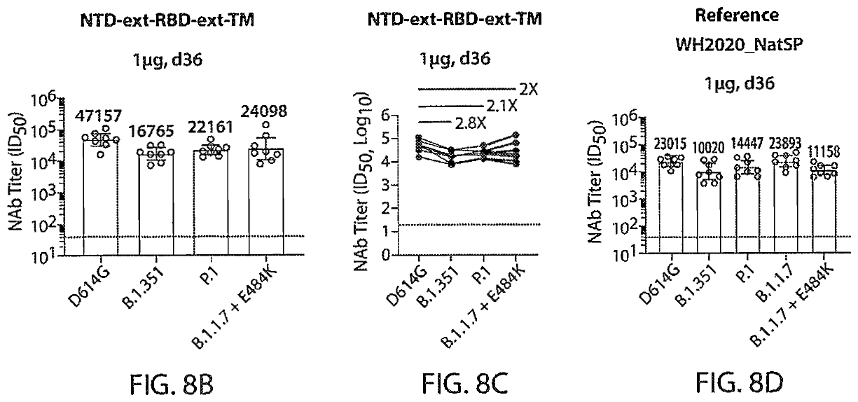

BALB/c mice, 6-8 weeks of age, were administered 0.1 μg, 1 μg, or 10 μg of mRNA encoding a SARS-CoV-2 antigen, specifically NTD_ext-RBD_ext-TM (NTD-ext-RBD-ext-TM), or PBS (as a control) intramuscularly in one hind leg (formulated as a 50 μL dose) on day 1 (1^{st} dose, prime) and day 22 (2^{nd} dose, booster). Sera were collected from mice at days 21 (3 weeks after 1^{st} dose, before 2^{nd} dose), and 36 (2 weeks after 2^{nd} dose), and tested by ELISA to quantify total IgG specific to a parental SARS-CoV-2 Spike protein with the WH2020 amino acid sequence. Each 1^{st} dose of mRNA encoding NTD-ext-RBD-ext-TM Spike antigen elicited a robust SARS-CoV-2 Spike protein-specific antibody response, with the 2^{nd} dose boosting IgG titers by 10- to 100-fold in each dose group (FIG. 8A).

Sera obtained at day 36 from mice vaccinated with two 1 μg doses of mRNA encoding NTD-ext-RBD-TM were also evaluated for neutralization activity against a panel of VSV-based pseudoviruses, each expressing a different SARS-CoV-2 Spike protein. The panel of Spike proteins tested is shown in Table 5, and included a D614G Spike protein, a B.1.351 Spike protein, a P.1 Spike protein, a B.1.1.7 Spike protein, and a B.1.1.7 Spike protein comprising an E484K mutation. The results of these neutralization assays are shown in FIGS. 8B-8D. Two 1 μg doses of mRNA encoding NTD-ext-RBD-ext-TM elicited robust neutralizing antibody responses towards pseudoviruses carrying each of the Spike proteins tested (FIG. 8B). Neutralization titers towards each of the variant Spike proteins, B.1.351, P.1, and B.1.1.7 with E484K mutation, were 2- to 3-fold lower than neutralization titers towards the D614G reference Spike protein (FIG. 8C). However, compared to reference sera obtained from mice administered two 1 μg doses of mRNA encoding 2P-stabilized WH2020 full-length Spike protein, sera from mice administered two 1 μg doses of mRNA encoding NTD-ext-RBD-ext-TM were 1.5- to 2 times more effective at neutralizing pseudoviruses carrying each of the Spike proteins tested (FIGS. 8B, 8D). These results indicate that mRNA encoding an NTD-RBD-TM fusion protein with extended receptor-binding and transmembrane domains elicits robust neutralizing antibody responses to multiple SARS-CoV-2 variant Spike proteins.

TABLE 4
Spike proteins tested in neutralization assays

| | Neutralization titer elicited | Neutralization |
|---|---|---|
| | by 1 μg NTD-ext-RBD-ext- | titer elicited |
| Spike protein | TM (mean ID₅₀) | by PBS (ID₅₀) |
| D614G | 47517 | 20 (below L.O.D.) |
| B.1.351 | 16765 | 20 (below L.O.D.) |
| P.1 | 22161 | 20 (below L.O.D.) |
| B.1.1.7 + E484K | 24098 | — |

### Example 14— SARS-CoV-2 mRNA Third Dose Immunogenicity

BALB/c mice, 6-8 weeks of age, were administered 1 μg of mRNA encoding a SARS-CoV-2 antigen or PBS (as a control) intramuscularly in one hind leg (formulated as a 50 μL dose) on day 1 and day 22. The mice were then administered a third dose at week 8 (day 57), comprising mRNA encoding a spike protein variant (mRNA-1283, mRNA-1283.351, mRNA-1283.617.2, mRNA-1283+mRNA-1283.351, mRNA-1283+mRNA-1283.617.2, or mRNA-1283+mRNA-1283.351+mRNA-1283.617.2). In each vaccine, the mRNA was formulated in lipid nanoparticles (LNPs) including 0.5-15% PEG-modified lipid, 5-25% non-cationic lipid, 25-55% sterol, and 20-60% ionizable amino lipid. The PEG-modified lipid was 1,2 dimyristoyl-sn-glycerol, methoxypolyethyleneglycol (PEG2000 DMG), the non-cationic lipid was 1,2 distearoyl-sn-glycero-3-phosphocholine (DSPC), the sterol was cholesterol, and the ionizable amino lipid had the structure of Compound 1, for example.

Blood samples were taken on days 21, 36, and 78 and analyzed by ELISA and neutralization assays as described herein. The neutralizing titers are shown in Table 5.

TABLE 5
Neutralization Titers (Geometric Mean Titer, GMT)

| | | S2P | | | NTD | RBD |
|---|---|---|---|---|---|---|
| | | Day | Day | Day | Day | Day |
| Formulation/ | | 21- | 36- | 78- | 78- | 78- |
| Material | Dose(ug) | GMT | GMT | GMT | GMT | GMT |
| PBS | | 13 | 13 | 13 | 13 | 13 |
| PBS | | 13 | 13 | 13 | 13 | 13 |
| PBS | | 13 | 13 | 13 | 13 | 13 |
| mRNA-1283 | 1 | 6179 | 135176 | 424079 | 79454 | 210261 |
| mRNA-1283.351 | 1 | 11888 | 100389 | 451348 | 17425 | 49441 |
| mRNA- | 1 | 5287 | 130957 | 504622 | 47021 | 92327 |
| 1283.617.2 | | | | | | |
| mRNA-1283 + | 1 | 5055 | 141217 | 429434 | 30676 | 69192 |
| 1283.351 | | | | | | |
| mRNA-1283 + | 1 | 4763 | 115615 | 593690 | 67365 | 139108 |
| 1283.617.2 | | | | | | |
| mRNA- | 1 | 5398 | 98220 | 516556 | 41462 | 133570 |
| 1283 + 1283.351 + | | | | | | |
| 1283.617.2 | | | | | | |

As is shown above, all third (booster) doses resulted in increased neutralizing antibody production above that observed at day 21 (just before administration of the second dose) and at day 36 (two weeks following administration of the second dose). For the samples challenged with the S2P antigen (spike protein comprising a double proline mutation), there was an increase in titer levels for all the groups after the third shot (booster). All of the titers were within 2-fold of on another, indicating that the combination formulations worked comparably to the single (monovalent) formulations. Among the monovalent groups, the highest titers were achieved with mRNA-1283.617.2, while the mRNA-1283+1283.617.2 formulation achieved the highest titers. With respect to the NTD antigen and the RBD antigen, the mRNA-1283 formulation resulted in the greatest antibody titers.

### REFERENCES

1 Zhou, P. et al. A pneumonia outbreak associated with a new coronavirus of probable bat origin. Nature 579, 270-273, doi:10.1038/s41586-020-2012-7 (2020).
2 Coronaviridae Study Group of the International Committee on Taxonomy of, V. The species Severe acute respiratory syndrome-related coronavirus: classifying 2019-nCoV and naming it SARS-CoV-2. Nat Microbiol 5, 536-544, doi:10.1038/s41564-020-0695-z (2020).
3 Wrapp, D. et al. Cryo-EM structure of the 2019-nCoV spike in the prefusion conformation. Science 367, 1260-1263, doi:10.1126/science.abb2507 (2020).
4 Jackson, L. A. et al. An mRNA Vaccine against SARS-CoV-2—Preliminary Report. N Engl J Med 383, 1920-1931, doi:10.1056/NEJMoa2022483 (2020).
5 Baden, L. R. et al. Safety and immunogenicity of two heterologous HIV vaccine regimens in healthy, HIV-uninfected adults (TRAVERSE): a randomised, parallel-group, placebo-controlled, double-blind, phase 1/2a study. Lancet HIV 7, e688-e698, doi:10.1016/52352-3018(20)30229-0 (2020).
6 FDA (2021) COVID-19 Vaccines. https://www.fda.gov/emergency-preparedness-and-response/coronavirus-disease-2019-covid-19/covid-19-vaccines.
7 Korber, B. et al. Tracking Changes in SARS-CoV-2 Spike: Evidence that D614G Increases Infectivity of the COVID-19 Virus. Cell 182, 812-827 e819, doi:10.1016/j.cell.2020.06.043 (2020).
8 Plante, J. A. et al. Spike mutation D614G alters SARS-CoV-2 fitness. Nature 592, 116-121, doi:10.1038/s41586-020-2895-3 (2021).
9 Volz, E. et al. Evaluating the Effects of SARS-CoV-2 Spike Mutation D614G on Transmissibility and Pathogenicity. Cell 184, 64-75 e11, doi:10.1016/j.cell.2020.11.020 (2021).
10 Yurkovetskiy, L. et al. Structural and Functional Analysis of the D614G SARS-CoV-2 Spike Protein Variant. Cell 183, 739-751 e738, doi:10.1016/j.cell.2020.09.032 (2020).
11 Walls, A. C. et al. Structure, Function, and Antigenicity of the SARS-CoV-2 Spike Glycoprotein. Cell 181, 281-292 e286, doi:10.1016/j.cell.2020.02.058 (2020).
12 Letko, M., Marzi, A. & Munster, V. Functional assessment of cell entry and receptor usage for SARS-CoV-2 and other lineage B betacoronaviruses. Nat Microbiol 5, 562-569, doi:10.1038/s41564-020-0688-y (2020).
13 Wang, Q. et al. Structural and Functional Basis of SARS-CoV-2 Entry by Using Human ACE2. Cell 181, 894-904 e899, doi:10.1016/j.cell.2020.03.045 (2020).
14 Shang, J. et al. Cell entry mechanisms of SARS-CoV-2. Proc Natl Acad Sci USA 117, 11727-11734, doi:10.1073/pnas.2003138117 (2020).
15 Keech, C. et al. Phase 1-2 Trial of a SARS-CoV-2 Recombinant Spike Protein Nanoparticle Vaccine. N Engl J Med 383, 2320-2332, doi:10.1056/NEJMoa2026920 (2020).
16 Walsh, E. E. et al. Safety and Immunogenicity of Two RNA-Based Covid-19 Vaccine Candidates. N Engl J Med 383, 2439-2450, doi:10.1056/NEJMoa2027906 (2020).
17 Anderson, E. J. et al. Safety and Immunogenicity of SARS-CoV-2 mRNA-1273 Vaccine in Older Adults. N Engl J Med 383, 2427-2438, doi:10.1056/NEJMoa2028436 (2020).
18 McCallum, M. et al. N-terminal domain antigenic mapping reveals a site of vulnerability for SARS-CoV-2. Cell, doi:10.1016/j.cell.2021.03.028 (2021).
19 Li, Q. et al. The Impact of Mutations in SARS-CoV-2 Spike on Viral Infectivity and Antigenicity. Cell 182, 1284-1294 e1289, doi:10.1016/j.cell.2020.07.012 (2020).
20 Tegally, H. et al. Detection of a SARS-CoV-2 variant of concern in South Africa. Nature, doi:10.1038/s41586-021-03402-9 (2021).
21 Shen, X. et al. SARS-CoV-2 variant B.1.1.7 is susceptible to neutralizing antibodies elicited by ancestral spike vaccines. Cell Host Microbe, doi:10.1016/j.chom.2021.03.002 (2021).
22 Wibmer, C. K. et al. SARS-CoV-2 501Y.V2 escapes neutralization by South African COVID-19 donor plasma. Nat Med, doi:10.1038/s41591-021-01285-x (2021).
23 Voysey, M. et al. Safety and efficacy of the ChAdOx1 nCoV-19 vaccine (AZD1222) against SARS-CoV-2: an interim analysis of four randomised controlled trials in Brazil, South Africa, and the UK. Lancet 397, 99-111, doi:10.1016/50140-6736(20)32661-1 (2021).
24 Voysey, M. et al. Single-dose administration and the influence of the timing of the booster dose on immunogenicity and efficacy of ChAdOx1 nCoV-19 (AZD1222) vaccine: a pooled analysis of four randomised trials. Lancet 397, 881-891, doi:10.1016/50140-6736(21)00432-3 (2021).
25 Andreano, E. et al. SARS-CoV-2 escape in vitro from a highly neutralizing COVID-19 convalescent plasma. bioRxiv, doi:10.1101/2020.12.28.424451 (2020).
26 Hoffmann, M. et al. SARS-CoV-2 variants B.1.351 and P.1 escape from neutralizing antibodies. Cell, doi:10.1016/j.cell.2021.03.036 (2021).
27 Garcia-Beltran, W. F. et al. Multiple SARS-CoV-2 variants escape neutralization by vaccine-induced humoral immunity. Cell, doi:10.1016/j.cell.2021.03.013 (2021).
28 Starr, T. N. et al. Deep Mutational Scanning of SARS-CoV-2 Receptor Binding Domain Reveals Constraints on Folding and ACE2 Binding. Cell 182, 1295-1310 e1220, doi:10.1016/j.cell.2020.08.012 (2020).
29 Starr, T. N. et al. Deep mutational scanning of SARS-CoV-2 receptor binding domain reveals constraints on folding and ACE2 binding. bioRxiv, doi:10.1101/2020.06.17.157982 (2020).
30 Tada, T. et al. Decreased neutralization of SARS-CoV-2 global variants by therapeutic anti-spike protein monoclonal antibodies. bioRxiv, doi:10.1101/2021.02.18.431897 (2021).
31 Tada, T. et al. Neutralization of viruses with European, South African, and United States SARS-CoV-2 variant spike proteins by convalescent sera and BNT162b2 mRNA vaccine-elicited antibodies. bioRxiv, doi:10.1101/2021.02.05.430003 (2021).
32 Deng, X. et al. Transmission, infectivity, and antibody neutralization of an emerging SARS-CoV-2 variant in California carrying a L452R spike protein mutation. medRxiv, doi:10.1101/2021.03.07.21252647 (2021).
33 Greaney, A. J. et al. Comprehensive mapping of mutations in the SARS-CoV-2 receptor-binding domain that affect recognition by polyclonal human plasma antibodies. Cell Host Microbe 29, 463-476 e466, doi:10.1016/j.chom.2021.02.003 (2021).
34 Wang, P. et al. Antibody resistance of SARS-CoV-2 variants B.1.351 and B.1.1.7. Nature, doi:10.1038/s41586-021-03398-2 (2021).
35 Zhou, H. et al. B.1.526 SARS-CoV-2 variants identified in New York City are neutralized by vaccine-elicited and therapeutic monoclonal antibodies. bioRxiv, doi:10.1101/2021.03.24.436620 (2021).
36 Wu, K. et al. Serum Neutralizing Activity Elicited by mRNA-1273 Vaccine. N Engl J Med, doi:10.1056/NEJMc2102179 (2021).
37 Novavax (2021). Novavax COVID-19 Vaccine Demonstrates 89.3% Efficacy in UK Phase 3 Trial. Press Release Jan. 28, 2021: https://ir.novavax.com/node/15506/pdf
38 K, E. et al. Efficacy of ChAdOx1 nCoV-19 (AZD1222) vaccine against SARS-CoV-2 variant of concern 202012/01(B.1.1.7): an exploratory analysis of a randomised controlled trial. Lancet, doi:10.1016/S0140-6736(21)00628-0 (2021).
39 Pfizer (2021) Pfizer and BioNTech confirm high efficacy and no serious safety concerns through up to six months following second dose in updated topline analysis of landmark COVID-19 vaccine study. Press Release Apr. 1, 2021: https://www.pfizer.com/news/press-release/press-release-detail/pfizer-and-biontech-confirm-high-efficacy-and-no-serious.
40 Corbett, K. S. et al. SARS-CoV-2 mRNA vaccine design enabled by prototype pathogen preparedness. Nature 586, 567-571, doi:10.1038/s41586-020-2622-0 (2020).
41 Ho, D. et al. Increased Resistance of SARS-CoV-2 Variants B.1.351 and B.1.1.7 to Antibody Neutralization. Res Sq, doi:10.21203/rs.3.rs-155394/v1 (2021).
42 Wrobel, A. G. et al. SARS-CoV-2 and bat RaTG13 spike glycoprotein structures inform on virus evolution and furin-cleavage effects. Nat Struct Mol Biol 27, 763-767, doi:10.1038/s41594-020-0468-7 (2020).
43 Lan, J. et al. Structure of the SARS-CoV-2 spike receptor-binding domain bound to the ACE2 receptor. Nature 581, 215-220, doi:10.1038/s41586-020-2180-5 (2020).
44 Nelson, J. et al. Impact of mRNA chemistry and manufacturing process on innate immune activation. Sci Adv 6, eaaz6893, doi:10.1126/sciadv.aaz6893 (2020).
45 Hassett, K. J. et al. Optimization of Lipid Nanoparticles for Intramuscular Administration of mRNA Vaccines. Mol Ther Nucleic Acids 15, 1-11, doi:10.1016/j.omtn.2019.01.013 (2019).
46 John, S. et al. Multi-antigenic human cytomegalovirus mRNA vaccines that elicit potent humoral and cell-mediated immunity. Vaccine 36, 1689-1699, doi:10.1016/j.vaccine.2018.01.029 (2018).
47 Bahl, K. et al. Preclinical and Clinical Demonstration of Immunogenicity by mRNA Vaccines against H10N8 and H7N9 Influenza Viruses. Mol Ther 25, 1316-1327, doi:10.1016/j.ymthe.2017.03.035 (2017).
48 Vogel, A. B. et al. Self-Amplifying RNA Vaccines Give Equivalent Protection against Influenza to mRNA Vaccines but at Much Lower Doses. Mol Ther 26, 446-455, doi:10.1016/j.ymthe.2017.11.017 (2018).
49 Whitt, M. A. Generation of VSV pseudotypes using recombinant DeltaG-VSV for studies on virus entry, identification of entry inhibitors, and immune responses to vaccines. J Virol Methods 169, 365-374, doi:10.1016/j.jviromet.2010.08.006 (2010).

### Additional Sequences

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a poly(A) tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 33)
GGGAAAUAAGAGAGAAAAGAAGAGUAAGAAGAAAUAUAAGAGCCACC
5′ UTR:
(SEQ ID NO: 2)
GGGAAAUAAGAGAGAAAAGAAGAGUAAGAAGAAAUAUAAGACCCCGGCG
CCGCCACC
3′ UTR:
(SEQ ID NO: 34)
UGAUAAUAGGCUGGAGCCUCGGUGGCCAUGCUUCUUGCCCCUUGGGCCU
CCCCCCAGCCCCUCCUCCCCUUCCUGCACCCGUACCCCCGUGGUCUUUG
AAUAAAGUCUGAGUGGGCGGC
3′ UTR:
(SEQ ID NO: 4)
UGAUAAUAGGCUGGAGCCUCGGUGGCCUAGCUUCUUGCCCCUUGGGCCU
CCCCCCAGCCCCUCCUCCCCUUCCUGCACCCGUACCCCCGUGGUCUUUG
AAUAAAGUCUGAGUGGGCGGC

TABLE 1
Sequence Listing
NTD-RBD-HATM_E484K

| SEQ ID NO: 18 consists of from 5′ end to 3′ end: 5′ UTR SEQ ID NO: 2, | | 18 |
|---|---|---|
| mRNA ORF SEQ ID NO: 19, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 19 |
| Construct | CAGUGCGUGAACCUGACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCCACGUGAGCGGCACCAACGGCACCAAGCGGUUCG | |
| | ACAACCCCGUGCUGCCCUUCAACGACGGCGUGUACUUCGC | |
| | CAGCACCGAGAAGAGCAACAUCAUCCGGGGCUGGAUCUU | |
| | CGGCACCACCCUGGACAGCAAGACCCAGAGCCUGCUGAUC | |
| | GUGAAUAACGCCACCAACGUGGUGAUCAAGGUGUGCGAG | |
| | UUCCAGUUCUGCAACGACCCCUUCCUGGGCGUGUACUACC | |
| | ACAAGAACAACAAGAGCUGGAUGGAGAGCGAGUUCCGGG | |
| | UGUACAGCAGCGCCAACAACUGCACCUUCGAGUACGUGA | |
| | GCCAGCCCUUCCUGAUGGACCUGGAGGGCAAGCAGGGCA | |
| | ACUUCAAGAACCUGCGGGAGUUCGUGUUCAAGAACAUCG | |
| | ACGGCUACUUCAAGAUCUACAGCAAGCACACCCCAAUCA | |
| | ACCUGGUGCGGGAUCUGCCCCAGGGCUUCUCAGCCCUGG | |
| | AGCCCCUGGUGGACCUGCCCAUCGGCAUCAACAUCACCCG | |
| | GUUCCAGACCCUGCUGGCCCUGCACCGGAGCUACCUGACC | |
| | CCAGGCGACAGCAGCAGCGGGUGGACAGCAGGCGCGGCU | |
| | GCUUACUACGUGGGCUACCUGCAGCCCCGGACCUUCCUGC | |
| | UGAAGUACAACGAGAACGGCACCAUCACCGACGCCGUGG | |
| | ACGGAGGCGGAUCGGGAGGCGGACCCAACAUCACCAACC | |
| | UGUGCCCCUUCGGCGAGGUGUUCAACGCCACCCGGUUCGC | |
| | CAGCGUGUACGCCUGGAACCGGAAGCGGAUCAGCAACUG | |
| | CGUGGCCGACUACAGCGUGCUGUACAACAGCGCCAGCUU | |
| | CAGCACCUUCAAGUGCUACGGCGUGAGCCCCACCAAGCUG | |
| | AACGACCUGUGCUUCACCAACGUGUACGCCGACAGCUUC | |
| | GUGAUCCGUGGCGACGAGGUGCGGCAGAUCGCACCCGGC | |
| | CAGACAGGCAAGAUCGCCGACUACAACUACAAGCUGCCC | |
| | GACGACUUCACCGGCUGCGUGAUCGCCUGGAACAGCAAC | |
| | AACCUCGACAGCAAGGUGGGCGGCAACUACAACUACCUG | |
| | UACCGGCUGUUCCGGAAGAGCAACCUGAAGCCCUUCGAG | |
| | CGGGACAUCAGCACCGAGAUCUACCAAGCCGGCUCCACCC | |
| | CUUGCAACGGCGUGAAGGGCUUCAACUGCUACUUCCCUC | |
| | UGCAGAGCUACGGCUUCCAGCCCACCAACGGCGUGGGCU | |
| | ACCAGCCCUACCGGGUGGUGGUGCUGAGCUUCGAGCUGC | |
| | UGCACGCCCCAGCCACCGUGUGUGGCCCCAAGUCUGGCGG | |
| | AGGCAGCAUCCUGGCCAUCUACAGCACCGUGGCCAGCAGC | |
| | CUGGUGCUGCUGGUGAGCCUGGGCGCCAUCAGCUUC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MFVFLVLLPLVSSQCVNLTTRTQLPPAYTNSFTRGVYYPDKVF | 20 |
| acid sequence | RSSVLHSTQDLFLPFFSNVTWFHAIHVSGTNGTKRFDNPVLPF | |
| | NDGVYFASTEKSNIIRGWIFGTTLDSKTQSLLIVNNATNVVIKV | |
| | CEFQFCNDPFLGVYYHKNNKSWMESEFRVYSSANNCTFEYVS | |
| | QPFLMDLEGKQGNFKNLREFVFKNIDGYFKIYSKHTPINLVRD | |
| | LPQGFSALEPLVDLPIGINITRFQTLLALHRSYLTPGDSSSGWT | |
| | AGAAAYYVGYLQPRTFLLKYNENGTITDAVDGGGSGGGPNIT | |
| | NLCPFGEVENATRFASVYAWNRKRISNCVADYSVLYNSASFS | |
| | TFKCYGVSPTKLNDLCFTNVYADSFVIRGDEVRQIAPGQTGKI | |
| | ADYNYKLPDDFTGCVIAWNSNNLDSKVGGNYNYLYRLFRKS | |
| | NLKPFERDISTEIYQAGSTPCNGVKGFNCYFPLQSYGFQPTNG | |
| | VGYQPYRVVVLSFELLHAPATVCGPKSGGGSILAIYSTVASSL | |
| | VLLVSLGAISF | |
| PolyA tail | 100 nt | |

NTD-RBD-HATM_K417N_E484K_N501Y

| SEQ ID NO: 21 consists of from 5′ end to 3′ end: 5′ UTR SEQ ID NO: 2, | | 21 |
|---|---|---|
| mRNA ORF SEQ ID NO: 22, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 22 |
| Construct | CAGUGCGUGAACCUGACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCCACGUGAGCGGCACCAACGGCACCAAGCGGUUCG | |
| | ACAACCCCGUGCUGCCCUUCAACGACGGCGUGUACUUCGC | |
| | CAGCACCGAGAAGAGCAACAUCAUCCGGGGCUGGAUCUU | |
| | CGGCACCACCCUGGACAGCAAGACCCAGAGCCUGCUGAUC | |
| | GUGAAUAACGCCACCAACGUGGUGAUCAAGGUGUGCGAG | |
| | UUCCAGUUCUGCAACGACCCCUUCCUGGGCGUGUACUACC | |
| | ACAAGAACAACAAGAGCUGGAUGGAGAGCGAGUUCCGGG | |
| | UGUACAGCAGCGCCAACAACUGCACCUUCGAGUACGUGA | |
| | GCCAGCCCUUCCUGAUGGACCUGGAGGGCAAGCAGGGCA | |
| | ACUUCAAGAACCUGCGGGAGUUCGUGUUCAAGAACAUCG | |
| | ACGGCUACUUCAAGAUCUACAGCAAGCACACCCCAAUCA | |
| | ACCUGGUGCGGGAUCUGCCCCAGGGCUUCUCAGCCCUGG | |
| | AGCCCCUGGUGGACCUGCCCAUCGGCAUCAACAUCACCCG | |
| | GUUCCAGACCCUGCUGGCCCUGCACCGGAGCUACCUGACC | |
| | CCAGGCGACAGCAGCAGCGGGUGGACAGCAGGCGCGGCU | |
| | GCUUACUACGUGGGCUACCUGCAGCCCCGGACCUUCCUGC | |
| | UGAAGUACAACGAGAACGGCACCAUCACCGACGCCGUGG | |
| | ACGGAGGCGGAUCGGGAGGCGGACCCAACAUCACCAACC | |
| | UGUGCCCCUUCGGCGAGGUGUUCAACGCCACCCGGUUCGC | |
| | CAGCGUGUACGCCUGGAACCGGAAGCGGAUCAGCAACUG | |
| | CGUGGCCGACUACAGCGUGCUGUACAACAGCGCCAGCUU | |
| | CAGCACCUUCAAGUGCUACGGCGUGAGCCCCACCAAGCUG | |
| | AACGACCUGUGCUUCACCAACGUGUACGCCGACAGCUUC | |
| | GUGAUCCGUGGCGACGAGGUGCGGCAGAUCGCACCCGGC | |
| | CAGACAGGCAACAUCGCCGACUACAACUACAAGCUGCCCG | |
| | ACGACUUCACCGGCUGCGUGAUCGCCUGGAACAGCAACA | |
| | ACCUCGACAGCAAGGUGGGCGGCAACUACAACUACCUGU | |
| | ACCGGCUGUUCCGGAAGAGCAACCUGAAGCCCUUCGAGC | |
| | GGGACAUCAGCACCGAGAUCUACCAAGCCGGCUCCACCCC | |
| | UUGCAACGGCGUGAAGGGCUUCAACUGCUACUUCCCUCU | |
| | GCAGAGCUACGGCUUCCAGCCCACCUACGGCGUGGGCUAC | |
| | CAGCCCUACCGGGUGGUGGUGCUGAGCUUCGAGCUGCUG | |
| | CACGCCCCAGCCACCGUGUGUGGCCCCAAGUCUGGCGGAG | |
| | GCAGCAUCCUGGCCAUCUACAGCACCGUGGCCAGCAGCCU | |
| | GGUGCUGCUGGUGAGCCUGGGCGCCAUCAGCUUC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MFVFLVLLPLVSSQCVNLTTRTQLPPAYTNSFTRGVYYPDKVF | 23 |
| acid sequence | RSSVLHSTQDLFLPFFSNVTWFHAIHVSGTNGTKRFDNPVLPF | |
| | NDGVYFASTEKSNIIRGWIFGTTLDSKTQSLLIVNNATNVVIKV | |
| | CEFQFCNDPFLGVYYHKNNKSWMESEFRVYSSANNCTFEYVS | |
| | QPFLMDLEGKQGNFKNLREFVFKNIDGYFKIYSKHTPINLVRD | |
| | LPQGFSALEPLVDLPIGINITRFQTLLALHRSYLTPGDSSSGWT | |
| | AGAAAYYVGYLQPRTFLLKYNENGTITDAVDGGGSGGGPNIT | |
| | NLCPFGEVENATRFASVYAWNRKRISNCVADYSVLYNSASFS | |
| | TFKCYGVSPTKLNDLCFTNVYADSFVIRGDEVRQIAPGQTGNI | |
| | ADYNYKLPDDFTGCVIAWNSNNLDSKVGGNYNYLYRLFRKS | |
| | NLKPFERDISTEIYQAGSTPCNGVKGFNCYFPLQSYGFQPTYG | |
| | VGYQPYRVVVLSFELLHAPATVCGPKSGGGSILAIYSTVASSL | |
| | VLLVSLGAISF | |
| PolyA tail | 100 nt | |

NTD-RBD-HATM_(RSA) L18F_D80A_D215G_L242_244del_R246I_K417N_E484K_N501Y

| SEQ ID NO: 24 consists of from 5′ end to 3′ end: 5′ UTR SEQ ID NO: 2, | | 24 |
|---|---|---|
| mRNA ORF SEQ ID NO: 25, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 25 |
| Construct | CAGUGCGUGAACUUUACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCCACGUGAGCGGCACCAACGGCACCAAGCGGUUCG | |
| | CCAACCCCGUGCUGCCCUUCAACGACGGCGUGUACUUCGC | |
| | CAGCACCGAGAAGAGCAACAUCAUCCGGGGCUGGAUCUU | |
| | CGGCACCACCCUGGACAGCAAGACCCAGAGCCUGCUGAUC | |
| | GUGAAUAACGCCACCAACGUGGUGAUCAAGGUGUGCGAG | |
| | UUCCAGUUCUGCAACGACCCCUUCCUGGGCGUGUACUACC | |
| | ACAAGAACAACAAGAGCUGGAUGGAGAGCGAGUUCCGGG | |
| | UGUACAGCAGCGCCAACAACUGCACCUUCGAGUACGUGA | |
| | GCCAGCCCUUCCUGAUGGACCUGGAGGGCAAGCAGGGCA | |
| | ACUUCAAGAACCUGCGGGAGUUCGUGUUCAAGAACAUCG | |
| | ACGGCUACUUCAAGAUCUACAGCAAGCACACCCCAAUCA | |
| | ACCUGGUGCGGGGCCUGCCCCAGGGCUUCUCAGCCCUGGA | |
| | GCCCCUGGUGGACCUGCCCAUCGGCAUCAACAUCACCCGG | |
| | UUCCAGACCCUGCACAUCAGCUACCUGACCCCAGGCGACA | |
| | GCAGCAGCGGGUGGACAGCAGGCGCGGCUGCUUACUACG | |
| | UGGGCUACCUGCAGCCCCGGACCUUCCUGCUGAAGUACA | |
| | ACGAGAACGGCACCAUCACCGACGCCGUGGACGGAGGCG | |
| | GAUCGGGAGGCGGACCCAACAUCACCAACCUGUGCCCCUU | |
| | CGGCGAGGUGUUCAACGCCACCCGGUUCGCCAGCGUGUA | |
| | CGCCUGGAACCGGAAGCGGAUCAGCAACUGCGUGGCCGA | |
| | CUACAGCGUGCUGUACAACAGCGCCAGCUUCAGCACCUUC | |
| | AAGUGCUACGGCGUGAGCCCCACCAAGCUGAACGACCUG | |
| | UGCUUCACCAACGUGUACGCCGACAGCUUCGUGAUCCGU | |
| | GGCGACGAGGUGCGGCAGAUCGCACCCGGCCAGACAGGC | |
| | AACAUCGCCGACUACAACUACAAGCUGCCCGACGACUUCA | |
| | CCGGCUGCGUGAUCGCCUGGAACAGCAACAACCUCGACA | |
| | GCAAGGUGGGCGGCAACUACAACUACCUGUACCGGCUGU | |
| | UCCGGAAGAGCAACCUGAAGCCCUUCGAGCGGGACAUCA | |
| | GCACCGAGAUCUACCAAGCCGGCUCCACCCCUUGCAACGG | |
| | CGUGAAGGGCUUCAACUGCUACUUCCCUCUGCAGAGCUA | |
| | CGGCUUCCAGCCCACCUACGGCGUGGGCUACCAGCCCUAC | |
| | CGGGUGGUGGUGCUGAGCUUCGAGCUGCUGCACGCCCCA | |
| | GCCACCGUGUGUGGCCCCAAGUCUGGCGGAGGCAGCAUC | |
| | CUGGCCAUCUACAGCACCGUGGCCAGCAGCCUGGUGCUGC | |
| | UGGUGAGCCUGGGCGCCAUCAGCUUC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MFVFLVLLPLVSSQCVNFTTRTQLPPAYTNSFTRGVYYPDKVF | 26 |
| acid sequence | RSSVLHSTQDLFLPFFSNVTWFHAIHVSGTNGTKRFANPVLPF | |
| | NDGVYFASTEKSNIIRGWIFGTTLDSKTQSLLIVNNATNVVIKV | |
| | CEFQFCNDPFLGVYYHKNNKSWMESEFRVYSSANNCTFEYVS | |
| | QPFLMDLEGKQGNFKNLREFVFKNIDGYFKIYSKHTPINLVRG | |
| | LPQGFSALEPLVDLPIGINITRFQTLHISYLTPGDSSSGWTAGAA | |
| | AYYVGYLQPRTFLLKYNENGTITDAVDGGGSGGGPNITNLCP | |
| | FGEVFNATRFASVYAWNRKRISNCVADYSVLYNSASFSTFKC | |
| | YGVSPTKLNDLCFTNVYADSFVIRGDEVRQIAPGQTGNIADYN | |
| | YKLPDDFTGCVIAWNSNNLDSKVGGNYNYLYRLFRKSNLKPF | |
| | ERDISTEIYQAGSTPCNGVKGFNCYFPLQSYGFQPTYGVGYQP | |
| | YRVVVLSFELLHAPATVCGPKSGGGSILAIYSTVASSLVLLVSL | |
| | GAISF | |
| PolyA tail | 100 nt | |
| NTD-RBD-HATM_(UK) H69del_V70del_Y144del_N501Y | | |
| SEQ ID NO: 27 consists of from 5′ end to 3′ end: 5′ UTR SEQ ID NO: 2, | | 27 |
| mRNA ORF SEQ ID NO: 28, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 28 |
| Construct | CAGUGCGUGAACCUGACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCAGCGGCACCAACGGCACCAAGCGGUUCGACAACC | |
| | CCGUGCUGCCCUUCAACGACGGCGUGUACUUCGCCAGCAC | |
| | CGAGAAGAGCAACAUCAUCCGGGGCUGGAUCUUCGGCAC | |
| | CACCCUGGACAGCAAGACCCAGAGCCUGCUGAUCGUGAA | |
| | UAACGCCACCAACGUGGUGAUCAAGGUGUGCGAGUUCCA | |
| | GUUCUGCAACGACCCCUUCCUGGGCGUGUACCACAAGAA | |
| | CAACAAGAGCUGGAUGGAGAGCGAGUUCCGGGUGUACAG | |
| | CAGCGCCAACAACUGCACCUUCGAGUACGUGAGCCAGCCC | |
| | UUCCUGAUGGACCUGGAGGGCAAGCAGGGCAACUUCAAG | |
| | AACCUGCGGGAGUUCGUGUUCAAGAACAUCGACGGCUAC | |
| | UUCAAGAUCUACAGCAAGCACACCCCAAUCAACCUGGUG | |
| | CGGGAUCUGCCCCAGGGCUUCUCAGCCCUGGAGCCCCUGG | |
| | UGGACCUGCCCAUCGGCAUCAACAUCACCCGGUUCCAGAC | |
| | CCUGCUGGCCCUGCACCGGAGCUACCUGACCCCAGGCGAC | |
| | AGCAGCAGCGGGUGGACAGCAGGCGCGGCUGCUUACUAC | |
| | GUGGGCUACCUGCAGCCCCGGACCUUCCUGCUGAAGUAC | |
| | AACGAGAACGGCACCAUCACCGACGCCGUGGACGGAGGC | |
| | GGAUCGGGAGGCGGACCCAACAUCACCAACCUGUGCCCCU | |
| | UCGGCGAGGUGUUCAACGCCACCCGGUUCGCCAGCGUGU | |
| | ACGCCUGGAACCGGAAGCGGAUCAGCAACUGCGUGGCCG | |
| | ACUACAGCGUGCUGUACAACAGCGCCAGCUUCAGCACCU | |
| | UCAAGUGCUACGGCGUGAGCCCCACCAAGCUGAACGACC | |
| | UGUGCUUCACCAACGUGUACGCCGACAGCUUCGUGAUCC | |
| | GUGGCGACGAGGUGCGGCAGAUCGCACCCGGCCAGACAG | |
| | GCAAGAUCGCCGACUACAACUACAAGCUGCCCGACGACU | |
| | UCACCGGCUGCGUGAUCGCCUGGAACAGCAACAACCUCG | |
| | ACAGCAAGGUGGGCGGCAACUACAACUACCUGUACCGGC | |
| | UGUUCCGGAAGAGCAACCUGAAGCCCUUCGAGCGGGACA | |
| | UCAGCACCGAGAUCUACCAAGCCGGCUCCACCCCUUGCAA | |
| | CGGCGUGGAGGGCUUCAACUGCUACUUCCCUCUGCAGAG | |
| | CUACGGCUUCCAGCCCACCUACGGCGUGGGCUACCAGCCC | |
| | UACCGGGUGGUGGUGCUGAGCUUCGAGCUGCUGCACGCC | |
| | CCAGCCACCGUGUGUGGCCCCAAGUCUGGCGGAGGCAGC | |
| | AUCCUGGCCAUCUACAGCACCGUGGCCAGCAGCCUGGUGC | |
| | UGCUGGUGAGCCUGGGCGCCAUCAGCUUC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MFVFLVLLPLVSSQCVNLTTRTQLPPAYTNSFTRGVYYPDKVF | 29 |
| acid sequence | RSSVLHSTQDLFLPFFSNVTWFHAISGTNGTKRFDNPVLPEND | |
| | GVYFASTEKSNIIRGWIFGTTLDSKTQSLLIVNNATNVVIKVCE | |
| | FQFCNDPFLGVYHKNNKSWMESEFRVYSSANNCTFEYVSQPF | |
| | LMDLEGKQGNFKNLREFVFKNIDGYFKIYSKHTPINLVRDLPQ | |
| | GFSALEPLVDLPIGINITRFQTLLALHRSYLTPGDSSSGWTAGA | |
| | AAYYVGYLQPRTFLLKYNENGTITDAVDGGGSGGGPNITNLC | |
| | PFGEVFNATRFASVYAWNRKRISNCVADYSVLYNSASFSTFK | |
| | CYGVSPTKLNDLCFTNVYADSFVIRGDEVRQIAPGQTGKIADY | |
| | NYKLPDDFTGCVIAWNSNNLDSKVGGNYNYLYRLFRKSNLK | |
| | PFERDISTEIYQAGSTPCNGVEGENCYFPLQSYGFQPTYGVGY | |
| | QPYRVVVLSFELLHAPATVCGPKSGGGSILAIYSTVASSLVLL | |
| | VSLGAISF | |
| PolyA tail | 100 nt | |

NTD-RBD-HATM_(RSA+UK)
L18F_H69del_V70del_D80A_Y144del_D215G_L242_244del_R246I_K417N_E484K_N501Y

| SEQ ID NO: 30 consists of from 5′ end to 3′ end: 5′ UTR SEQ ID NO: 2, | | 30 |
|---|---|---|
| mRNA ORF SEQ ID NO: 31, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGACCCCGGCGCCGCCACC | |
| ORF of mRNA | AUGUUCGUGUUCCUGGUGCUGCUGCCCCUGGUGAGCAGC | 31 |
| Construct | CAGUGCGUGAACUUUACCACCCGGACCCAGCUGCCACCAG | |
| (excluding the stop | CCUACACCAACAGCUUCACCCGGGGCGUCUACUACCCCGA | |
| codon) | CAAGGUGUUCCGGAGCAGCGUCCUGCACAGCACCCAGGA | |
| | CCUGUUCCUGCCCUUCUUCAGCAACGUGACCUGGUUCCAC | |
| | GCCAUCAGCGGCACCAACGGCACCAAGCGGUUCGCCAACC | |
| | CCGUGCUGCCCUUCAACGACGGCGUGUACUUCGCCAGCAC | |
| | CGAGAAGAGCAACAUCAUCCGGGGCUGGAUCUUCGGCAC | |
| | CACCCUGGACAGCAAGACCCAGAGCCUGCUGAUCGUGAA | |
| | UAACGCCACCAACGUGGUGAUCAAGGUGUGCGAGUUCCA | |
| | GUUCUGCAACGACCCCUUCCUGGGCGUGUACCACAAGAA | |
| | CAACAAGAGCUGGAUGGAGAGCGAGUUCCGGGUGUACAG | |
| | CAGCGCCAACAACUGCACCUUCGAGUACGUGAGCCAGCCC | |
| | UUCCUGAUGGACCUGGAGGGCAAGCAGGGCAACUUCAAG | |
| | AACCUGCGGGAGUUCGUGUUCAAGAACAUCGACGGCUAC | |
| | UUCAAGAUCUACAGCAAGCACACCCCAAUCAACCUGGUG | |
| | CGGGGCCUGCCCCAGGGCUUCUCAGCCCUGGAGCCCCUGG | |
| | UGGACCUGCCCAUCGGCAUCAACAUCACCCGGUUCCAGAC | |
| | CCUGCACAUCAGCUACCUGACCCCAGGCGACAGCAGCAGC | |
| | GGGUGGACAGCAGGCGCGGCUGCUUACUACGUGGGCUAC | |
| | CUGCAGCCCCGGACCUUCCUGCUGAAGUACAACGAGAAC | |
| | GGCACCAUCACCGACGCCGUGGACGGAGGCGGAUCGGGA | |
| | GGCGGACCCAACAUCACCAACCUGUGCCCCUUCGGCGAGG | |
| | UGUUCAACGCCACCCGGUUCGCCAGCGUGUACGCCUGGA | |
| | ACCGGAAGCGGAUCAGCAACUGCGUGGCCGACUACAGCG | |
| | UGCUGUACAACAGCGCCAGCUUCAGCACCUUCAAGUGCU | |
| | ACGGCGUGAGCCCCACCAAGCUGAACGACCUGUGCUUCAC | |
| | CAACGUGUACGCCGACAGCUUCGUGAUCCGUGGCGACGA | |
| | GGUGCGGCAGAUCGCACCCGGCCAGACAGGCAACAUCGCC | |
| | GACUACAACUACAAGCUGCCCGACGACUUCACCGGCUGCG | |
| | UGAUCGCCUGGAACAGCAACAACCUCGACAGCAAGGUGG | |
| | GCGGCAACUACAACUACCUGUACCGGCUGUUCCGGAAGA | |
| | GCAACCUGAAGCCCUUCGAGCGGGACAUCAGCACCGAGA | |
| | UCUACCAAGCCGGCUCCACCCCUUGCAACGGCGUGAAGGG | |
| | CUUCAACUGCUACUUCCCUCUGCAGAGCUACGGCUUCCAG | |
| | CCCACCUACGGCGUGGGCUACCAGCCCUACCGGGUGGUGG | |
| | UGCUGAGCUUCGAGCUGCUGCACGCCCCAGCCACCGUGUG | |
| | UGGCCCCAAGUCUGGCGGAGGCAGCAUCCUGGCCAUCUA | |
| | CAGCACCGUGGCCAGCAGCCUGGUGCUGCUGGUGAGCCU | |
| | GGGCGCCAUCAGCUUC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MFVFLVLLPLVSSQCVNFTTRTQLPPAYTNSFTRGVYYPDKVF | 32 |
| acid sequence | RSSVLHSTQDLFLPFFSNVTWFHAISGTNGTKRFANPVLPEND | |
| | GVYFASTEKSNIIRGWIFGTTLDSKTQSLLIVNNATNVVIKVCE | |
| | FQFCNDPFLGVYHKNNKSWMESEFRVYSSANNCTFEYVSQPF | |
| | LMDLEGKQGNFKNLREFVFKNIDGYFKIYSKHTPINLVRGLPQ | |
| | GFSALEPLVDLPIGINITRFQTLHISYLTPGDSSSGWTAGAAAY | |
| | YVGYLQPRTFLLKYNENGTITDAVDGGGSGGGPNITNLCPFGE | |
| | VFNATRFASVYAWNRKRISNCVADYSVLYNSASFSTFKCYGV | |
| | SPTKLNDLCFTNVYADSFVIRGDEVRQIAPGQTGNIADYNYKL | |
| | PDDFTGCVIAWNSNNLDSKVGGNYNYLYRLFRKSNLKPFERD | |
| | ISTEIYQAGSTPCNGVKGFNCYFPLQSYGFQPTYGVGYQPYRV | |
| | VVLSFELLHAPATVCGPKSGGGSILAIYSTVASSLVLLVSLGAI | |
| | SF | |
| PolyA tail | 100 nt | |

SARS-CoV-2 Wild Type Spike (S) Protein

| SEQ ID NO: 39 consists of from 5′ end to 3′ end: 5′ UTR SEQ ID NO: 2, | | 39 |
|---|---|---|
| mRNA ORF SEQ ID NO: 40, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 40 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCACCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGGCGUGUACUACCACAAGAACAACAAGAGCUGGAUGG | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGUGGAGAAGGGCAUCUACCAGACCAGCAACUUCCGGGUGCAGCCC | |
| | ACCGAGAGCAUCGUGCGGUUCCCCAACAUCACCAACCUGUGCCCCU | |
| | UCGGCGAGGUGUUCAACGCCACCCGGUUCGCCAGCGUGUACGCCUG | |
| | GAACCGGAAGCGGAUCAGCAACUGCGUGGCCGACUACAGCGUGCUG | |
| | UACAACAGCGCCAGCUUCAGCACCUUCAAGUGCUACGGCGUGAGCC | |
| | CCACCAAGCUGAACGACCUGUGCUUCACCAACGUGUACGCCGACAG | |
| | CUUCGUGAUCCGUGGCGACGAGGUGCGGCAGAUCGCACCCGGCCAG | |
| | ACAGGCAAGAUCGCCGACUACAACUACAAGCUGCCCGACGACUUCA | |
| | CCGGCUGCGUGAUCGCCUGGAACAGCAACAACCUCGACAGCAAGGU | |
| | GGGCGGCAACUACAACUACCUGUACCGGCUGUUCCGGAAGAGCAAC | |
| | CUGAAGCCCUUCGAGCGGGACAUCAGCACCGAGAUCUACCAAGCCG | |
| | GCUCCACCCCUUGCAACGGCGUGGAGGGCUUCAACUGCUACUUCCC | |
| | UCUGCAGAGCUACGGCUUCCAGCCCACCAACGGCGUGGGCUACCAG | |
| | CCCUACCGGGUGGUGGUGCUGAGCUUCGAGCUGCUGCACGCCCCAG | |
| | CCACCGUGUGUGGCCCCAAGAAGAGCACCAACCUGGUGAAGAACAA | |
| | GUGCGUGAACUUCAACUUCAACGGCCUUACCGGCACCGGCGUGCUG | |
| | ACCGAGAGCAACAAGAAAUUCCUGCCCUUUCAGCAGUUCGGCCGGG | |
| | ACAUCGCCGACACCACCGACGCUGUGCGGGAUCCCCAGACCCUGGA | |
| | GAUCCUGGACAUCACCCCUUGCAGCUUCGGCGGCGUGAGCGUGAUC | |
| | ACCCCAGGCACCAACACCAGCAACCAGGUGGCCGUGCUGUACCAGG | |
| | ACGUGAACUGCACCGAGGUGCCCGUGGCCAUCCACGCCGACCAGCU | |
| | GACACCCACCUGGCGGGUCUACAGCACCGGCAGCAACGUGUUCCAG | |
| | ACCCGGGCCGGUUGCCUGAUCGGCGCCGAGCACGUGAACAACAGCU | |
| | ACGAGUGCGACAUCCCCAUCGGCGCCGGCAUCUGUGCCAGCUACCA | |
| | GACCCAGACCAAUUCACCCCGGAGGGCAAGGAGCGUGGCCAGCCAG | |
| | AGCAUCAUCGCCUACACCAUGAGCCUGGGCGCCGAGAACAGCGUGG | |
| | CCUACAGCAACAACAGCAUCGCCAUCCCCACCAACUUCACCAUCAG | |
| | CGUGACCACCGAGAUUCUGCCCGUGAGCAUGACCAAGACCAGCGUG | |
| | GACUGCACCAUGUACAUCUGCGGCGACAGCACCGAGUGCAGCAACC | |
| | UGCUGCUGCAGUACGGCAGCUUCUGCACCCAGCUGAACCGGGCCCU | |
| | GACCGGCAUCGCCGUGGAGCAGGACAAGAACACCCAGGAGGUGUUC | |
| | GCCCAGGUGAAGCAGAUCUACAAGACCCCUCCCAUCAAGGACUUCG | |
| | GCGGCUUCAACUUCAGCCAGAUCCUGCCCGACCCCAGCAAGCCCAG | |
| | CAAGCGGAGCUUCAUCGAGGACCUGCUGUUCAACAAGGUGACCCUA | |
| | GCCGACGCCGGCUUCAUCAAGCAGUACGGCGACUGCCUCGGCGACA | |
| | UAGCCGCCCGGGACCUGAUCUGCGCCCAGAAGUUCAACGGCCUGAC | |
| | CGUGCUGCCUCCCCUGCUGACCGACGAGAUGAUCGCCCAGUACACC | |
| | AGCGCCCUGUUAGCCGGAACCAUCACCAGCGGCUGGACUUUCGGCG | |
| | CUGGAGCCGCUCUGCAGAUCCCCUUCGCCAUGCAGAUGGCCUACCG | |
| | GUUCAACGGCAUCGGCGUGACCCAGAACGUGCUGUACGAGAACCAG | |
| | AAGCUGAUCGCCAACCAGUUCAACAGCGCCAUCGGCAAGAUCCAGG | |
| | ACAGCCUGAGCAGCACCGCUAGCGCCCUGGGCAAGCUGCAGGACGU | |
| | GGUGAACCAGAACGCCCAGGCCCUGAACACCCUGGUGAAGCAGCUG | |
| | AGCAGCAACUUCGGCGCCAUCAGCAGCGUGCUGAACGACAUCCUGA | |
| | GCCGGCUGGACAAGGUGGAGGCCGAGGUGCAGAUCGACCGGCUGAU | |
| | CACUGGCCGGCUGCAGAGCCUGCAGACCUACGUGACCCAGCAGCUG | |
| | AUCCGGGCCGCCGAGAUUCGGGCCAGCGCCAACCUGGCCGCCACCA | |
| | AGAUGAGCGAGUGCGUGCUGGGCCAGAGCAAGCGGGUGGACUUCUG | |
| | CGGCAAGGGCUACCACCUGAUGAGCUUUCCCCAGAGCGCACCCCAC | |
| | GGAGUGGUGUUCCUGCACGUGACCUACGUGCCCGCCCAGGAGAAGA | |
| | ACUUCACCACCGCCCCAGCCAUCUGCCACGACGGCAAGGCCCACUU | |
| | UCCCCGGGAGGGCGUGUUCGUGAGCAACGGCACCCACUGGUUCGUG | |
| | ACCCAGCGGAACUUCUACGAGCCCCAGAUCAUCACCACCGACAACA | |
| | CCUUCGUGAGCGGCAACUGCGACGUGGUGAUCGGCAUCGUGAACAA | |
| | CACCGUGUACGAUCCCCUGCAGCCCGAGCUGGACAGCUUCAAGGAG | |
| | GAGCUGGACAAGUACUUCAAGAAUCACACCAGCCCCGACGUGGACC | |
| | UGGGCGACAUCAGCGGCAUCAACGCCAGCGUGGUGAACAUCCAGAA | |
| | GGAGAUCGAUCGGCUGAACGAGGUGGCCAAGAACCUGAACGAGAGC | |
| | CUGAUCGACCUGCAGGAGCUGGGCAAGUACGAGCAGUACAUCAAGU | |
| | GGCCCUGGUACAUCUGGCUGGGCUUCAUCGCCGGCCUGAUCGCCAU | |
| | CGUGAUGGUGACCAUCAUGCUGUGCUGCAUGACCAGCUGCUGCAGC | |
| | UGCCUGAAGGGCUGUUGCAGCUGCGGCAGCUGCUGCAAGUUCGACG | |
| | AGGACGACAGCGAGCCCGUGCUGAAGGGCGUGAAGCUGCACUACAC | |
| | C | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVFRSS | 36 |
| acid sequence | VLHSTQDLFLPFFSNVTWFHAIHVSGINGTKRFDNPVLPFNDGVYF | |
| | ASTEKSNIIRGWIFGTTLDSKTQSLLIVNNATNVVIKVCEFQFCND | |
| | PFLGVYYHKNNKSWMESEFRVYSSANNCTFEYVSQPFLMDLEGKOG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTVEKGIYQTSNFRVQP | |
| | TESIVRFPNITNLCPFGEVENATRFASVYAWNRKRISNCVADYSVL | |
| | YNSASFSTFKCYGVSPTKLNDLCFTNVYADSFVIRGDEVRQIAPGQ | |
| | TGKIADYNYKLPDDFTGCVIAWNSNNLDSKVGGNYNYLYRLFRKSN | |
| | LKPFERDISTEIYQAGSTPCNGVEGENCYFPLQSYGFQPTNGVGYQ | |
| | PYRVVVLSFELLHAPATVCGPKKSTNLVKNKCVNFNFNGLTGTGVL | |
| | TESNKKFLPFQQFGRDIADTTDAVRDPQTLEILDITPCSFGGVSVI | |
| | TPGTNTSNQVAVLYQDVNCTEVPVAIHADQLTPTWRVYSTGSNVFQ | |
| | TRAGCLIGAEHVNNSYECDIPIGAGICASYQTQTNSPRRARSVASQ | |
| | SIIAYTMSLGAENSVAYSNNSIAIPTNFTISVTTEILPVSMTKTSV | |
| | DCTMYICGDSTECSNLLLQYGSFCTQLNRALTGIAVEQDKNTQEVE | |
| | AQVKQIYKTPPIKDFGGFNFSQILPDPSKPSKRSFIEDLLFNKVTL | |
| | ADAGFIKQYGDCLGDIAARDLICAQKFNGLTVLPPLLTDEMIAQYT | |
| | SALLAGTITSGWTFGAGAALQIPFAMQMAYRENGIGVTQNVLYENQ | |
| | KLIANQFNSAIGKIQDSLSSTASALGKLQDVVNQNAQALNTLVKQL | |
| | SSNFGAISSVLNDILSRLDKVEAEVQIDRLITGRLQSLQTYVTQQL | |
| | IRAAEIRASANLAATKMSECVLGQSKRVDFCGKGYHLMSFPQSAPH | |
| | GVVFLHVTYVPAQEKNFTTAPAICHDGKAHFPREGVFVSNGTHWFV | |
| | TORNFYEPQIITTDNTFVSGNCDVVIGIVNNTVYDPLQPELDSFKE | |
| | ELDKYFKNHTSPDVDLGDISGINASVVNIQKEIDRLNEVAKNLNES | |
| | LIDLQELGKYEQYIKWPWYIWLGFIAGLIAIVMVTIMLCCMTSCCS | |
| | CLKGCCSCGSCCKFDEDDSEPVLKGVKLHYT | |
| PolyA tail | 100 nt | |

NTD_RBD_TM_IN_[T95I, G142D, E154K, L452R, E484Q]

| SEQ ID NO: 41 consists of from 5′ end to 3′ end: 5′ UTR SEQ ID NO: 2, | | 41 |
|---|---|---|
| mRNA ORF SEQ ID NO: 42, and 3′ UTR SEQ ID NO: 4. | | |
| Chemistry | 1 -methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 42 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCAUCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGACGUGUACUACCACAAGAACAACAAGAGCUGGAUGA | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACGGAG | |
| | GCGGAUCGGGAGGCGGACCCAACAUCACCAACCUGUGCCCCUUCGG | |
| | CGAGGUGUUCAACGCCACCCGGUUCGCCAGCGUGUACGCCUGGAAC | |
| | CGGAAGCGGAUCAGCAACUGCGUGGCCGACUACAGCGUGCUGUACA | |
| | ACAGCGCCAGCUUCAGCACCUUCAAGUGCUACGGCGUGAGCCCCAC | |
| | CAAGCUGAACGACCUGUGCUUCACCAACGUGUACGCCGACAGCUUC | |
| | GUGAUCCGUGGCGACGAGGUGCGGCAGAUCGCACCCGGCCAGACAG | |
| | GCAAGAUCGCCGACUACAACUACAAGCUGCCCGACGACUUCACCGG | |
| | CUGCGUGAUCGCCUGGAACAGCAACAACCUCGACAGCAAGGUGGGC | |
| | GGCAACUACAACUACAGAUACCGGCUGUUCCGGAAGAGCAACCUGA | |
| | AGCCCUUCGAGCGGGACAUCAGCACCGAGAUCUACCAAGCCGGCUC | |
| | CACCCCUUGCAACGGCGUGCAGGGCUUCAACUGCUACUUCCCUCUG | |
| | CAGAGCUACGGCUUCCAGCCCACCAACGGCGUGGGCUACCAGCCCU | |
| | ACCGGGUGGUGGUGCUGAGCUUCGAGCUGCUGCACGCCCCAGCCAC | |
| | CGUGUGUGGCCCCAAGUCUGGCGGAGGCAGCAUCCUGGCCAUCUAC | |
| | AGCACCGUGGCCAGCAGCCUGGUGCUGCUGGUGAGCCUGGGCGCCA | |
| | UCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVFRSS | 43 |
| acid sequence | VLHSTQDLFLPFFSNVTWFHAIHVSGTNGTKRFDNPVLPFNDGVYF | |
| | ASIEKSNIIRGWIFGTTLDSKTQSLLIVNNATNVVIKVCEFQFCND | |
| | PFLDVYYHKNNKSWMKSEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDgggsgggPNITNLCPFGEVENATRFASVYAWN | |
| | RKRISNCVADYSVLYNSASFSTFKCYGVSPTKLNDLCFTNVYADSF | |
| | VIRGDEVRQIAPGQTGKIADYNYKLPDDFTGCVIAWNSNNLDSKVG | |
| | GNYNYRYRLFRKSNLKPFERDISTEIYQAGSTPCNGVQGENCYFPL | |
| | QSYGFQPTNGVGYQPYRVVVLSFELLHAPATVCGPKsgggsilaiy | |
| | stvasslvllvslgaisf | |
| PolyA tail | 100 nt | |

NTD_ext-RBD_ext-TM_IN_[T95I, G142D, E154K, L452R, E484Q]

| SEQ ID NO: 44 consists of from 5′ end to 3′ end: 5′ UTR SEQ ID NO: 2, | | 44 |
|---|---|---|
| mRNA ORF SEQ ID NO: 45, and 3′ UTR SEQ ID NO: 4. | | |
| Chemistry | 1 -methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 45 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGCCAUCCACGUGAGCGGCACCAACGGCACCAA | |
| | GCGGUUCGACAACCCCGUGCUGCCCUUCAACGACGGCGUGUACUUC | |
| | GCCAGCAUCGAGAAGAGCAACAUCAUCCGGGGCUGGAUCUUCGGCA | |
| | CCACCCUGGACAGCAAGACCCAGAGCCUGCUGAUCGUGAAUAACGC | |
| | CACCAACGUGGUGAUCAAGGUGUGCGAGUUCCAGUUCUGCAACGAC | |
| | CCCUUCCUGGACGUGUACUACCACAAGAACAACAAGAGCUGGAUGA | |
| | AGAGCGAGUUCCGGGUGUACAGCAGCGCCAACAACUGCACCUUCGA | |
| | GUACGUGAGCCAGCCCUUCCUGAUGGACCUGGAGGGCAAGCAGGGC | |
| | AACUUCAAGAACCUGCGGGAGUUCGUGUUCAAGAACAUCGACGGCU | |
| | ACUUCAAGAUCUACAGCAAGCACACCCCAAUCAACCUGGUGCGGGA | |
| | UCUGCCCCAGGGCUUCUCAGCCCUGGAGCCCCUGGUGGACCUGCCC | |
| | AUCGGCAUCAACAUCACCCGGUUCCAGACCCUGCUGGCCCUGCACC | |
| | GGAGCUACCUGACCCCAGGCGACAGCAGCAGCGGGUGGACAGCAGG | |
| | CGCGGCUGCUUACUACGUGGGCUACCUGCAGCCCCGGACCUUCCUG | |
| | CUGAAGUACAACGAGAACGGCACCAUCACCGACGCCGUGGACUGCG | |
| | CCCUGGACCCUCUGAGCGAGACCAAGUGCACCCUGAAGAGCUUCAC | |
| | CGGAGGCGGAUCGGGAGGCGGACAGCGGGUGCAGCCCACCGAGAGC | |
| | AUCGUGCGGUUCCCCAACAUCACCAACCUGUGCCCCUUCGGCGAGG | |
| | UGUUCAACGCCACCCGGUUCGCCAGCGUGUACGCCUGGAACCGGAA | |
| | GCGGAUCAGCAACUGCGUGGCCGACUACAGCGUGCUGUACAACAGC | |
| | GCCAGCUUCAGCACCUUCAAGUGCUACGGCGUGAGCCCCACCAAGC | |
| | UGAACGACCUGUGCUUCACCAACGUGUACGCCGACAGCUUCGUGAU | |
| | CCGUGGCGACGAGGUGCGGCAGAUCGCACCCGGCCAGACAGGCAAG | |
| | AUCGCCGACUACAACUACAAGCUGCCCGACGACUUCACCGGCUGCG | |
| | UGAUCGCCUGGAACAGCAACAACCUCGACAGCAAGGUGGGCGGCAA | |
| | CUACAACUACAGAUACCGGCUGUUCCGGAAGAGCAACCUGAAGCCC | |
| | UUCGAGCGGGACAUCAGCACCGAGAUCUACCAAGCCGGCUCCACCC | |
| | CUUGCAACGGCGUGCAGGGCUUCAACUGCUACUUCCCUCUGCAGAG | |
| | CUACGGCUUCCAGCCCACCAACGGCGUGGGCUACCAGCCCUACCGG | |
| | GUGGUGGUGCUGAGCUUCGAGCUGCUGCACGCCCCAGCCACCGUGU | |
| | GUGGCCCCAAGAAGAGCACCAACCUGGUGAAGAACAAGUGCGUGAA | |
| | CUUCAACUUCAACGGCCUUACCGGCACCGGCGUGCUGACCGAGAGC | |
| | AACAAGAAAUUCCUGCCCUUUCAGCAGUUCGGCCGGGACAUCGCCG | |
| | ACACCACCGACGCUGUGCGGGAUCCCCAGACCCUGGAGAUCCUGGA | |
| | CAUCACCCCUUGCAGCUCUGGCGGAGGCAGCAUCCUGGCCAUCUAC | |
| | AGCACCGUGGCCAGCAGCCUGGUGCUCCUGGUGAGCCUGGGCGCCA | |
| | UCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVFRSS | 46 |
| acid sequence | VLHSTQDLFLPFFSNVTWFHAIHVSGINGTKRFDNPVLPFNDGVYF | |
| | ASIEKSNIIRGWIFGTTLDSKTQSLLIVNNATNVVIKVCEFQFCND | |
| | PFLDVYYHKNNKSWMKSEFRVYSSANNCTFEYVSQPFLMDLEGKQG | |
| | NFKNLREFVFKNIDGYFKIYSKHTPINLVRDLPQGFSALEPLVDLP | |
| | IGINITRFQTLLALHRSYLTPGDSSSGWTAGAAAYYVGYLQPRTFL | |
| | LKYNENGTITDAVDCALDPLSETKCTLKSFTgggsgggQRVOPTES | |
| | IVRFPNITNLCPFGEVENATRFASVYAWNRKRISNCVADYSVLYNS | |
| | ASFSTFKCYGVSPTKLNDLCFTNVYADSFVIRGDEVRQIAPGQTGK | |
| | IADYNYKLPDDFTGCVIAWNSNNLDSKVGGNYNYRYRLFRKSNLKP | |
| | FERDISTEIYQAGSTPCNGVQGFNCYFPLQSYGFQPTNGVGYQPYR | |
| | VVVLSFELLHAPATVCGPKKSTNLVKNKCVNFNFNGLTGTGVLTES | |
| | NKKFLPFQQFGRDIADTTDAVRDPQTLEILDITPCSsgggsilaiy | |
| | stvasslvllvslgaisf | |
| PolyA tail | 100 nt | |

NTD-RBD-TM-Botswana_[A67V, A69-70, T95I, G142D/A143-145, A211/L212I, ins214EPE, G339D,
S371L, S373P, S375F, K417N, N440K, G446S, S477N, T478K, E484A, Q493K, G496S, Q498R,
N501Y, Y505H, T547K]

| SEQ ID NO: 55 consists of from 5′ end to 3′ end: 5′ UTR SEQ ID NO: 2, | | 55 |
|---|---|---|
| mRNA ORF SEQ ID NO: 56, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 56 |
| Construct | UGAACCUGACCACCCGGACCCAGCUGCCACCAGCCUACACCAACAG | |
| (excluding the stop | CUUCACCCGGGGCGUCUACUACCCCGACAAGGUGUUCCGGAGCAGC | |
| codon) | GUCCUGCACAGCACCCAGGACCUGUUCCUGCCCUUCUUCAGCAACG | |
| | UGACCUGGUUCCACGUGAUCAGCGGCACCAACGGCACCAAGCGGUU | |
| | CGACAACCCCGUGCUGCCCUUCAACGACGGCGUGUACUUCGCCAGC | |
| | AUCGAGAAGAGCAACAUCAUCCGGGGCUGGAUCUUCGGCACCACCC | |
| | UGGACAGCAAGACCCAGAGCCUGCUGAUCGUGAAUAACGCCACCAA | |
| | CGUGGUGAUCAAGGUGUGCGAGUUCCAGUUCUGCAACGACCCCUUC | |
| | CUGGACCACAAGAACAACAAGAGCUGGAUGGAGAGCGAGUUCCGGG | |
| | UGUACAGCAGCGCCAACAACUGCACCUUCGAGUACGUGAGCCAGCC | |
| | CUUCCUGAUGGACCUGGAGGGCAAGCAGGGCAACUUCAAGAACCUG | |
| | CGGGAGUUCGUGUUCAAGAACAUCGACGGCUACUUCAAGAUCUACA | |
| | GCAAGCACACCCCAAUCAUCGUGCGGGAGCCCGAGGAUCUGCCCCA | |
| | GGGCUUCUCAGCCCUGGAGCCCCUGGUGGACCUGCCCAUCGGCAUC | |
| | AACAUCACCCGGUUCCAGACCCUGCUGGCCCUGCACCGGAGCUACC | |
| | UGACCCCAGGCGACAGCAGCAGCGGGUGGACAGCAGGCGCGGCUGC | |
| | UUACUACGUGGGCUACCUGCAGCCCCGGACCUUCCUGCUGAAGUAC | |
| | AACGAGAACGGCACCAUCACCGACGCCGUGGACGGAGGCGGAUCGG | |
| | GAGGCGGACCCAACAUCACCAACCUGUGCCCCUUCGACGAGGUGUU | |
| | CAACGCCACCCGGUUCGCCAGCGUGUACGCCUGGAACCGGAAGCGG | |
| | AUCAGCAACUGCGUGGCCGACUACAGCGUGCUGUACAACCUGGCCC | |
| | CCUUCUUCACCUUCAAGUGCUACGGCGUGAGCCCCACCAAGCUGAA | |
| | CGACCUGUGCUUCACCAACGUGUACGCCGACAGCUUCGUGAUCCGU | |
| | GGCGACGAGGUGCGGCAGAUCGCACCCGGCCAGACAGGCAACAUCG | |
| | CCGACUACAACUACAAGCUGCCCGACGACUUCACCGGCUGCGUGAU | |
| | CGCCUGGAACAGCAACAAGCUCGACAGCAAGGUGAGCGGCAACUAC | |
| | AACUACCUGUACCGGCUGUUCCGGAAGAGCAACCUGAAGCCCUUCG | |
| | AGCGGGACAUCAGCACCGAGAUCUACCAAGCCGGCAACAAGCCUUG | |
| | CAACGGCGUGGCCGGCUUCAACUGCUACUUCCCUCUGCGGAGCUAC | |
| | AGCUUCCGGCCCACCUACGGCGUGGGCCACCAGCCCUACCGGGUGG | |
| | UGGUGCUGAGCUUCGAGCUGCUGCACGCCCCAGCCACCGUGUGUGG | |
| | CCCCAAGUCUGGCGGAGGCAGCAUCCUGGCCAUCUACAGCACCGUG | |
| | GCCAGCAGCCUGGUGCUGCUGGUGAGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVFRSS | 57 |
| acid sequence | VLHSTQDLFLPFFSNVTWFHVISGINGTKRFDNPVLPENDGVYFAS | |
| | IEKSNIIRGWIFGTTLDSKTQSLLIVNNATNVVIKVCEFQFCNDPF | |
| | LDHKNNKSWMESEFRVYSSANNCTFEYVSQPFLMDLEGKQGNFKNL | |
| | REFVFKNIDGYFKIYSKHTPIIVREPEDLPQGFSALEPLVDLPIGI | |
| | NITRFQTLLALHRSYLTPGDSSSGWTAGAAAYYVGYLQPRTFLLKY | |
| | NENGTITDAVDGGGSGGGPNITNLCPFDEVFNATRFASVYAWNRKR | |
| | ISNCVADYSVLYNLAPFFTFKCYGVSPTKLNDLCFTNVYADSFVIR | |
| | GDEVRQIAPGQTGNIADYNYKLPDDFTGCVIAWNSNKLDSKVSGNY | |
| | NYLYRLFRKSNLKPFERDISTEIYQAGNKPCNGVAGENCYFPLRSY | |
| | SFRPTYGVGHQPYRVVVLSFELLHAPATVCGPKSGGGSILAIYSTV | |
| | ASSLVLLVSLGAISF | |
| PolyA tail | 100 nt | |

NTD-RBD-TM-Botswana_v2[A67V, A69-70, T95I, G142D/A143-145, A211/L212I, ins214EPE,
G339D, S371L, S373P, S375F, K417N, N440K, G446S, S477N, T478K, E484A, Q493K, G496S,
Q498R, N501Y, Y505H, T547K]

| SEQ ID NO: 58 consists of from 5′ end to 3′ end: 5′ UTR SEQ ID NO: 2, | | 58 |
|---|---|---|
| mRNA ORF SEQ ID NO: 59, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 59 |
| Construct | UGAACCUGACCACCCGGACCCAGCUGCCUCCUGCCUACACCAACAG | |
| (excluding the stop | CUUCACCCGGGGCGUGUACUACCCCGACAAGGUGUUCCGGAGCAGC | |
| codon) | GUGCUGCACAGCACCCAGGACCUGUUCCUGCCCUUCUUCAGCAACG | |
| | UGACCUGGUUCCACGUGAUCAGCGGCACCAACGGCACCAAGCGGUU | |
| | CGACAACCCCGUGCUGCCCUUCAACGACGGCGUGUACUUCGCCAGC | |
| | AUCGAGAAGAGCAACAUCAUCCGGGGCUGGAUCUUCGGCACCACCC | |
| | UGGACAGCAAGACCCAGAGCCUGCUGAUCGUGAACAACGCCACCAA | |
| | CGUGGUGAUCAAGGUGUGCGAGUUCCAGUUCUGCAACGACCCCUUC | |
| | CUGGACCACAAGAACAACAAGAGCUGGAUGGAGAGCGAGUUCCGGG | |
| | UGUACAGCAGCGCCAACAACUGCACCUUCGAGUACGUGAGCCAGCC | |
| | CUUCCUGAUGGACCUGGAGGGCAAGCAGGGCAACUUCAAGAACCUG | |
| | CGGGAGUUCGUGUUCAAGAACAUCGACGGCUACUUCAAGAUCUACA | |
| | GCAAGCACACCCCUAUCAUCGUGCGGGAGCCCGAGGACCUGCCCCA | |
| | GGGCUUCAGCGCCCUGGAGCCCCUGGUGGACCUGCCCAUCGGCAUC | |
| | AACAUCACCCGGUUCCAGACCCUGCUGGCCCUGCACCGGAGCUACC | |
| | UGACCCCUGGCGACAGCAGCAGCGGCUGGACCGCCGGCGCCGCCGC | |
| | CUACUACGUGGGCUACCUGCAGCCCCGGACCUUCCUGCUGAAGUAC | |
| | AACGAGAACGGCACCAUCACCGACGCCGUGGACGGCGGCGGCAGCG | |
| | GCGGCGGCCCCAACAUCACCAACCUGUGCCCCUUCGACGAGGUGUU | |
| | CAACGCCACCCGGUUCGCCAGCGUGUACGCCUGGAACCGGAAGCGG | |
| | AUCAGCAACUGCGUGGCCGACUACAGCGUGCUGUACAACCUGGCCC | |
| | CCUUCUUCACCUUCAAGUGCUACGGCGUGAGCCCCACCAAGCUGAA | |
| | CGACCUGUGCUUCACCAACGUGUACGCCGACAGCUUCGUGAUCCGG | |
| | GGCGACGAGGUGCGGCAGAUCGCCCCCGGCCAGACCGGCAACAUCG | |
| | CCGACUACAACUACAAGCUGCCCGACGACUUCACCGGCUGCGUGAU | |
| | CGCCUGGAACAGCAACAAGCUGGACAGCAAGGUGAGCGGCAACUAC | |
| | AACUACCUGUACCGGCUGUUCCGGAAGAGCAACCUGAAGCCCUUCG | |
| | AGCGGGACAUCAGCACCGAGAUCUACCAGGCCGGCAACAAGCCCUG | |
| | CAACGGCGUGGCCGGCUUCAACUGCUACUUCCCUCUGCGGAGCUAC | |
| | AGCUUCCGGCCCACCUACGGCGUGGGCCACCAGCCCUACCGGGUGG | |
| | UGGUGCUGAGCUUCGAGCUGCUGCACGCCCCCGCCACCGUGUGCGG | |
| | CCCCAAGAGCGGCGGCGGCAGCAUCCUGGCCAUCUACAGCACCGUG | |
| | GCCAGCAGCCUGGUGCUGCUGGUGAGCCUGGGCGCCAUCAGCUUC | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVFRSS | 60 |
| acid sequence | VLHSTQDLFLPFFSNVTWFHVISGTNGTKRFDNPVLPENDGVYFAS | |
| | IEKSNIIRGWIFGTTLDSKTQSLLIVNNATNVVIKVCEFQFCNDPF | |
| | LDHKNNKSWMESEFRVYSSANNCTFEYVSQPFLMDLEGKQGNFKNL | |
| | REFVFKNIDGYFKIYSKHTPIIVREPEDLPQGFSALEPLVDLPIGI | |
| | NITRFQTLLALHRSYLTPGDSSSGWTAGAAAYYVGYLQPRTFLLKY | |
| | NENGTITDAVDGGGSGGGPNITNLCPFDEVENATRFASVYAWNRKR | |
| | ISNCVADYSVLYNLAPFFTFKCYGVSPTKLNDLCFTNVYADSFVIR | |
| | GDEVRQIAPGQTGNIADYNYKLPDDFTGCVIAWNSNKLDSKVSGNY | |
| | NYLYRLFRKSNLKPFERDISTEIYQAGNKPCNGVAGENCYFPLRSY | |
| | SFRPTYGVGHQPYRVVVLSFELLHAPATVCGPKSGGGSILAIYSTV | |
| | ASSLVLLVSLGAISF | |
| PolyA tail | 100 nt | |

NTD-RBD-TM-Botswana_v3[A67V, A69-70, T95I, G142D/A143-145, A211/L212I, ins214EPE,
G339D, S371L, S373P, S375F, K417N, N440K, G446S, S477N, T478K, E484A, Q493K, G496S,
Q498R, N501Y, Y505H, T547K]

| SEQ ID NO: 61 consists of from 5′ end to 3′ end: 5′ UTR SEQ ID NO: 2, | | 61 |
|---|---|---|
| mRNA ORF SEQ ID NO: 62, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 62 |
| Construct | UGAACCUGACCACCCGGACCCAGCUGCCACCAGCCUACACCAACAG | |
| (excluding the stop | CUUCACCCGGGGCGUGUACUACCCCGACAAGGUGUUCCGGAGCAGC | |
| codon) | GUGCUGCACAGCACCCAGGACCUGUUCCUGCCCUUCUUCAGCAACG | |
| | UGACCUGGUUCCACGUGAUCAGCGGCACCAACGGCACCAAGCGGUU | |
| | CGACAACCCCGUGCUGCCCUUCAACGACGGCGUGUACUUCGCCAGC | |
| | AUCGAGAAGAGCAACAUCAUCCGGGGCUGGAUCUUCGGCACCACCC | |
| | UGGACAGCAAGACCCAGAGCCUGCUGAUCGUGAACAACGCCACCAA | |
| | CGUGGUGAUCAAGGUGUGCGAGUUCCAGUUCUGCAACGACCCCUUC | |
| | CUGGACCACAAGAACAACAAGAGCUGGAUGGAGAGCGAGUUCCGGG | |
| | UGUACAGCAGCGCCAACAACUGCACCUUCGAGUACGUGAGCCAGCC | |
| | CUUCCUGAUGGACCUGGAGGGCAAGCAGGGCAACUUCAAGAACCUG | |
| | CGGGAGUUCGUGUUCAAGAACAUCGACGGCUACUUCAAGAUCUACA | |
| | GCAAGCACACCCCAAUCAUCGUGCGGGAGCCCGAGGACCUGCCCCA | |
| | GGGCUUCAGCGCCCUGGAGCCCCUGGUGGACCUGCCCAUCGGCAUC | |
| | AACAUCACCCGGUUCCAGACCCUGCUGGCCCUGCACCGGAGCUACC | |
| | UGACCCCAGGCGACAGCAGCAGCGGCUGGACCGCCGGCGCCGCCGC | |
| | CUACUACGUGGGCUACCUGCAGCCCCGGACCUUCCUGCUGAAGUAC | |
| | AACGAGAACGGCACCAUCACCGACGCCGUGGACGGCGGCGGCAGCG | |
| | GCGGCGGCCCCAACAUCACCAACCUGUGCCCCUUCGACGAGGUGUU | |
| | CAACGCCACCCGGUUCGCCAGCGUGUACGCCUGGAACCGGAAGCGG | |
| | AUCAGCAACUGCGUGGCCGACUACAGCGUGCUGUACAACCUGGCCC | |
| | CUUUCUUCACCUUCAAGUGCUACGGCGUGAGCCCCACCAAGCUGAA | |
| | CGACCUGUGCUUCACCAACGUGUACGCCGACAGCUUCGUGAUCCGG | |
| | GGCGACGAGGUGCGGCAGAUCGCCCCAGGCCAGACCGGCAACAUCG | |
| | CCGACUACAACUACAAGCUGCCCGACGACUUCACCGGCUGCGUGAU | |
| | CGCCUGGAACAGCAACAAGCUGGACAGCAAGGUGAGCGGCAACUAC | |
| | AACUACCUGUACCGGCUGUUCCGGAAGAGCAACCUGAAGCCCUUCG | |
| | AGCGGGACAUCAGCACCGAGAUCUACCAGGCCGGCAACAAGCCCUG | |
| | CAACGGCGUGGCCGGCUUCAACUGCUACUUCCCUCUGCGGAGCUAC | |
| | AGCUUCCGGCCCACCUACGGCGUGGGCCACCAGCCCUACCGGGUGG | |
| | UGGUGCUGAGCUUCGAGCUGCUGCACGCCCCAGCCACCGUGUGCGG | |
| | CCCCAAGAGCGGCGGCGGCAGCAUCCUGGCCAUCUACAGCACCGUG | |
| | GCCAGCAGCCUGGUGCUGCUGGUGAGCCUGGGCGCCAUCAGCUUU | |
| 3′ UTR | UGAUAAUAGGCUGGAGCCUCGGUGGCCUAGCUUCUUGCCCCUUGGG | 4 |
| | CCUCCCCCCAGCCCCUCCUCCCCUUCCUGCACCCGUACCCCCGUGG | |
| | UCUUUGAAUAAAGUCUGAGUGGGCGGC | |
| Corresponding amino | MFVFLVLLPLVSSQCVNLTTRTQLPPAYTNSFTRGVYYPDKVFRSS | 63 |
| acid sequence | VLHSTQDLFLPFFSNVTWFHVISGTNGTKRFDNPVLPENDGVYFAS | |
| | IEKSNIIRGWIFGTTLDSKTQSLLIVNNATNVVIKVCEFQFCNDPF | |
| | LDHKNNKSWMESEFRVYSSANNCTFEYVSQPFLMDLEGKQGNFKNL | |
| | REFVFKNIDGYFKIYSKHTPIIVREPEDLPQGFSALEPLVDLPIGI | |
| | NITRFQTLLALHRSYLTPGDSSSGWTAGAAAYYVGYLQPRTFLLKY | |
| | NENGTITDAVDGGGSGGGPNITNLCPFDEVFNATRFASVYAWNRKR | |
| | ISNCVADYSVLYNLAPFFTFKCYGVSPTKLNDLCFTNVYADSFVIR | |
| | GDEVRQIAPGQTGNIADYNYKLPDDFTGCVIAWNSNKLDSKVSGNY | |
| | NYLYRLFRKSNLKPFERDISTEIYQAGNKPCNGVAGENCYFPLRSY | |
| | SFRPTYGVGHQPYRVVVLSFELLHAPATVCGPKSGGGSILAIYSTV | |
| | ASSLVLLVSLGAISF | |
| PolyA tail | 100 nt | |

1283.B.1.1.529.IDR14C

| SEQ ID NO: 64 consists of from 5′ end to 3′ end: 5′ UTR SEQ ID NO: 2, | | 64 |
|---|---|---|
| mRNA ORF SEQ ID NO: 62, and 3′ UTR SEQ ID NO: 65. | | |
| Chemistry | 1 -methylpseudouridine | |
| Cap | 7mG(5′)ppp(5′)NlmpNp | |
| 5′ UTR | GGGAAAUAAGAGAGAAAAGAAGAGUAAGAAGAAAUAUAAGACCCCG | 2 |
| | GCGCCGCCACC | |
| ORF of mRNA | AUGUUCGUGUUCCUGGUGCUGCUGCCCCUGGUGAGCAGCCAGUGCG | 62 |
| Construct | UGAACCUGACCACCCGGACCCAGCUGCCACCAGCCUACACCAACAG | |
| (excluding the stop | CUUCACCCGGGGCGUGUACUACCCCGACAAGGUGUUCCGGAGCAGC | |
| codon) | GUGCUGCACAGCACCCAGGACCUGUUCCUGCCCUUCUUCAGCAACG | |
| | UGACCUGGUUCCACGUGAUCAGCGGCACCAACGGCACCAAGCGGUU | |
| | CGACAACCCCGUGCUGCCCUUCAACGACGGCGUGUACUUCGCCAGC | |
| | AUCGAGAAGAGCAACAUCAUCCGGGGCUGGAUCUUCGGCACCACCC | |
| | UGGACAGCAAGACCCAGAGCCUGCUGAUCGUGAACAACGCCACCAA | |
| | CGUGGUGAUCAAGGUGUGCGAGUUCCAGUUCUGCAACGACCCCUUC | |
| | CUGGACCACAAGAACAACAAGAGCUGGAUGGAGAGCGAGUUCCGGG | |
| | UGUACAGCAGCGCCAACAACUGCACCUUCGAGUACGUGAGCCAGCC | |
| | CUUCCUGAUGGACCUGGAGGGCAAGCAGGGCAACUUCAAGAACCUG | |
| | CGGGAGUUCGUGUUCAAGAACAUCGACGGCUACUUCAAGAUCUACA | |
| | GCAAGCACACCCCAAUCAUCGUGCGGGAGCCCGAGGACCUGCCCCA | |
| | GGGCUUCAGCGCCCUGGAGCCCCUGGUGGACCUGCCCAUCGGCAUC | |
| | AACAUCACCCGGUUCCAGACCCUGCUGGCCCUGCACCGGAGCUACC | |
| | UGACCCCAGGCGACAGCAGCAGCGGCUGGACCGCCGGCGCCGCCGC | |
| | CUACUACGUGGGCUACCUGCAGCCCCGGACCUUCCUGCUGAAGUAC | |
| | AACGAGAACGGCACCAUCACCGACGCCGUGGACGGCGGCGGCAGCG | |
| | GCGGCGGCCCCAACAUCACCAACCUGUGCCCCUUCGACGAGGUGUU | |
| | CAACGCCACCCGGUUCGCCAGCGUGUACGCCUGGAACCGGAAGCGG | |
| | AUCAGCAACUGCGUGGCCGACUACAGCGUGCUGUACAACCUGGCCC | |
| | CUUUCUUCACCUUCAAGUGCUACGGCGUGAGCCCCACCAAGCUGAA | |
| | CGACCUGUGCUUCACCAACGUGUACGCCGACAGCUUCGUGAUCCGG | |
| | GGCGACGAGGUGCGGCAGAUCGCCCCAGGCCAGACCGGCAACAUCG | |
| | CCGACUACAACUACAAGCUGCCCGACGACUUCACCGGCUGCGUGAU | |
| | CGCCUGGAACAGCAACAAGCUGGACAGCAAGGUGAGCGGCAACUAC | |
| | AACUACCUGUACCGGCUGUUCCGGAAGAGCAACCUGAAGCCCUUCG | |
| | AGCGGGACAUCAGCACCGAGAUCUACCAGGCCGGCAACAAGCCCUG | |
| | CAACGGCGUGGCCGGCUUCAACUGCUACUUCCCUCUGCGGAGCUAC | |
| | AGCUUCCGGCCCACCUACGGCGUGGGCCACCAGCCCUACCGGGUGG | |
| | UGGUGCUGAGCUUCGAGCUGCUGCACGCCCCAGCCACCGUGUGCGG | |
| | CCCCAAGAGCGGCGGCGGCAGCAUCCUGGCCAUCUACAGCACCGUG | |
| | GCCAGCAGCCUGGUGCUGCUGGUGAGCCUGGGCGCCAUCAGCUUU | |
| 3′ UTR | UGAUAAUAGGCUGGAGCCUCGGUGGCCUAGCUUCUUGCCCCUUGGG | 65 |
| | CCUCCCCCCAGCCCCUCCUCCCCUUCCUGCAGGAGUGAGAGAGAAG | |
| | UGGUCUUUGAAUAAAGUCUGAGUGGGCGGC | |
| Corresponding amino | MFVFLVLLPLVSSQCVNLTTRTQLPPAYTNSFTRGVYYPDKVFRSS | 63 |
| acid sequence | VLHSTQDLFLPFFSNVTWFHVISGINGTKRFDNPVLPENDGVYFAS | |
| | IEKSNIIRGWIFGTTLDSKTQSLLIVNNATNVVIKVCEFQFCNDPF | |
| | LDHKNNKSWMESEFRVYSSANNCTFEYVSQPFLMDLEGKQGNFKNL | |
| | REFVFKNIDGYFKIYSKHTPIIVREPEDLPQGFSALEPLVDLPIGI | |
| | NITRFQTLLALHRSYLTPGDSSSGWTAGAAAYYVGYLQPRTFLLKY | |
| | NENGTITDAVDGGGSGGGPNITNLCPFDEVFNATRFASVYAWNRKR | |
| | ISNCVADYSVLYNLAPFFTFKCYGVSPTKLNDLCFTNVYADSFVIR | |
| | GDEVRQIAPGQTGNIADYNYKLPDDFTGCVIAWNSNKLDSKVSGNY | |
| | NYLYRLFRKSNLKPFERDISTEIYQAGNKPCNGVAGENCYFPLRSY | |
| | SFRPTYGVGHQPYRVVVLSFELLHAPATVCGPKSGGGSILAIYSTV | |
| | ASSLVLLVSLGAISF | |
| PolyA tail | 100 nt | |

### EQUIVALENTS

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

What is claimed is:

1. A nucleic acid encoding a SARS-CoV-2 antigen of a second circulating SARS-CoV-2 virus strain, wherein the antigen has an amino acid sequence with at least one amino acid mutation with respect to an antigen of a first circulating SARS-CoV-2 virus strain, wherein the mutation is an amino acid substitution, deletion or insertion, wherein the antigen is not a full length stabilized spike protein having a 2P mutation and wherein the nucleic acid is in a lipid nanoparticle.

2. The nucleic acid of claim 1, wherein the first and second virus strains are in circulation for at least a portion of 1 year.

3. The nucleic acid of claim 1, wherein the first and second virus strains are in circulation during the same pandemic period or endemic period.

4. The nucleic acid of any one of claims 1-3, wherein the SARS-CoV-2 antigen has a second amino acid mutation with respect to the antigen of the first circulating SARS-CoV-2 virus strain, wherein the mutation is an amino acid substitution, deletion or insertion, and the second mutation corresponds to a third virus strain in circulation.

5. The nucleic acid of any one of claims 1-4, wherein the lipid nanoparticle comprises an ionizable amino lipid, a sterol, a neutral lipid, and a polyethylene glycol (PEG)-modified lipid.

6. The nucleic acid of claim 5, wherein the lipid nanoparticle comprises 40-55 mol % ionizable amino lipid, 30-45 mol % sterol, 5-15 mol % neutral lipid, and 1-5 mol % PEG-modified lipid.

7. The nucleic acid of any one of claims 1-6, wherein the SARS-CoV-2 antigen is a receptor binding domain (RBD) of spike protein.

8. The nucleic acid of any one of claims 1-6, wherein the SARS-CoV-2 antigen is an N-terminal domain (NTD).

9. The nucleic acid of any one of claims 1-6, wherein the SARS-CoV-2 antigen is a combination of an RBD and NTD.

10. The nucleic acid of any one of claims 1-6, wherein the SARS-CoV-2 antigen is an NTD, RBD, and transmembrane domain joined by linkers.

11. The nucleic acid of claim 10, wherein the SARS-CoV-2 antigen is an NTD, RBD, and influenza hemagglutinin transmembrane (HATM) domain joined by linkers (NTD-RBD-HATM).

12. The nucleic acid of any one of claims 1-6, wherein the SARS-CoV-2 antigen is an NTD-RBD fusion.

13. The nucleic acid of any one of claims 1-6, wherein the SARS-CoV-2 antigen is an S1 protein subunit.

14. The nucleic acid of any one of claims 1-13, wherein the amino acid mutation in the antigen is associated with a structure or function of the virus that is different in quantity or kind from a corresponding structure or function in the first virus.

15. The nucleic acid of claim 14, wherein the function of the amino acid mutation is ACE2 receptor binding, virus transmissibility, viral uptake, viral pathogenesis, altered furin cleavage, or viral replication.

16. The nucleic acid of any one of claims 1-15, wherein the nucleic acid comprises messenger RNA (mRNA).

17. A composition comprising:
a first messenger ribonucleic acid (mRNA) encoding a first SARS-CoV-2 antigen of a first SARS-CoV-2 virus and a second mRNA encoding a second SARS-CoV-2 antigen of a second SARS-CoV-2 virus, wherein the second SARS-CoV-2 virus has an amino acid sequence with at least one amino acid mutation with respect to an amino acid sequence of the first SARS-CoV-2 virus, wherein the mutation is an amino acid substitution, deletion or insertion, wherein each of the first and second antigens are not full length stabilized spike proteins having a 2P mutation.

18. The composition of claim 17, wherein the first SARS-CoV-2 virus is a first circulating SARS-CoV-2 virus strain, and the second SARS-CoV-2 virus is a second circulating SARS-CoV-2 virus strain, and wherein the first and second virus strains are spreading in the population for at least a portion of 1 year.

19. The composition of any one of claims 17-18, wherein the mRNAs are present at about a 1:1 ratio relative to each other mRNA in the composition.

20. The composition of any one of claims 17-19, wherein the mRNAs are in a lipid nanoparticle and wherein the lipid nanoparticle comprises an ionizable amino lipid, a sterol, a neutral lipid, and a polyethylene glycol (PEG)-modified lipid.

21. The composition of claim 20, wherein the lipid nanoparticle comprises 40-55 mol % ionizable amino lipid, 30-45 mol % sterol, 5-15 mol % neutral lipid, and 1-5 mol % PEG-modified lipid.

22. The composition of any one of claims 20-21, wherein the lipid nanoparticle comprises 40-50 mol % ionizable amino lipid, 35-45 mol % sterol, 10-15 mol % neutral lipid, and 2-4 mol % PEG-modified lipid.

23. The composition of any one of claims 20-22, wherein the lipid nanoparticle comprises 45 mol %, 46 mol %, 47 mol %, 48 mol %, 49 mol %, or 50 mol % ionizable amino lipid.

24. The composition of any one of claims 20-23, wherein the ionizable amino lipid has the structure of Compound 1:

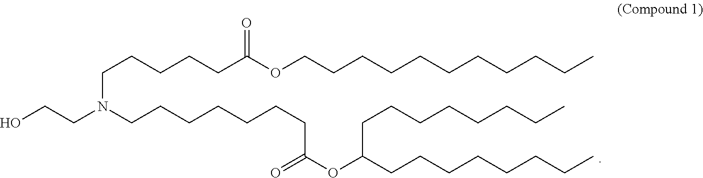 [SMILES: CCCCCCCCCCCOC(=O)CCCCCN(CCO)CCCCCCCC(=O)OC(CCCCCCCC)CCCCCCCC]

25. The composition of any one of claims 20-24, wherein the sterol is cholesterol or a variant thereof.

26. The composition of any one of claims 20-25, wherein the neutral lipid is 1,2 distearoyl-sn-glycero-3-phosphocholine (DSPC).

27. The composition of any one of claims 20-26, wherein the PEG-modified lipid is 1,2 dimyristoyl-sn-glycerol, methoxypolyethyleneglycol (PEG2000 DMG).

28. A method comprising
administering to a subject a second effective dose of a second vaccine comprising a second nucleic acid encoding a second SARS-CoV-2 antigen, wherein the subject has previously been administered a first effective dose of a first vaccine comprising a first nucleic acid encoding a first SARS-CoV-2 antigen,
wherein the second vaccine is administered in an effective amount to induce an immune response specific for the second antigen, wherein the second SARS-CoV-2 antigen has an amino acid sequence with at least one mutation, with respect to a corresponding amino acid sequence of the first SARS-CoV-2 antigen, wherein the mutation is an amino acid substitution, deletion or insertion and wherein each of the first and second antigens are not full length stabilized spike proteins having a 2P mutation.

29. The method of claim 28, wherein the first encoded SARS-CoV-2 antigen is of a first circulating SARS-CoV-2 virus and wherein the second encoded SARS-CoV-2 antigen is of a second circulating SARS-CoV-2 virus.

30. The method of claim 28, wherein the first encoded SARS-CoV-2 antigen is representative of a first circulating SARS-CoV-2 virus and wherein the second encoded SARS-CoV-2 antigen is representative of a second circulating SARS-CoV-2 virus.

31. The method of claim 28, wherein the first encoded SARS-CoV-2 antigen is representative of a plurality of first circulating SARS-CoV-2 viruses and/or wherein the second encoded SARS-CoV-2 antigen is representative of a second plurality of circulating SARS-CoV-2 viruses.

32. The method of any one of claims 29-32, wherein the second circulating SARS-CoV-2 virus is an immunodominant emerging strain or variant of concern detected during a period when the first circulating SARS-CoV-2 virus is present in a subject population.

33. The method of any one of claims 29-32, wherein the second circulating SARS-CoV-2 virus and the first circulating SARS-CoV-2 virus are detectable in a subject population within at least one year.

34. The method of any one of claims 29-32, wherein the second circulating SARS-CoV-2 virus and the first circulating SARS-CoV-2 virus are detectable in a subject population during a same season.

35. The method of any one of claims 29-32, wherein the second circulating SARS-CoV-2 virus and the first circulating SARS-CoV-2 virus are detectable in a subject population during a same pandemic period or endemic period.

36. The method of any one of claims 28-35, wherein the first nucleic acid encoding the first SARS-CoV-2 antigen is a DNA or a messenger RNA (mRNA).

37. The method of any one of claims 28-36, wherein the first nucleic acid encoding a first SARS-CoV-2 antigen is a messenger RNA (mRNA).

38. The method of any one of claims 28-36 wherein the second nucleic acid encoding a second SARS-CoV-2 antigen is a messenger RNA (mRNA).

39. The method of any one of claims 28-38, wherein the second vaccine further comprises the first nucleic acid encoding a first SARS-CoV-2 antigen.

40. The method of claim 39, wherein the first nucleic acid encoding a first SARS-CoV-2 antigen and the second nucleic acid encoding a second SARS-CoV-2 antigen are present in the second vaccine in a 1:1 ratio.

41. The method of any one of claims 28-38, wherein the first and/or second vaccine is a monovalent vaccine.

42. The method of any one of claims 28-40, wherein the first and/or second vaccine is a multivalent vaccine, comprising more than one nucleic acids encoding antigens representative of a plurality of variants of concern.

43. The method of any one of claims 28-42, wherein the first encoded SARS-CoV-2 antigen is administered to the subject as a first vaccine comprised of one or more doses and the second encoded SARS-CoV-2 antigen is administered to the subject as a boost in one or more doses.

44. The method of any one of claims 28-42, wherein the second encoded SARS-CoV-2 antigen is administered to the subject as first vaccine comprised of one or more doses and the first encoded SARS-CoV-2 antigen is administered to the subject as a boost in one or more doses.

45. The method of any one of claims 28-42, wherein the first and second encoded SARS-CoV-2 antigens are administered to the subject together as a boost dose, optionally wherein the first and second encoded SARS-CoV-2 antigens are present in the boost dose in a 1:1 ratio.

46. The method of any one of claims 28-42, wherein the first encoded SARS-CoV-2 antigen is administered to the subject as a prime dose and as a boost dose to complete a vaccination and wherein the second encoded SARS-CoV-2 antigen is administered to the subject as a booster regimen at least 3 months after the first vaccination is complete.

47. The method of any one of claims 28-42, wherein the first encoded SARS-CoV-2 antigen is administered to the subject as a prime dose and as a boost dose in an initial vaccination and the second encoded SARS-CoV-2 antigen is administered to the subject as a boost dose more than 6 months after the initial vaccination.

48. The method of any one of claims 46-47, wherein the boost dose is a seasonal boost or a pandemic shift boost to provide protection for a plurality of variants of concern.

49. A method comprising
administering to a subject a vaccine comprising a first nucleic acid encoding a SARS-CoV-2 antigen of a first SARS-CoV-2 virus, wherein the first SARS-CoV-2 virus is an emerging variant of a primary SARS-CoV-2 virus, wherein the antigen has an amino acid sequence with at least one amino acid mutation with respect to a corresponding protein antigen of the primary SARS-CoV-2 virus, and wherein the mutation is an amino acid substitution, deletion or insertion, and wherein the subject is seropositive for a SARS-CoV-2 antigen of the primary SARS-CoV-2 virus.

50. A method comprising
administering to a subject a vaccine comprising a first nucleic acid encoding a SARS-CoV-2 antigen of a first SARS-CoV-2 virus, wherein the first SARS-CoV-2 virus is an emerging variant of a primary SARS-CoV-2 virus, wherein the antigen has an amino acid sequence with at least one amino acid mutation with respect to a corresponding protein antigen of the primary SARS-CoV-2 virus, and wherein the mutation is an amino acid substitution, deletion or insertion, and wherein the subject is seronegative for a SARS-CoV-2 antigen of the primary SARS-CoV-2 virus.

51. The method of claim 49 or 50, wherein the subject is administered a second dose of the vaccine between 2 weeks and 1 year after the first dose of vaccine is administered.

52. The method of claim 49 or 50, wherein the subject is administered a second vaccine between 2 weeks and 1 year after the vaccine is administered, wherein the second vaccine comprises a second nucleic acid encoding the corresponding protein antigen of the primary SARS-CoV-2 virus.

53. The method of claim 52, wherein the second vaccine comprises a mixture of the first and second nucleic acids, wherein the first nucleic acid and the second nucleic acid are present in the second vaccine at a ratio of 1:1.

54. A method comprising
administering to a subject a booster vaccine comprising a nucleic acid encoding a first SARS-CoV-2 antigen from a first SARS-CoV-2 virus, wherein the subject has previously been administered at least one prime dose of a first vaccine comprising a first nucleic acid encoding the SARS-CoV-2 antigen of the first the SARS-CoV-2 virus, wherein the booster vaccine is administered in an effective amount to induce a neutralizing immune response against a second SARS-CoV-2 virus, wherein the second SARS-CoV-2 virus comprises a second SARS-CoV-2 antigen, wherein the second BARS-CoV-2 antigen has an amino acid sequence with at least one amino acid mutation with respect to a corresponding protein antigen of the first BARS-CoV-2 virus, wherein the booster vaccine is administered in a dosage of 25-100 μg at least 6 months after a first dose of the first vaccine, and wherein each of the first and second antigens are not full length stabilized spike proteins having a 0.2P mutation.

55. The method of claim 54, wherein the booster vaccine is administered in a dosage of 50 μg.

56. The method of claim 54 or 55, wherein the booster vaccine is administered at least about 6 months after a second dose of the first vaccine.

57. The method of claim 54 or 55, wherein the booster vaccine is administered 6-12 months after a second dose of the first vaccine.

58. The method of claim 54 or 55, wherein the booster vaccine is administered at least about 8 months after a second dose of the first vaccine.

59. The method of any one of claims 54-58, wherein the boost dose is a seasonal boost or a pandemic shift boost to provide a neutralizing immune response against a plurality of variants of concern.
